(19) United States
(12) Patent Application Publication
(10) Pub. No.: US 2018/0207164 A1
(43) Pub. Date: Jul. 26, 2018

(54) COMBINATION THERAPIES FOR TREATING CANCERS

(71) Applicant: GILEAD SCIENCES, INC., Foster City, CA (US)

(72) Inventors: Esteban M. Abella, Foster City, CA (US); Helen Collins, Atherton, CA (US); Julie DiPaolo, Foster City, CA (US); Christophe Quéva, Braine L'Alleud 1420 (BE); Daniel Tumas, Foster City, CA (US)

(21) Appl. No.: 15/749,222

(22) PCT Filed: Jul. 22, 2016
(86) PCT No.: PCT/US2016/043676

§ 371 Date: Jan. 31, 2018

Related U.S. Application Data
(60) Provisional application No. 62/200,549, filed on Aug. 3, 2015.
(60) Provisional application No. 62/200,545, filed on Aug. 3, 2015.
(60) Provisional application No. 62/200,556, filed on Aug. 3, 2015.
(60) Provisional application No. 62/201,009, filed on Aug. 4, 2015.
(60) Provisional application No. 62/235,102, filed on Sep. 30, 2015.

(51) Int. Cl. A61K 31/522; A61P 35/00; A61K 45/06; A61K 31/52; A61K 39/395; C07K 16/28; A61K 31/635

(52) U.S. Cl. CPC A61K 31/522; A61P 35/00; A61K 45/06; A61K 2039/505; A61K 39/39541; C07K 16/2887; A61K 31/635; A61K 31/52

(57) ABSTRACT

Methods for treatment of cancer, including hematological malignancies. In particular, the methods include administration of a BTK inhibitor and a BCL-2 inhibitor or pharmaceutically acceptable salts or hydrates thereof, a Btk inhibitor and a Syk inhibitor or pharmaceutically acceptable salts or hydrates thereof, and administration of a Btk inhibitor and a P13K inhibitor or pharmaceutically acceptable salts or hydrates thereof.

### FIELD

The present disclosure relates generally to therapeutics and compositions for treating cancers, and more specifically to the use of Bruton's Tyrosine Kinase (BTK) inhibitors in combination with B-cell chronic lymphocytic leukemia (CLL)/lymphoma 2 (BCL-2) inhibitors for treating cancers. The present disclosure also relates to the use of a phosphatidylinositol 3-kinase (PI3K) inhibitor in combination with B-cell chronic lymphocytic leukemia (CLL)/lymphoma 2 (BCL-2) inhibitors for treating cancers.

### BACKGROUND

BTK inhibitors useful in treating hematological cancers include those taught in U.S. Pat. No. 8,940,725 (Yamamoto et al.) and U.S. Pat. No. 7,514,444 (Honigberg et al.). Entospletinib, also known as GS-9973, is a Syk inhibitor in clinical development, the synthesis of which can be seen in U.S. Pat. Nos. 8,450,321 and 8,455,493. WO 2014/168975 teaches a combination of the Btk inhibitor ibrutinib with a Syk inhibitor, specifically referencing R406, and with a Bcl-2 inhibitor, including ABT-737, ABT-199, and HA14-1

Various compounds that inhibit the activity of anti-apoptotic BCL proteins are known in the art. Several BCL-2-selective apoptosis inducing compounds may be used in treating cancer. However, some BCL-2 inhibitors may cause thrombocytopenia and have limited use in clinical treatments (see e.g., Zhang et al., Cell Death and Differentiation 14: 943-951, 2007). Thus, there remains a need for alternative therapies to treat cancer in humans.

There remains a need for alternative therapies to treat cancer in humans

### BRIEF SUMMARY

Provided herein are methods for treating cancer that involve the administration of a BTK inhibitor in combination with a BCL-2 inhibitor. In some aspects, provided is a method for treating cancer in a human in need thereof, comprising administering to the human a therapeutically effective amount of a BTK inhibitor and a therapeutically effective amount of a BCL-2 inhibitor.

Provided herein are methods for treating cancer that involve the administration of a BTK inhibitor in combination with a Syk inhibitor. In some aspects, provided is a method for treating cancer in a human in need thereof, comprising administering to the human a therapeutically effective amount of a BTK inhibitor and a therapeutically effective amount of a Syk inhibitor.

In some embodiments, the BTK inhibitor is 6-amino-9-[(3R)-1-(2-butynoyl)-3-pyrrolidinyl]-7-(4-phenoxyphenyl)-7,9-dihydro-8H-purin-8-one, or a pharmaceutically acceptable salt or hydrate thereof.

In some variations, the BTK inhibitor is a hydrochloride salt of 6-amino-9-[(3R)-1-(2-butynoyl)-3-pyrrolidinyl]-7-(4-phenoxyphenyl)-7,9-dihydro-8H-purin-8-one, or a pharmaceutically acceptable hydrate thereof.

In some embodiments, the BCL-2 inhibitor is selected from the group of:

(4-(4-{[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfnyl)-2-(1H-pyryrrolo[2,3-b]pyridin-5-yl-oxy)benzamide), also known as ABT-199, GDC 0199, and Venetoclax, and is referred to herein as Compound B1;

4-(4-((4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide, referred to herein as Compound B2; and

4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl) sulfonyl)benzamide, referred to herein as Compound B3,

or a pharmaceutically acceptable salt or hydrate thereof.

In some embodiments, the Syk inhibitor is 6-(1H-indazol-6-yl)-N-(4-morpholinophenyl)imidazo[1,2-a]pyrazin-8-amine, or a pharmaceutically acceptable salt or hydrate thereof. In some variations, the Syk inhibitor is a mesylate salt of 6-(1H-indazol-6-yl)-N-(4-morpholinophenyl)imidazo[1,2-a]pyrazin-8-amine, or a hydrate thereof or a pharmaceutically acceptable salt thereof.

Provided herein are also articles of manufacture and kits that comprise the BTK inhibitor and the Syk inhibitors described herein.

Provided herein are also articles of manufacture and kits that comprise the BTK inhibitor and the BCL-2 inhibitors described herein.

Provided herein are methods for treating B-cell malignancies that involve the administration of a therapeutically effective amount of 2-(1-((9H-purin-6-yl)amino)propyl)-5-fluoro-3-phenylquinazolin-4(3H)-one, or a pharmaceutically acceptable salt thereof, and a therapeutically effective amount of (4-(4-{[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide), or a pharmaceutically acceptable salt thereof.

2-(1-((9H-Purin-6-yl)amino)propyl)-5-fluoro-3-phenylquinazolin-4(3H)-one, or a pharmaceutically acceptable salt thereof, is an example of a PI3K inhibitor. In certain variations, the 2-(1-((9H-purin-6-yl)amino)propyl)-5-fluoro-3-phenylquinazolin-4(3H)-one, or a pharmaceutically acceptable salt thereof is administered to the human at a dose between 50 mg and 150 mg.

(4-(4-{[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide), or a pharmaceutically acceptable salt thereof, is an example of a BCL-2 inhibitor. It is also known as ABT-199, GDC 0199, and Venetoclax, and is referred to herein as Compound B1. In certain variations, the (4-(4-{[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide), or a pharmaceutically acceptable salt thereof, is administered to the human at a dose between 20 mg and 400 mg. In certain variations, the (4-(4-{[2-(4-chlorophenyl)-4,4-dimethylcyclohex-1l-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide), or a pharmaceutically acceptable salt thereof, is administered to the human at a dose between 50 mg and 150 mg once or twice daily. In certain variations, Compound B1 is administered to the human at a dose between 20 and 600 mg at a regimen that includes daily or weekly dosing or combinations thereof. In certain variations, Compound B1 is administered to the human at a regimen that includes a daily lead-in dose that changes to a weekly dose after a specified period of time or dosages.

Provided herein are also pharmaceutical compositions, articles of manufacture and kits that comprise the PI3K inhibitor and the BCL-2 inhibitor described herein.

Provided herein are methods for treating cancer that involve the administration of a BTK inhibitor in combination with a PI3K delta inhibitor. In some embodiments the combination also comprises obinutuzumab. Provided herein are methods for treating cancer that involve the administration of BTK inhibitor in combination with a PI3K delta inhibitor. In some embodiments the combination also comprises ABT-199. Provided herein are methods for treating cancer that involve the administration of BTK inhibitor in combination with Syk inhibitor. In some embodiments the combination also comprises obinutuzumab. Provided herein are methods for treating cancer that involve the administration of BTK inhibitor in combination with Syk inhibitor. In some embodiments the combination also comprises ABT-199. Provided herein are methods for treating cancer that involve the administration of BTK inhibitor in combination with ABT-199 and obinutuzumab. Also provided are methods for treating cancer that involve the administration of PI3K delta inhibitor in combination with ABT-199 and obinutuzumab. Furthermore, provided are methods for treating cancer that involve the administration of Syk inhibitor in combination with ABT-199 and obinutuzumab.

### BRIEF DESCRIPTION OF THE FIGURES

FIG. 1 shows that HS-5 stromal cell co-culture protects CLL cells from apoptosis in culture.

FIG. 2 shows that αIgM/αIgG/αCD40 stimulation protected CLL cells from apoptosis.

FIGS. 3 and 4 show that Compound A1 induced apoptosis in primary CLL cells from donors stimulated with αIgM/αIgG/αCD40.

FIG. 5 shows the apoptosis percentage in CLL cells treated with Compounds A1 or Compound B1.

FIG. 6 shows that the apoptosis percentage in CLL cells treated with Compound A1 and Compound B1.

FIG. 7 shows theapoptosis percentage in CLL cells treated with Compounds C1 or Compound B1.

FIG. 8 shows the apoptosis percentage in CLL cells treated with Compound C1 and Compound B1.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Provided herein is a method for treating cancer in a human in need thereof, comprising administering to the human a therapeutically effective amount of a BTK inhibitor and a therapeutically effective amount of a BCL-2 inhibitor. Provided are also compositions (including pharmaceutical compositions, formulations, or unit dosages), articles of manufacture and kits comprising a BTK inhibitor and a BCL-2 inhibitor.

Provided herein is a method for treating cancer in a human in need thereof, comprising administering to the human a therapeutically effective amount of a Btk inhibitor and a therapeutically effective amount of a Syk inhibitor. Provided are also compositions (including pharmaceutical compositions, formulations, or unit dosages), articles of manufacture and kits comprising a Btk inhibitor and a Syk inhibitor.

Provided herein is a method for treating cancer in a human in need thereof, comprising administering to the human a therapeutically effective amount of a Syk inhibitor and a therapeutically effective amount of a BCL-2 inhibitor. Provided are also compositions (including pharmaceutical compositions, formulations, or unit dosages), articles of manufacture and kits comprising a Syk inhibitor and a BCL-2 inhibitor.

Provided herein is a method for treating cancer in a human in need thereof, comprising administering to the human a therapeutically effective amount of a PI3K-δ inhibitor and a therapeutically effective amount of a BCL-2 inhibitor. Provided are also compositions (including pharmaceutical compositions, formulations, or unit dosages), articles of manufacture and kits comprising a PI3K-δ inhibitor and a BCL-2 inhibitor.

Also provided herein is a method for treating cancer in a human in need thereof comprising administering to the human a Btk inhibitor and a PI3K-δ inhibitor. Provided are also compositions (including pharmaceutical compositions, formulations, or unit dosages), articles of manufacture and kits comprising a Btk inhibitor and a PI3K-δ inhibitor.

Further provided herein is a method for treating cancer in a human in need thereof comprising administering to the human a Btk inhibitor, a PI3K-δ inhibitor, and a CD20 inhibitor. Provided are also compositions (including pharmaceutical compositions, formulations, or unit dosages), articles of manufacture and kits comprising a Btk inhibitor, a PI3K-δ inhibitor, and a CD20 inhibitor. Further provided herein is a method for treating cancer in a human in need thereof comprising administering to the human a Btk inhibitor, a Syk inhibitor, and a CD20 inhibitor. Provided are also compositions (including pharmaceutical compositions, formulations, or unit dosages), articles of manufacture and kits comprising a Btk inhibitor, a Syk inhibitor, and a CD20 inhibitor. Further provided herein is a method for treating cancer in a human in need thereof comprising administering to the human a PI3K-δ inhibitor, a BCL-2 inhibitor, and a CD20 inhibitor. Provided are also compositions (including pharmaceutical compositions, formulations, or unit dosages), articles of manufacture and kits comprising PI3K-δ inhibitor, a BCL-2 inhibitor, an anti-CD20 antibody. Further provided herein is a method for treating cancer in a human in need thereof comprising administering to the human a Syk inhibitor, a BCL-2 inhibitor, and a CD20 inhibitor. Provided are also compositions (including pharmaceutical compositions, formulations, or unit dosages), articles of manufacture and kits comprising a Syk inhibitor, a BCL-2 inhibitor, and a CD20 inhibitor. Further provided herein is a method for treating cancer in a human in need thereof comprising administering to the human a Btk inhibitor, a BCL-2 inhibitor, and a CD20 inhibitor. Provided are also compositions (including pharmaceutical compositions, formulations, or unit dosages), articles of manufacture and kits comprising a Btk inhibitor, a BCL-2 inhibitor, and a CD20 inhibitor.

### Compounds

In some variations, the BTK inhibitor is Compound A1, or a pharmaceutically acceptable salt or hydrate thereof. Compound A1 has the structure:

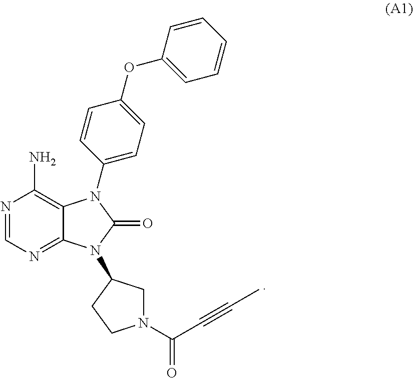 [SMILES: CC#CC(=O)N1CC[C@@H](n2c(=O)n(-c3ccc(Oc4ccccc4)cc3)c3c(N)ncnc32)C1]

In some variations, the BTK inhibitor is a hydrochloride salt of Compound A1, or a hydrate thereof. Compound A1 may be synthesized according to the methods described in U.S. Pat. No. 8,557,803 (Yamamoto et al.) and US 2014/0330015. Compound A1 may be referred to as (R)-6-amino-9-(1-(but-2-ynoyl)pyrrolidin-3-yl)-7-(4-phenoxyphenyl)-7H-purin-8(9H)-one or 6-amino-9-[(3R)-1-(2-butynoyl)-3-pyrrolidinyl]-7-(4-phenoxyphenyl)-7,9-dihydro-8H-purin-8-one.

Additional Btk inhibitors may include, but are not limited to, ibrutinib, acalabrutinib, HM71224, CNX-774, RN486, and CC-292.

2-(1-((9H-Purin-6-yl)amino)propyl)-5-fluoro-3-phenylquinazolin-4(3H)-one, or a pharmaceutically acceptable salt thereof, is an example of a PI3K inhibitor, and more specifically, a PI3 kinase delta-specific isoform (PI3Kδ) inhibitor. Such compound is also referred to in the art as idelalisib, and referred to herein as Compound C1, and has the structure:

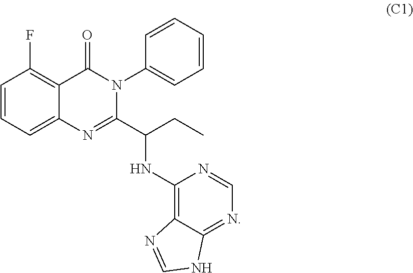 [SMILES: CCC(Nc1ncnc2[nH]cnc12)c1nc2cccc(F)c2c(=O)n1-c1ccccc1]

In one variation, Compound C1 is predominantly the S-enantiomer, having the structure:

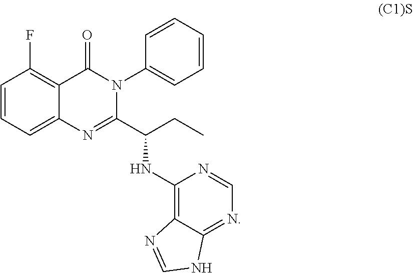 [SMILES: CC[C@H](Nc1ncnc2[nH]cnc12)c1nc2cccc(F)c2c(=O)n1-c1ccccc1]

The (S)-enantiomer of Compound C1 may also be referred to by its compound name: (S)-2-(1-((9H-purin-6-yl)amino)propyl)-5-fluoro-3-phenylquinazolin-4(3H)-one. Compound C1 may be synthesized according to the methods described in U.S. Pat. No. 7,932,260.

Additional PI3K (phosphoinositide 3-kinase) inhibitors may include, but are not limited to, inhibitors of PI3Kγ, PI3Kδ, PI3Kβ, PI3Kα, and/or pan-PI3K. Examples of PI3K inhibitors include, but are not limited to, wortmannin, BKM120, CH5132799, XL756, and GDC-0980. Examples of PI3Kγ inhibitors include, but are not limited to, ZSTK474, AS252424, LY294002, and TG100115. Examples of PI3K6 inhibitors include, but are not limited to, PI3K II, TGR-1202, AMG-319, GSK2269557, X-339, X-414, RP5090, KAR4141, XL499, OXY111A, duvelisib (or IPI-145), IPI-443, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences). Examples of PI3Kβ inhibitors include, but are not limited to, GSK2636771, BAY 10824391, and TGX221. Examples of PI3Kα inhibitors include, but are not limited to, buparlisib, BAY 80-6946, BYL719, PX-866, RG7604, MLN1117, WX-037, AEZA-129, and PA799. Examples of pan-PI3K inhibitors include, but are not limited to, LY294002, BEZ235, XL147 (SAR245408), and GDC-0941.

In some variations, the BCL-2 inhibitor is Compound B1, Compound B2, or Compound B3, or a pharmaceutically acceptable salt thereof.

Compound B1 which is also known as ABT-199, 4-[4-[[2-(4-chlorophenyl)-4,4-dimethyl-1-cyclohexen-1-yl]methyl]-1-piperazinyl]-N-[[3-nitro-4-[[(tetrahydro-2H-pyran-4-yl) methyl]amino]phenyl]sulfonyl]-2-(1H-pyrrolo[2,3-b]pyridin-5-yloxy)-benzamide, GDC 0199, and Venetoclax, has the structure:

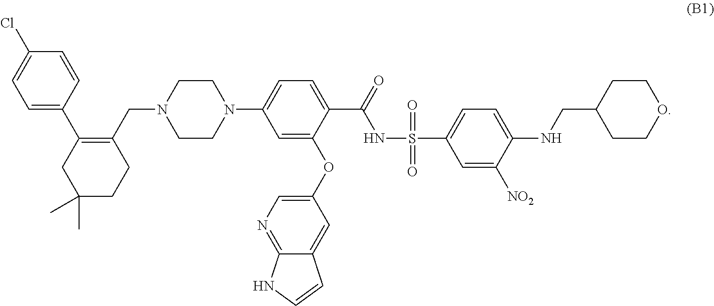 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Compound B2 may be referred as 4-(4-((4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide and has the structure:

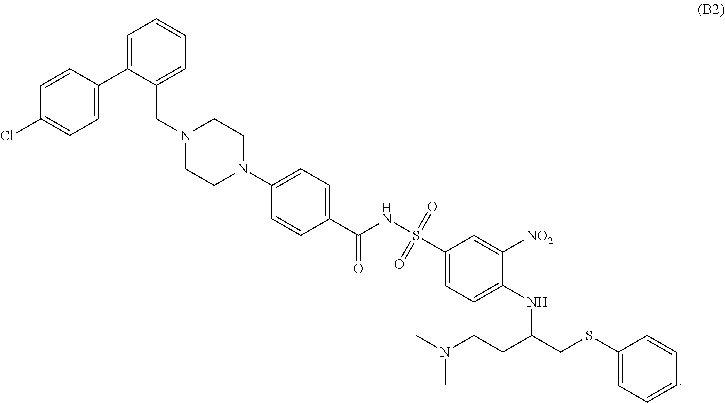 [SMILES: CN(C)CCC(CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

Compound B3 has the structure:

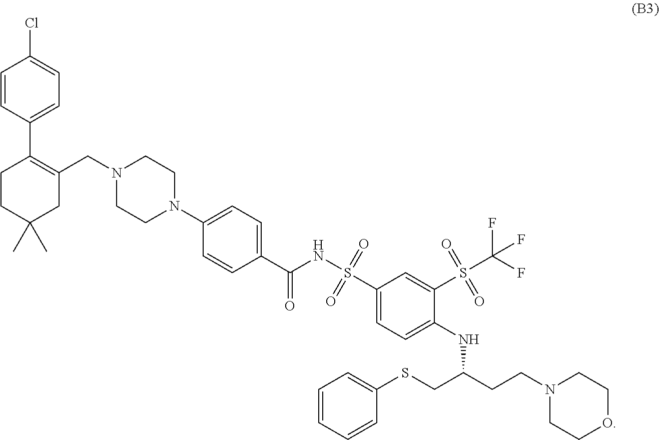 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some variations, the Syk inhibitor is Compound D1, or a pharmaceutically acceptable salt thereof, which is also known as Entospletinib, GS-9973, and 6-(1H-indazol-6-yl)-N-(4-morpholinophenyl)imidazo[1,2-a]pyrazin-8-amine.

Compound D1 has the structure:

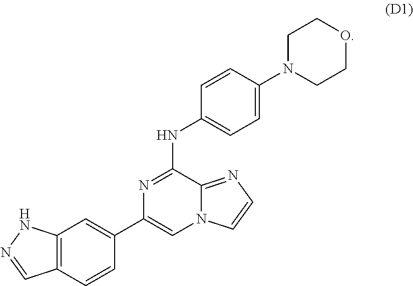 [SMILES: c1cn2cc(-c3ccc4cn[nH]c4c3)nc(Nc3ccc(N4CCOCC4)cc3)c2n1]

In some embodiments, Compound D1, or a pharmaceutically acceptable salt thereof, is used in combination with Compound A1, or a pharmaceutically acceptable salt or hydrate thereof.

In some embodiments, the Syk inhibitor is a compound of Formula II:

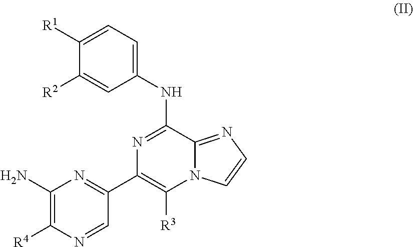

wherein
R¹ is selected from the group consisting of

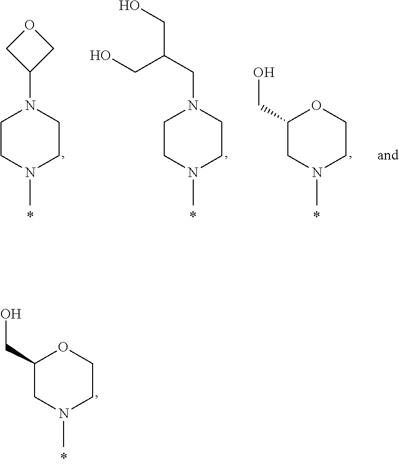

wherein * indicates the carbon atom of the indicated phenyl ring of to which R¹ is attached;
R² is H or 2-hydroxyethoxyl;
R³ is H or methyl; and
R⁴ is H or methyl.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), each of R², R³, and R⁴ is H, and R¹ is as defined above.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), R² is H, R³ is methyl, and R⁴ is H, and R¹ is as defined above.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), R² is H, R³ is H, and R⁴ is methyl, and R¹ is as defined above.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), R² is 2-hydroxyethoxyl, R³ is methyl, and R⁴ is H, and R¹ is as defined above.

Within each reference to an embodiment herein, including to a method of treatment, pharmaceutical composition, or therapeutic regimen, concerning a compound of Formula (II), it is understood that within each there are separate treatments, pharmaceutical compositions, or therapeutic regimens in which the compound of Formula (II) comprises, individually:
6-(6-amino-5-methylpyrazin-2-yl)-N-(4-(4-(oxetan-3-yl)piperazin-1-yl)phenyl)imidazo[1,2-a]pyrazin-8-amine;
6-(6-aminopyrazin-2-yl)-N-(4-(4-(oxetan-3-yl)piperazin-1-yl)phenyl)imidazo[1,2-a]pyrazin-8-amine;
(R)-(4-(4-((6-(6-aminopyrazin-2-yl)imidazo[1,2-a]pyrazin-8-yl)amino)phenyl)morpholin-2-yl)methanol;
6-(6-aminopyrazin-2-yl)-5-methyl-N-(4-(4-(oxetan-3-yl)piperazin-1-yl)phenyl)imidazo[1,2-a]pyrazin-8-amine;
2-(5-((6-(6-aminopyrazin-2-yl)imidazo[1,2-a]pyrazin-8-yl)amino)-2-(4-(oxetan-3-yl)piperazin-1-yl)phenoxy)ethanol;
2-((4-(4-((6-(6-aminopyrazin-2-yl)imidazo[1,2-a]pyrazin-8-yl)amino)phenyl)piperazin-1-yl)methyl)propane-1,3-diol; or
2-(5-((6-(6-amino-5-methylpyrazin-2-yl)imidazo[1,2-a]pyrazin-8-yl)amino)-2-(4-(oxetan-3-yl)piperazin-1-yl)phenoxy)ethanol;

or a pharmaceutically acceptable salt of co-crystal thereof.

Other Examples of SYK (spleen tyrosine kinase) inhibitors include, but are not limited to, tamatinib (R406), fostamatinib (R788), PRT062607, BAY-61-3606, NVP-QAB 205 AA, R112, R343, and those described in U.S. Pat. No. 8,450,321 (Gilead Connecticut).

For each of the embodiments disclosed herein, including the methods of treatment, pharmaceutical compositions, kits, regimens, and other uses concerning a compound of Compound A1, Compound B1, Compound B2, Compound B3, Compound C1, Compound C1(S), Compound D1 or of Formula II, including the specific examples of compounds within Formula (II) disclosed herein, it is understood that reference to Compound A1, Compound B1, Compound B2, Compound B3, Compound C1, Compound C1(S), Compound D1 or of Formula II, or a pharmaceutically acceptable salt of co-crystal thereof, also includes pharmaceutically acceptable esters, pharmaceutically acceptable solvates, hydrates, isomers (including optical isomers, racemates, or other mixtures thereof), tautomers, isotopes, polymorphs, and pharmaceutically acceptable prodrugs of such compounds.

In some embodiments, Compound B1, or a pharmaceutically acceptable salt or hydrate thereof, is used in combination with Compound A1, or a pharmaceutically acceptable salt or hydrate thereof. In other embodiments, Compound B1, or a pharmaceutically acceptable salt thereof, is used in combination with Compound A1, or a pharmaceutically acceptable salt or hydrate thereof and obinutuzumab In other embodiments, Compound B2, or a pharmaceutically acceptable salt or hydrate thereof, is used in combination with Compound A1, or a pharmaceutically acceptable salt or hydrate thereof. In other embodiments, Compound B2, or a pharmaceutically acceptable salt thereof, is used in combination with Compound A1, or a pharmaceutically acceptable salt or hydrate thereof and obinutuzumab. In other embodiments, Compound B3, or a pharmaceutically acceptable salt or hydrate thereof, is used in combination with Compound A1, or a pharmaceutically acceptable salt or hydrate thereof. In yet other embodiments, Compound B3, or a pharmaceutically acceptable salt thereof, is used in combination with Compound A1, or a pharmaceutically acceptable salt or hydrate thereof and obinutuzumab.

In some embodiments, Compound B1, or a pharmaceutically acceptable salt thereof, is used in combination with Compound C1. In other embodiments, Compound B1, or a pharmaceutically acceptable salt thereof, is used in combination with Compound C1 (S) and obinutuzumab. In other embodiments, Compound B1, or a pharmaceutically acceptable salt thereof, is used in combination with Compound C1(S) and Compound A1, or a pharmaceutically acceptable salt or hydrate thereof. In other embodiments, Compound B2, or a pharmaceutically acceptable salt thereof, is used in combination with Compound C1. In other embodiments, Compound B2, or a pharmaceutically acceptable salt thereof, is used in combination with Compound C1(S) and obinutuzumab. In other embodiments, Compound B2, or a pharmaceutically acceptable salt thereof, is used in combination with Compound C1 (S) and Compound A1, or a pharmaceutically acceptable salt or hydrate thereof. In yet other embodiments, Compound B3, or a pharmaceutically acceptable salt thereof, is used in combination with Compound C1. In other embodiments, Compound B3, or a pharmaceutically acceptable salt thereof, is used in combination with Compound C1(S) and obinutuzumab. In some embodiments, Compound B3, or a pharmaceutically acceptable salt thereof, is used in combination with Compound C1(S) and Compound A1, or a pharmaceutically acceptable salt or hydrate thereof.

In some embodiments, Compound B1, or a pharmaceutically acceptable salt or hydrate thereof, is used in combination with Compound D1, Formula II or a pharmaceutically acceptable salt or hydrate thereof. In some embodiments, Compound B1, or a pharmaceutically acceptable salt or hydrate thereof, is used in combination with Compound D1, Formula II or a pharmaceutically acceptable salt or hydrate thereof and obinutuzumab. In some embodiments, Compound B1, or a pharmaceutically acceptable salt thereof, is used in combination with Compound D1, Formula II or a pharmaceutically acceptable salt or hydrate thereof and Compound A1, or a pharmaceutically acceptable salt or hydrate thereof. In other embodiments, Compound B2, or a pharmaceutically acceptable salt or hydrate thereof, is used in combination with Compound D1, Formula II or a pharmaceutically acceptable salt or hydrate thereof. In some embodiments, Compound B2, or a pharmaceutically acceptable salt or hydrate thereof, is used in combination with Compound D1, Formula II or a pharmaceutically acceptable salt or hydrate thereof and obinutuzumab. In some embodiments, Compound B2, or a pharmaceutically acceptable salt thereof, is used in combination with Compound D1, Formula II or a pharmaceutically acceptable salt or hydrate thereof and Compound A1, or a pharmaceutically acceptable salt or hydrate thereof. In yet other embodiments, Compound B3, or a pharmaceutically acceptable salt or hydrate thereof, is used in combination with Compound D1, Formula II or a pharmaceutically acceptable salt or hydrate thereof. In some embodiments, Compound B3, or a pharmaceutically acceptable salt or hydrate thereof, is used in combination with Compound D1, Formula II or a pharmaceutically acceptable salt or hydrate thereof and obinutuzumab. In some embodiments, Compound B3, or a pharmaceutically acceptable salt thereof, is used in combination with Compound D1, Formula II or a pharmaceutically acceptable salt or hydrate thereof and Compound A1, or a pharmaceutically acceptable salt or hydrate thereof.

In some embodiments, Compound C1(S), or a pharmaceutically acceptable salt thereof, is used in combination with Compound A1 or a pharmaceutically acceptable salt or hydrate thereof. In other embodiments, Compound C1 (S), or a pharmaceutically acceptable salt thereof, is used in combination with Compound A1 or a pharmaceutically acceptable salt or hydrate thereof and obinutuzumab. In other embodiments, Compound C1(S), or a pharmaceutically acceptable salt thereof, is used in combination with Compound A1 or a pharmaceutically acceptable salt or hydrate thereof and Compound B1. In other embodiments, Compound C1(S), or a pharmaceutically acceptable salt thereof, is used in combination with Compound A1 or a pharmaceutically acceptable salt or hydrate thereof and Compound B2. In other embodiments, Compound C1(S), or a pharmaceutically acceptable salt thereof, is used in combination with Compound A1 or a pharmaceutically acceptable salt or hydrate thereof and Compound B3.

In some embodiments, Compound D1, Formula II, or a pharmaceutically acceptable salt thereof, is used in combination with Compound A1 or a pharmaceutically acceptable salt or hydrate thereof. In other embodiments, Compound D1, Formula II or a pharmaceutically acceptable salt thereof, is used in combination with Compound A1 or a pharmaceutically acceptable salt or hydrate thereof and obinutuzumab.

Compounds B1, B2 and B3 are commercially available, and their methods of synthesis are generally known in the art. For example, Compounds B1, B2 and B3 may be synthesized according to U.S. Patent Application Publication Nos. 2010/0305122, 2007/0072860, or 2007/0027135. In addition to the chemical structures, Compound B1 may also be referred to or identified as (4-(4-{[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide); Compound B2 may be referred to or identified as 4-(4-((4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide; and Compound B3 may be referred to or identified as (R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide

In one variation, the BCL-2 inhibitor is (4-(4-{[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide), or a pharmaceutically acceptable salt thereof.

In another variation, the BCL-2 inhibitor is 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]benzamide, or a pharmaceutically acceptable salt thereof.

In another variation, the BCL-2 inhibitor is 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3 [(trifluoromethyl)sulfonyl]phenyl]sulfonyl]benzamide, or a pharmaceutically acceptable salt thereof.

The compound names provided herein are named using ChemBioDraw Ultra 12.0. One skilled in the art understands that the compound may be named or identified using various commonly recognized nomenclature systems and symbols. By way of example, the compound may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC). By way of example, ABT-199 may be referred to as 1257044-40-8 by CAS, 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)benzamide by ChemBioDraw Ultra, or 4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide by IUPAC. Also, ABT-199 may also be referred to as venetoclax or Venetoclax.

Also provided herein are isotopically labeled forms of compounds detailed herein. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶C1 and ¹²⁵I. Various isotopically labeled compounds of the present disclosure, for example those into which radioactive isotopes such as ³H, ¹³C and ¹⁴C are incorporated, are provided. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g. humans). Also provided for isotopically labeled compounds described herein are any pharmaceutically acceptable salts, or hydrates, as the case may be.

In some variations, the compounds disclosed herein may be varied such that from 1 to n hydrogens attached to a carbon atom is/are replaced by deuterium, in which n is the number of hydrogens in the molecule. Such compounds may exhibit increased resistance to metabolism and are thus useful for increasing the half life of the compound when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds provided herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

The term “pharmaceutically acceptable” with respect to a substance refers to that substance which is generally regarded as safe and suitable for use without undue toxicity, irritation, allergic response, and the like, commensurate with a reasonable benefit/risk ratio. As used herein, by “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable vehicles (e.g., carriers, adjuvants, and/or other excipients) have preferably met the required standards of toxicological and manufacturing testing and/or are included on the Inactive Ingredient Guide prepared by the U.S. Food and Drug administration.

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. “Pharmaceutically acceptable salts” include, for example, salts with inorganic acids and salts with an organic acid. Examples of salts may include hydrochlorate, phosphate, diphosphate, hydrobromate, sulfate, sulfinate, nitrate, malate, maleate, fumarate, tartrate, succinate, citrate, acetate, lactate, mesylate, p-toluenesulfonate, 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Also included in this definition are ammonium and substituted or quaternized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein. Those skilled in the art will recognize various synthetic methodologies that may be used to prepare nontoxic pharmaceutically acceptable addition salts.

The terms “effective amount”, “pharmaceutically effective amount”, and “therapeutically effective amount” refer to an amount that may be effective to elicit the desired biological or medical response, including the amount of a compound that, when administered to a subject for treating a disease, is sufficient to effect such treatment for the disease. The effective amount will vary depending on the compound, the disease and its severity and the age, weight, etc., of the subject to be treated. The effective amount can include a range of amounts. A pharmaceutically effective amount includes amounts of an agent which are effective when combined with other agents.

Treatment” or “treating” is an approach for obtaining beneficial or desired results including clinical results. Beneficial or desired clinical results may include one or more of the following:

(i) decreasing one more symptoms resulting from the disease;
(ii) diminishing the extent of the disease and/or stabilizing the disease (e.g., delaying the worsening of the disease);
(iii) delaying the spread of the disease;
(iv) delaying or slowing the onset or recurrence of the disease and/or the progression of the disease;
(v) ameliorating the disease state and/or providing a remission (whether partial or total) of the disease and/or decreasing the dose of one or more other medications required to treat the disease;
(vi) increasing the quality of life;
(vii) prolonging survival;
(iix) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or
(ix) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival). Without being bound to any hypothesis or theory, the methods described herein comprising one or more agents (such as Compound A1, Compound B1, Compound C1(S), Compound D1, and/or obinutuzumab) may provide unexpected treatment benefits, including but not limited to shorter treatment periods, reducing or minimizing minimal residual disease in cancers, reducing or minimizing cancer resistance, increasing survival rates, decreasing symptoms, or slowing cancer development.

“Delaying” the development of a disease or condition means to defer, hinder, slow, retard, stabilize, and/or postpone development of the disease or condition. This delay can be of varying lengths of time, depending on the history of the disease or condition, and/or subject being treated. A method that “delays” development of a disease or condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

For use in the methods described herein, the compound of Formula (II), or a pharmaceutically acceptable salt or co-crystal thereof, may be present in a pharmaceutical composition comprising the compound of Formula (II), or a pharmaceutically acceptable salt or co-crystal thereof, and at least one pharmaceutically acceptable vehicle. Pharmaceutically acceptable vehicles may include pharmaceutically acceptable carriers, adjuvants and/or other excipients, and other ingredients can be deemed pharmaceutically acceptable insofar as they are compatible with other ingredients of the formulation and not deleterious to the recipient thereof.

The pharmaceutical compositions of the compound of Formula (II), or a pharmaceutically acceptable salt or co-crystal thereof, described herein can be manufactured using any conventional method, e.g., mixing, dissolving, granulating, dragee-making, levigating, emulsifying, encapsulating, entrapping, melt-spinning, spray-drying, or lyophilizing processes. An optimal pharmaceutical formulation can be determined by one of skill in the art depending on the route of administration and the desired dosage. Such formulations can influence the physical state, stability, rate of in vivo release, and rate of in vivo clearance of the administered agent. Depending on the condition being treated, these pharmaceutical compositions can be formulated and administered systemically or locally.

The term “carrier” refers to diluents, disintegrants, precipitation inhibitors, surfactants, glidants, binders, lubricants, and other excipients and vehicles with which the compound is administered. Carriers are generally described herein and also in “Remington's Pharmaceutical Sciences” by E. W. Martin. Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

The term “diluent” generally refers to a substance used to dilute the compound of interest prior to delivery. Diluents can also serve to stabilize compounds. Examples of diluents may include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, microcrystalline cellulose, and tribasic calcium phosphate.

The term “disintegrant” generally refers to a substance which, upon addition to a solid preparation, facilitates its break-up or disintegration after administration and permits the release of an active ingredient as efficiently as possible to allow for its rapid dissolution. Examples of disintegrants may include maize starch, sodium starch glycolate, croscarmellose sodium, crospovidone, microcrystalline cellulose, modified corn starch, sodium carboxymethyl starch, povidone, pregelatinized starch, and alginic acid.

The term “precipitation inhibitors” generally refers to a substance that prevents or inhibits precipitation of the active agent from a supersaturated solution. One example of a precipitation inhibitor includes hydroxypropylmethylcellulose (HPMC).

The term “surfactants” generally refers to a substance that lowers the surface tension between a liquid and a solid that could improve the wetting of the active agent or improve the solubility of the active agent. Examples of surfactants include poloxamer and sodium lauryl sulfate.

The term “glidant” generally refers to substances used in tablet and capsule formulations to improve flow-properties during tablet compression and to produce an anti-caking effect. Examples of glidants may include colloidal silicon dioxide, talc, fumed silica, starch, starch derivatives, and bentonite.

The term “binder” generally refers to any pharmaceutically acceptable film which can be used to bind together the active and inert components of the carrier together to maintain cohesive and discrete portions. Examples of binders may include hydroxypropylcellulose, hydroxypropylmethylcellulose, povidone, copovidone, and ethyl cellulose.

The term “lubricant” generally refers to a substance that is added to a powder blend to prevent the compacted powder mass from sticking to the equipment during the tableting or encapsulation process. A lubricant can aid the ejection of the tablet form the dies, and can improve powder flow. Examples of lubricants may include magnesium stearate, stearic acid, silica, fats, calcium stearate, polyethylene glycol, sodium stearyl fumarate, or talc; and solubilizers such as fatty acids including lauric acid, oleic acid, and C₈/C₁₀ fatty acid.

The therapeutically effective amount may vary depending on the subject, and disease or condition being treated, the weight and age of the subject, the severity of the disease or condition, and the manner of administering, which can readily be determined by one or ordinary skill in the art.

The dosing regimen of the compounds described herein, i.e. Compound A1, Compound B1, Compound B2, Compound B3, Compound C1, Compound C1(S), Compound D1, Formula II, or a pharmaceutically acceptable salt or co-crystal thereof, provided herein may vary depending upon the indication, route of administration, and severity of the condition. The dosing regimen of the compounds of Formula A1, and a Syk inhibiting compound of Formula D1 or of Formula (II), or a pharmaceutically acceptable salt or co-crystal thereof, in the methods provided herein may vary depending upon the indication, route of administration, and severity of the condition, for example. Depending on the route of administration, a suitable dose can be calculated according to body weight, body surface area, or organ size. The final dosing regimen is determined by the attending physician in view of good medical practice, considering various factors that modify the action of drugs, e.g., the specific activity of the compound, the identity and severity of the disease state, the responsiveness of the subject, the age, condition, body weight, sex, and diet of the subject, and the severity of any infection. Additional factors that can be taken into account include time and frequency of administration, drug combinations, reaction sensitivities, and tolerance/response to therapy. Further refinement of the doses appropriate for treatment involving any of the formulations mentioned herein is done routinely by the skilled practitioner without undue experimentation, especially in light of the dosing information and assays disclosed, as well as the pharmacokinetic data observed in human clinical trials. Appropriate doses can be ascertained through use of established assays for determining concentration of the agent in a body fluid or other sample together with dose response data.

The formulation and route of administration chosen may be tailored to the individual subject, the nature of the condition to be treated in the subject, and generally, the judgment of the attending practitioner.

The pharmaceutically effective amount or therapeutically effective amount of the compound of Formula A1, Formula D1, or of Formula II, or a pharmaceutically acceptable salt or co-crystal thereof, may be provided in a single dose or multiple doses to achieve the desired treatment endpoint. As used herein, “dose” refers to the total amount of an active ingredient (e.g., the compound of Formula A1, Formula D1, or of Formula II, or a pharmaceutically acceptable salt or co-crystal thereof,) to be taken each time by a subject (e.g., a human). The dose administered, for example for oral administration described above, may be administered once daily (QD), twice daily (BID), three times daily, four times daily, or more than four times daily. Similarly, the pharmaceutically effective amount or therapeutically effective amount of the compound described herein, i.e. Compound A1, Compound B1, Compound B2, Compound B3, Compound C1, Compound C1(S), Compound D1, Formula II, or a pharmaceutically acceptable salt or co-crystal thereof, may be provided in a single dose or multiple doses to achieve the desired treatment endpoint. In some embodiments, the dose of a compound of Formula A1, Formula D1, or of Formula II, or a pharmaceutically acceptable salt or co-crystal thereof, is administered once daily. In some embodiments, the dose of a compound of Formula A1, Formula D1, or of Formula II, or a pharmaceutically acceptable salt or co-crystal thereof, is administered twice daily. In some other embodiments, the dose of Compound A1, Compound B1, Compound B2, Compound B3, Compound C1, Compound C1(S), Compound D1, or a pharmaceutically acceptable salt or co-crystal thereof, is administered once daily. In some additional embodiments, the dose of Compound A1, Compound B1, Compound B2, Compound B3, Compound C1, Compound C1(S), Compound D1, or a pharmaceutically acceptable salt or co-crystal thereof, is administered twice daily.

In some embodiments, exemplary doses of the Syk inhibitor compound of Formula D1 or of Formula II, or a pharmaceutically acceptable salt or co-crystal thereof, for a human subject may be from about 1 mg to about 5000 mg, about 1 mg to about 4000 mg, about 1 mg to about 3000 mg, about 1 mg to about 2000 mg, about 2 mg to about 2000 mg, about 5 mg to about 2000 mg, about 10 mg to about 2000 mg, about 1 mg to about 1000 mg, about 2 mg to about 1000 mg, about 5 mg to about 1000 mg, about 10 mg to about 1000 mg, about 25 mg to about 1000 mg, about 50 mg to about 1000 mg, about 75 mg to about 1000 mg, about 100 mg to about 1000 mg, about 125 mg to about 1000 mg, about 150 mg to about 1000 mg, about 175 mg to about 1000 mg, about 200 mg to about 1000 mg, about 225 mg to about 1000 mg, about 250 mg to about 1000 mg, about 300 mg to about 1000 mg, about 350 mg to about 1000 mg, about 400 mg to about 1000 mg, about 450 mg to about 1000 mg, about 500 mg to about 1000 mg, about 550 mg to about 1000 mg, about 600 mg to about 1000 mg, about 650 mg to about 1000 mg, about 700 mg to about 1000 mg, about 750 mg to about 1000 mg, about 800 mg to about 1000 mg, about 850 mg to about 1000 mg, about 900 mg to about 1000 mg, about 950 mg to about 1000 mg, about 1 mg to about 750 mg, about 2 mg to about 750 mg, about 5 mg to about 750 mg, about 10 mg to about 750 mg, about 25 mg to about 750 mg, about 50 mg to about 750 mg, about 75 mg to about 750 mg, about 100 mg to about 750 mg, about 125 mg to about 750 mg, about 150 mg to about 750 mg, about 175 mg to about 750 mg, about 200 mg to about 750 mg, about 225 mg to about 750 mg, about 250 mg to about 750 mg, about 300 mg to about 750 mg, about 350 mg to about 750 mg, about 400 mg to about 750 mg, about 450 mg to about 750 mg, about 500 mg to about 750 mg, about 550 mg to about 750 mg, about 600 mg to about 750 mg, about 650 mg to about 750 mg, about 700 mg to about 750 mg, about 1 mg to about 500 mg, about 2 mg to about 500 mg, about 5 mg to about 500 mg, about 10 mg to about 500 mg, about 25 mg to about 500 mg, about 50 mg to about 500 mg, about 75 mg to about 500 mg, about 100 mg to about 500 mg, about 125 mg to about 500 mg, about 150 mg to about 500 mg, about 175 mg to about 500 mg, about 200 mg to about 500 mg, about 225 mg to about 500 mg, about 250 mg to about 500 mg, about 300 mg to about 500 mg, about 350 mg to about 500 mg, about 400 mg to about 500 mg, about 450 mg to about 500 mg, about 1 mg to about 400 mg, about 2 mg to about 400 mg, about 5 mg to about 400 mg, about 10 mg to about 400 mg, about 25 mg to about 400 mg, about 50 mg to about 400 mg, about 75 mg to about 400 mg, about 100 mg to about 400 mg, about 125 mg to about 400 mg, about 150 mg to about 400 mg, about 175 mg to about 400 mg, about 200 mg to about 400 mg, about 225 mg to about 400 mg, about 250 mg to about 400 mg, about 300 mg to about 400 mg, about 350 mg to about 400 mg, about 1 mg to about 300 mg, about 2 mg to about 300 mg, about 5 mg to about 300 mg, about 10 mg to about 300 mg, about 25 mg to about 300 mg, about 50 mg to about 300 mg, about 75 mg to about 300 mg, about 100 mg to about 300 mg, about 125 mg to about 300 mg, about 150 mg to about 300 mg, about 175 mg to about 300 mg, about 200 mg to about 300 mg, about 225 mg to about 300 mg, about 250 mg to about 300 mg, about 1 mg to about 250 mg, about 2 mg to about 250 mg, about 5 mg to about 250 mg, about 10 mg to about 250 mg, about 25 mg to about 250 mg, about 50 mg to about 250 mg, about 75 mg to about 250 mg, about 100 mg to about 250 mg, about 125 mg to about 250 mg, about 150 mg to about 250 mg, about 175 mg to about 250 mg, about 200 mg to about 250 mg, about 225 mg to about 250 mg, about 1 mg to about 225 mg, about 2 mg to about 225 mg, about 5 mg to about 225 mg, about 10 mg to about 225 mg, about 25 mg to about 225 mg, about 50 mg to about 225 mg, about 75 mg to about 225 mg, about 100 mg to about 225 mg, about 125 mg to about 225 mg, about 150 mg to about 225 mg, about 175 mg to about 225 mg, about 200 mg to about 225 mg, about 1 mg to about 200 mg, about 2 mg to about 200 mg, about 5 mg to about 200 mg, about 10 mg to about 200 mg, about 25 mg to about 200 mg, about 50 mg to about 200 mg, about 75 mg to about 200 mg, about 100 mg to about 200 mg, about 125 mg to about 200 mg, about 150 mg to about 200 mg, about 175 mg to about 200 mg, about 180 mg to about 200 mg, about 1 mg to about 175 mg, about 2 mg to about 175 mg, about 5 mg to about 175 mg, about 10 mg to about 175 mg, about 25 mg to about 175 mg, about 50 mg to about 175 mg, about 75 mg to about 175 mg, about 100 mg to about 175 mg, about 125 mg to about 175 mg, about 150 mg to about 175 mg, about 1 mg to about 150 mg, about 2 mg to about 150 mg, about 5 mg to about 150 mg, about 10 mg to about 150 mg, about 25 mg to about 150 mg, about 50 mg to about 150 mg, about 75 mg to about 150 mg, about 100 mg to about 150 mg, about 125 mg to about 150 mg, about 1 mg to about 125 mg, about 2 mg to about 125 mg, about 5 mg to about 125 mg, about 10 mg to about 125 mg, about 25 mg to about 125 mg, about 50 mg to about 125 mg, about 75 mg to about 125 mg, about 100 mg to about 125 mg, about 1 mg to about 100 mg, about 2 mg to about 100 mg, about 5 mg to about 100 mg, about 10 mg to about 100 mg, about 25 mg to about 100 mg, about 50 mg to about 100 mg, about 60 mg to about 100 mg, or about 75 mg to about 100 mg.

In some embodiments, exemplary doses of the Syk inhibiting compound of Formula D1 or of Formula II, or a pharmaceutically acceptable salt or co-crystal thereof, for a human subject may be about 1 mg, about 2 mg, about 5 mg, about 10 mg, about 15 mg, about 20 mg, about 25 mg, about 30 mg, about 35 mg, about 40 mg, about 45 mg, about 50 mg, about 60 mg, about 65 mg, about 70 mg, about 75 mg, about 100 mg, about 125 mg, about 150 mg, about 175 mg, about 180 mg, about 190 mg, about 200 mg, about 225 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1200 mg, about 1400 mg, about 1600 mg, about 1800 mg, about 2000 mg, about 2200 mg, about 2400 mg, about 2600 mg, about 2800 mg, about 3000 mg, about 3200 mg, about 3400 mg, about 3600 mg, about 3800 mg, about 4000 mg, about 4200 mg, about 4400 mg, about 4600 mg, about 4800 mg, or about 5000 mg. In certain embodiment, SYK inhibiting compound (such as Compound D1) is administered at the dose of 200 mg or 400 mg once daily. In one embodiment, SYK inhibiting compound (such as Compound D1) is administered at the dose of 200 mg or 400 mg twice daily.

In other embodiments, the methods provided comprise administering the doses of Compound A1, Compound B1, Compound B2, Compound B3, Compound C1, Compound C1(S), Compound D1, Formula II, or a pharmaceutically acceptable salt or co-crystal thereof, at which clinical efficacy is achieved or reducing the doses by increments to a level at which efficacy can be maintained. In other embodiments, the methods provided comprise continuing to treat the subject (e.g., a human) by administering the doses of the compounds of Formula A1, Formula D1, or of Formula II, or a pharmaceutically acceptable salt or co-crystal thereof, at which clinical efficacy is achieved or reducing the doses by increments to a level at which efficacy can be maintained. In some embodiments, the methods provided comprise administering to the subject (e.g., a human) an initial daily dose of 50 mg to about 500 mg or the Syk inhibiting compound of Formula D1 or of Formula II, or a pharmaceutically acceptable salt or co-crystal thereof, or in an alternative embodiment 100 mg to 1000 mg of the compound of Formula A1 or of Formula II, or a pharmaceutically acceptable salt or co-crystal thereof, and administering subsequent daily doses of the compounds of Formula D1 or of Formula II, or a pharmaceutically acceptable salt or co-crystal thereof, over at least 6 days, wherein each subsequent daily dose is increased by 25 mg to 300 mg, or by 50 mg to about 400 mg. Thus, it should also be understood that the dose of the Syk inhibiting compound of Formula D1 or of Formula II, or a pharmaceutically acceptable salt or co-crystal thereof, may be increased by increments until clinical efficacy is achieved. Increments of about 10 mg, about 25 mg, about 50 mg, about 100 mg, or about 125 mg, or about 150 mg, or about 200 mg, or about 250 mg, or about 300 mg can be used to increase the dose. The dose can be increased daily, every other day, two, three, four, five or six times per week, or once per week. Initial doses of a Syk inhibiting compound of Formula D1 or of Formula II, or a pharmaceutically acceptable salt or co-crystal thereof, may be selected from 250 mg, 300 mg, 350 mg, 400 mg, 450 mg, or 500 mg, each administered once, twice, or three times daily. Additionally, initial doses of a BCL inhibiting compound of Compound B1, Compound B2, and Compound B3, or a pharmaceutically acceptable salt or co-crystal thereof, may be selected from 50 mg, 100 mg, 200 mg, 300 mg, 400 mg, or 420 mg, each administered once, twice, or three times daily. Also, initial doses of a PI3K-δ inhibiting compound of Compound A1 and Compound A1(S), or a pharmaceutically acceptable salt or co-crystal thereof, may be selected from 50 mg, 100 mg, 150, 200 mg, or 300 mg, each administered once, twice, or three times daily. Moreover, initial doses of a BTK inhibiting compound of Compound A1, or a pharmaceutically acceptable salt or co-crystal thereof, may be selected from 20 mg, 40 mg, 80 mg, 150 mg, 200 mg, or 250 mg, each administered once, twice, or three times daily.

The frequency of dosing will depend on the pharmacokinetic parameters of the compound administered, the route of administration, and the particular disease treated. The dose and frequency of dosing may also depend on pharmacokinetic and pharmacodynamic, as well as toxicity and therapeutic efficiency data. For example, pharmacokinetic and pharmacodynamic information about the Syk inhibiting compound of Formula D1 or of Formula (II), or a pharmaceutically acceptable salt or co-crystal thereof, can be collected through preclinical in vitro and in vivo studies, later confirmed in humans during the course of clinical trials. Thus, for the compound of Formula D1 or of Formula (II), or a pharmaceutically acceptable salt or co-crystal thereof, used in the methods provided herein, a therapeutically effective dose can be estimated initially from biochemical and/or cell-based assays. Similar, for Compound A1, Compound B1, Compound B2, Compound B3, Compound C1, Compound C1(S), or a pharmaceutically acceptable salt or co-crystal thereof, used in the methods provided herein, a therapeutically effective dose may be estimated initially from biochemical and/or cell-based assays. Then, dosage can be formulated in animal models to achieve a desirable circulating concentration range that modulates Syk expression or activity. As human studies are conducted further information will emerge regarding the appropriate dosage levels and duration of treatment for various diseases and conditions.

Toxicity and therapeutic efficacy of the compound of Formula D1 or of Formula (II), or a pharmaceutically acceptable salt or co-crystal thereof, can be determined by standard pharmaceutical procedures in cell cultures or experimental animals, e.g., for determining the LD₅₀ (the dose lethal to 50% of the population) and the ED₅₀ (the dose therapeutically effective in 50% of the population). Also, toxicity and therapeutic efficacy of Compound A1, Compound B1, Compound B2, Compound B3, Compound C1, Compound C1(S), or a pharmaceutically acceptable salt or co-crystal thereof, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index”, which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosage for human use. The doses of such compounds lies preferably within a range of circulating concentrations that include the ED₅₀ with little or no toxicity.

Compositions (including, for example, formulations and unit dosages) comprising a compound of Formula A1, Formula D1, or Formula (II), or a pharmaceutically acceptable salt or co-crystal thereof, can be prepared and placed in an appropriate container, and labeled for treatment of an indicated condition. Accordingly, provided is also an article of manufacture, such as a container comprising a unit dosage form of the compound of Formula A1 and a unit dosage form of a compound of Formula D1 or Formula II, or a pharmaceutically acceptable salt or co-crystal thereof, and a label containing instructions for use of the compounds. In some embodiments, the article of manufacture is a container comprising a unit dosage form of a Syk inhibiting compound of Formula D1 or Formula II, or a pharmaceutically acceptable salt or co-crystal thereof, and at least one pharmaceutically acceptable vehicle and a unit dosage form of a compound of Formula A1, or a pharmaceutically acceptable salt or co-crystal thereof, and at least one pharmaceutically acceptable vehicle. Also, compositions (including, for example, formulations and unit dosages) comprising Compound B1, Compound B2, Compound B3, Compound C1, Compound C1(S), or a pharmaceutically acceptable salt or co-crystal thereof, can be prepared and placed in an appropriate container, and labeled for treatment of an indicated condition. Accordingly, provided is also an article of manufacture, such as a container comprising a unit dosage form of Compound B1, Compound B2, Compound B3, Compound C1, Compound C1(S), or a pharmaceutically acceptable salt or co-crystal thereof, and a label containing instructions for use of the compounds.

The article of manufacture may be a bottle, vial, ampoule, single-use disposable applicator, or the like, containing the pharmaceutical composition provided in the present disclosure. The container may be formed from a variety of materials, such as glass or plastic and in one aspect also contains a label on, or associated with, the container which indicates directions for use in the treatment of cancer or inflammatory conditions. It should be understood that the active ingredient may be packaged in any material capable of improving chemical and physical stability, such as an aluminum foil bag. In some embodiments, diseases or conditions indicated on the label can include, for example, treatment of cancer.

### Methods of Treatment

The BTK and BCL-2 inhibitors described herein may be used in a combination therapy. Accordingly, provided herein is a method for treating cancer in a human in need thereof, comprising administering to the human a therapeutically effective amount of a BTK inhibitor and a therapeutically effective amount of a BCL-2 inhibitor, as described herein.

The PI3K and BCL-2 inhibitors described herein may be used in a combination therapy. Accordingly, provided herein is a method for treating cancer in a human in need thereof, comprising administering to the human a therapeutically effective amount of a PI3K inhibitor and a therapeutically effective amount of a BCL-2 inhibitor, as described herein. In some embodiment the combination of Btk and PI3K delta inhibitors may be further combined with obinutuzumab. In other embodiments, the combination of Btk and PI3K delta inhibitors may be further combined with ABT-199. In one embodiment, Compound C1(S) may be combined with ABT-199 according to the methods described herein. In some other embodiment the combination of Compound A1 and Compound C1(S) may be further combined with obinutuzumab. In other embodiments, the combination of Compound A1 and Compound C1(S) may be further combined with ABT-199.

The Btk and Syk inhibitors described herein may be used in a combination therapy. Accordingly, provided herein is a method for treating cancer in a human in need thereof, comprising administering to the human a therapeutically effective amount of a BTK inhibitor and a therapeutically effective amount of a Syk inhibitor, as described herein. In some embodiment the combination of Btk and Syk inhibitors may be further combined with obinutuzumab. In other embodiments, the combination of Btk and Syk inhibitors may further combined with ABT-199. In one embodiment, Compound A1 may be combined with Compound D1 according to the methods described herein. In certain embodiment the combination of Compound A1 and Compound D1 may be further combined with obinutuzumab. In certain other embodiments, the combination of Compound A1 and Compound D1 may further combined with ABT-199.

The Btk (may also be referred as BTK inhibitor) as described herein may be used in combination with obinutuzumab and/or ABT-199. The PI3K delta (may also be referred to as PI3K-delta, PI3K-6, or PI3Kδ) inhibitor as described herein may be used in combination with obinutuzumab and/or ABT-199. The Syk (may also be referred to as SYK) inhibitor as described herein may be used in combination with obinutuzumab and/or ABT-199. In one embodiment, Compound A1 may be used in combination with obinutuzumab and/or ABT-199. In other embodiment, Compound C1(S) may be used in combination with obinutuzumab and/or ABT-199. In additional embodiment, Compound D1 may be used in combination with obinutuzumab and/or ABT-199.

### Cancer

In some embodiments, the cancer is a B-cell cancer. In some embodiments, the cancer is carcinoma, sarcoma, melanoma, lymphoma or leukemia. In other embodiments, the cancer is a hematologic malignancy. In some embodiments, the cancer is leukemia (e.g., chronic lymphocytic leukemia), lymphoma (e.g., non-Hodgkin's lymphoma), or multiple myeloma. In other embodiments, the cancer is a solid tumor.

In some embodiments, the cancer is carcinoma, sarcoma, melanoma, lymphoma or leukemia. In other embodiments, the cancer is a hematologic malignancy. In some embodiments, the cancer is leukemia (e.g., chronic lymphocytic leukemia), lymphoma (e.g., non-Hodgkin's lymphoma), or multiple myeloma. In other embodiments, the cancer is a solid tumor.

In some variations, the cancer is small lymphocytic lymphoma, non-Hodgkin's lymphoma, indolent non-Hodgkin's lymphoma (iNHL), refractory iNHL, mantle cell lymphoma, follicular lymphoma, lymphoplasmacytic lymphoma, marginal zone lymphoma, immunoblastic large cell lymphoma, lymphoblastic lymphoma, Splenic marginal zone B-cell lymphoma (+/−villous lymphocytes), nodal marginal zone lymphoma (+/−monocytoid B-cells), extranodal marginal zone B-cell lymphoma of mucosa-associated lymphoid tissue type, cutaneous T-cell lymphoma, extranodal T-cell lymphoma, anaplastic large cell lymphoma, angioimmunoblastic T-cell lymphoma, mycosis fungoides, B-cell lymphoma, diffuse large B-cell lymphoma, mediastinal large B-cell lymphoma, intravascular large B-cell lymphoma, primary effusion lymphoma, small non-cleaved cell lymphoma, Burkitt's lymphoma, multiple myeloma, plasmacytoma, acute lymphocytic leukemia, T-cell acute lymphoblastic leukemia, B-cell acute lymphoblastic leukemia, B-cell prolymphocytic leukemia, acute myeloid leukemia, chronic lymphocytic leukemia, juvenile myelomonocytic leukemia, minimal residual disease, hairy cell leukemia, primary myelofibrosis, secondary myelofibrosis, chronic myeloid leukemia, myelodysplastic syndrome, myeloproliferative disease, or Waldestrom's macroglobulinemia. In some variations, the cancer is minimal residual disease (MRD). In certain variation, the MRD may be in lymphoma, leukemia, non-Hodgkin's lymphoma or indolent non-Hodgkin's lymphoma (iNHL), small lymphocytic lymphoma (SLL), chronic lymphocytic leukemia (CLL), follicular lymphoma (FL), Waldestrom's macroglobulinemia (WM), or diffuse large B-cell lymphoma (DLBCL).

In some variations, the cancer is non-Hodgkin's lymphoma, indolent non-Hodgkin's lymphoma (iNHL), or refractory iNHL. In some variations, the cancer is non-Hodgkin's lymphoma or indolent non-Hodgkin's lymphoma (iNHL).

In some variations, the cancer is small lymphocytic lymphoma (SLL), mantle cell lymphoma, follicular lymphoma, diffuse large B-cell lymphoma, chronic lymphocytic leukemia, or Waldestrom's macroglobulinemia.

In some variations, the cancer is small lymphocytic lymphoma (SLL), follicular lymphoma, or chronic lymphocytic leukemia. In certain variations, the cancer is chronic lymphocytic leukemia (CLL).

In some embodiments, the cancer is a B-cell malignancy. In some embodiments, the B-cell malignancy is a B-cell lymphoma or a B-cell leukemia. In some variations, the B-cell malignancy is follicular lymphoma (FL), marginal zone lymphoma (MZL), small lymphocytic lymphoma (SLL), chronic lymphocytic leukemia (CLL), mantle cell lymphoma (MCL), Waldenstrom Macroglobulinemia (WM), non-germinal center B-cell lymphoma (GCB), or diffuse large B-cell lymphoma (DLBCL).

In some variations, the B-cell malignancy is diffuse large B-cell lymphoma (DLBCL). In one variation, the DLBCL is activated B-cell like diffuse large B-cell lymphoma (ABC-DLBCL). In another variation, the DLBCL is germinal center B-cell like diffuse large B-cell lymphoma (GCB-DLBCL).

In other variations, the B-cell malignancy is chronic lymphocytic leukemia (CLL). In other variations, the B-cell malignancy is mantle cell lymphoma (MCL). In yet other variations, the B-cell malignancy is Waldenstrom Macroglobulinemia (WM).

In some variations, the B-cell malignancy is indolent non-Hodgkin's lymphoma.

In other variations, the cancer is pancreatic cancer, urological cancer, bladder cancer, colorectal cancer, colon cancer, breast cancer, prostate cancer, renal cancer, hepatocellular cancer, thyroid cancer, gall bladder cancer, lung cancer (e.g. non-small cell lung cancer, small-cell lung cancer), ovarian cancer, cervical cancer, gastric cancer, endometrial cancer, esophageal cancer, head and neck cancer, melanoma, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, Kaposi's sarcomas, myxoid carcinoma, round cell carcinoma, squamous cell carcinomas, esophageal squamous cell carcinomas, oral carcinomas, cancers of the adrenal cortex, or ACTH-producing tumors. In some variations, the cancer is pancreatic cancer.

In some variation, the cancer is chronic lymphocytic leukemia (CLL), non-Hodgkin's lymphoma or indolent non-Hodgkin's lymphoma (iNHL), diffuse large B-cell lymphoma (DLBCL), or minimal residual disease (MRD). In other variation, the cancer is CLL, iNHL, DLBCL, MRD in iNHL, CLL, or DLBCL.

### Subject

The human in need thereof may be an individual who has or is suspected of having a cancer. In some of variations, the human is at risk of developing a cancer (e.g., a human who is genetically or otherwise predisposed to developing a cancer) and who has or has not been diagnosed with the cancer. As used herein, an “at risk” subject is a subject who is at risk of developing cancer (e.g., a cancer, a hematologic malignancy or a B-cell malignancy). The subject may or may not have detectable disease, and may or may not have displayed detectable disease prior to the treatment methods described herein. An at risk subject may have one or more so-called risk factors, which are measurable parameters that correlate with development of cancer, such as described herein. A subject having one or more of these risk factors has a higher probability of developing cancer than an individual without these risk factor(s).

These risk factors may include, for example, age, sex, race, diet, history of previous disease, presence of precursor disease, genetic (e.g., hereditary) considerations, and environmental exposure. In some embodiments, a human at risk for cancer includes, for example, a human whose relatives have experienced this disease, and those whose risk is determined by analysis of genetic or biochemical markers. Prior history of having cancer may also be a risk factor for instances of cancer recurrence.

In some embodiments, provided herein is a method for treating a human who exhibits one or more symptoms associated with cancer (e.g., a cancer, a hematologic malignancy or a B-cell malignancy). In some embodiments, the human is at an early stage of cancer. In other embodiments, the human is at an advanced stage of cancer.

In some embodiments, provided herein is a method for treating a human who is undergoing one or more standard therapies for treating cancer (e.g., a hematologic malignancy), such as chemotherapy, radiotherapy, immunotherapy, and/or surgery. Thus, in some foregoing embodiments, the combination of a BTK inhibitor and a BCL-2 inhibitor, as described herein, may be administered before, during, or after administration of chemotherapy, radiotherapy, immunotherapy, and/or surgery.

In some embodiments, provided herein is a method for treating a human who is undergoing one or more standard therapies for treating cancer (e.g., a cancer, a hematologic malignancy or a B-cell malignancy), such as chemotherapy, radiotherapy, immunotherapy, and/or surgery. Thus, in some foregoing embodiments, the combination of a PI3K inhibitor and a BCL-2 inhibitor, as described herein, may be administered before, during, or after administration of chemotherapy, radiotherapy, immunotherapy, and/or surgery.

In some embodiments, provided herein is a method for treating a human who is undergoing one or more standard therapies for treating cancer (e.g., a hematologic malignancy), such as chemotherapy, radiotherapy, immunotherapy, and/or surgery. Thus, in some foregoing embodiments, the combination of a Btk inhibitor and a Syk inhibitor, as described herein, may be administered before, during, or after administration of chemotherapy, radiotherapy, immunotherapy, and/or surgery.

In another aspect, provided herein is a method for treating a human who is “refractory” to a cancer treatment or who is in “relapse” after treatment for cancer (e.g., a hematologic malignancy). A subject “refractory” to an anti-cancer therapy means they do not respond to the particular treatment, also referred to as resistant. The cancer may be resistant to treatment from the beginning of treatment, or may become resistant during the course of treatment, for example after the treatment has shown some effect on the cancer, but not enough to be considered a remission or partial remission. A subject in “relapse” means that the cancer has returned or the signs and symptoms of cancer have returned after a period of improvement, e.g., after a treatment has shown effective reduction in the cancer, such as after a subject is in remission or partial remission.

In some variations, the human is (i) refractory to at least one anti-cancer therapy, or (ii) in relapse after treatment with at least one anti-cancer therapy, or both (i) and (ii). In some embodiments, the human is refractory to at least two, at least three, or at least four anti-cancer therapies (including, for example, standard or experimental chemotherapies).

In another aspect, provided is a method for sensitizing a human who is (i) refractory to at least one chemotherapy treatment, or (ii) in relapse after treatment with chemotherapy, or both (i) and (ii), wherein the method comprises administering a BTK inhibitor in combination with a BCL-2 inhibitor, as described herein, to the human. A human who is sensitized is a human who is responsive to the treatment involving administration of a BTK inhibitor in combination with a BCL-2 inhibitor, as described herein, or who has not developed resistance to such treatment.

In another aspect, provided is a method for sensitizing a human who is (i) refractory to at least one chemotherapy treatment, or (ii) in relapse after treatment with chemotherapy, or both (i) and (ii), wherein the method comprises administering a PI3K inhibitor in combination with a BCL-2 inhibitor, as described herein, to the human. A human who is sensitized is a human who is responsive to the treatment involving administration of a PI3K inhibitor in combination with a BCL-2 inhibitor, as described herein, or who has not developed resistance to such treatment.

In another aspect, provided is a method for sensitizing a human who is (i) refractory to at least one chemotherapy treatment, or (ii) in relapse after treatment with chemotherapy, or both (i) and (ii), wherein the method comprises administering a Btk inhibitor in combination with a Syk inhibitor, as described herein, to the human. A human who is sensitized is a human who is responsive to the treatment involving administration of a Btk inhibitor in combination with a Syk inhibitor, as described herein, or who has not developed resistance to such treatment.

In another aspect, provided herein is a method for treating a human for a cancer, with comorbidity, wherein the treatment is also effective in treating the comorbidity. A “comorbidity” to cancer is a disease that occurs at the same time as the cancer.

In certain variations, the cancer treatment or anti-cancer therapy is one or more of the following regimens:
a) fludarabine (FLUDARA®);
b) rituximab (RITUXAN®);
c) rituximab combined with fludarabine (sometimes abbreviated as FR);
d) cyclophosphamide (CYTOXAN®) combined with fludarabine;
e) cyclophosphamide combined with rituximab and fludarabine (sometimes abbreviated as FCR);
f) cyclophosphamide combined with vincristine and prednisone (sometimes abbreviated as CVP);
g) cyclophosphamide combined with vincristine, prednisone, and rituximab;
h) combination of cyclophosphamide, doxorubicin, vincristine (ONCOVIN®), and prednisone (sometimes referred to as CHOP);
i) chlorambucil combined with prednisone, rituximab, obinutuzumab, or ofatumumab;
j) pentostatin combined with cyclophosphamide and rituximab (sometimes abbreviated as PCR);
k) bendamustine (TREANDA®) combined with rituximab (sometimes abbreviated as BR);
l) alemtuzumab (CAMPATH®);
m) fludarabine plus cyclophosphamide, bendamustine, or chlorambucil; and
n) fludarabine plus cyclophosphamide, bendamustine, or chlorambucil, combined with an anti-CD20 antibody such as rituximab, ofatumumab, or obinutuzumab.

### Therapeutically Effective Amounts

In some variations, a therapeutically effective amount refers to an amount that is sufficient to effect treatment, as defined below, when administered to a subject (e.g., a human) in need of such treatment. The therapeutically effective amount will vary depending upon the subject and disease condition being treated, the weight and age of the subject, the severity of the disease condition, the manner of administration and the like, which can readily be determined by one of ordinary skill in the art. For example, in one variation, a therapeutically effective amount of Compound A1, or a pharmaceutically acceptable salt or hydrate thereof, is an amount sufficient to modulate BTK expression, and thereby treat a human suffering an indication, or to ameliorate or alleviate the existing symptoms of the indication. In one variation, a therapeutically effective amount of Compound B1, Compound B2 or Compound B3, or a pharmaceutically acceptable salt thereof, is an amount sufficient to modulate activity of anti-apoptotic BCL-2 proteins, and thereby treat a human suffering an indication, or to ameliorate or alleviate the existing symptoms of the indication.

In another variation, the therapeutically effective amount of the BTK inhibitor, such as Compound A1, or a pharmaceutically acceptable salt or hydrate thereof, may be an amount sufficient to decrease a symptom of a disease or condition responsive to inhibition of BTK activity. In another variation, the therapeutically effective amount of the BCL-2 inhibitor, such as Compound B1, Compound B2 or Compound B3, or a pharmaceutically acceptable salt thereof, may be an amount sufficient to decrease activity of anti-apoptotic BCL-2 proteins.

In one variation, the therapeutically effective amount of the BTK inhibitor is a dose corresponding to 1 nmol to 10,000 nmol of the BTK inhibitor used in an apoptosis assay run with 10% serum which approximately relates to a blood plasma concentration of 500 nmol to 2500 nmol of the BTK inhibitor. In one variation, the therapeutically effective amount of the BCL-2 inhibitor is a dose corresponding to 1 nmol to 200 nmol of the BCL-2 inhibitor used in an apoptosis assay run with 10% serum. Specific examples include 3 nM, 5 nM, 10 nM, 20 nM and 30 nM concentrations when combined with a BCL-2 inhibitor.

The therapeutically effective amount of the BTK and BCL-2 inhibitors may also be determined based on data obtained from assays known in the art, including for example, the apoptosis assay described in Example 1 below. In one variation, the therapeutically effective amount of the BTK inhibitor in a human is a dose of from about 1 mg to about 200 mg. In another embodiment the BTK in a human is administered at a dose of from about 10 mg to about 200 mg. In another embodiment the BTK in a human is administered at a dose of from about 20 mg to about 160 mg. In other separate embodiments the BTK inhibitor is administered to a human at a dose of: a) from about 10 mg to about 100 mg, b) from about 50 mg to about 175 mg, c) from about 20 mg to about 150 mg, d) from about 75 mg to about 100 mg, and e) from about 100 mg to about 200 mg. Individual doses of the BTK inhibitor that may be administered to a human in need thereof include individual doses of 1 mg, 5 mg, 10 mg, 20 mg, 30 mg, 40 mg, 50 mg, 60 mg, 70 mg, 75 mg, 80 mg, 901 mg, 100 mg, 110 mg, 120 mg, 130 mg, 140 mg, 150 mg, 160 mg, 170 mg, 175 mg, and 200 mg. The doses of the BTK inhibitor may be administered as determined by a medical professional and may be administered once daily or may be delivered twice daily, three times daily, or four times daily. In one embodiment, the method of the present application comprises administering Compound A1 or a pharmaceutically acceptable hydrochloride salt thereof at a dose of 20 mg, 40 mg, 75 mg, 80 mg, 150 mg, or 200 mg daily.

In one variation, the therapeutically effective amount of the BCL-2 inhibitor is a dose corresponding to 1 nmol to 200 nmol of the BCL-2 inhibitor used in an apoptosis assay run with 10% serum.

In another variation, the BTK inhibitor, such as Compound A1, or a pharmaceutically acceptable salt or hydrate thereof, is administered to the human at a dose resulting in about 50%, about 55%, about 60%, about 65%, about 70%, about 75%, about 80%, about 90%, about 95%, or about 99% BTK target inhibition. In another variation, the BCL-2 inhibitor, such as Compound B1, Compound B2 or Compound B3, or a pharmaceutically acceptable salt thereof, is administered to the human at a dose resulting in about 50%, about 55%, about 60%, about 65%, about 70%, about 75%, about 80%, about 90%, about 95%, or about 99% BCL-2 target inhibition.

In some variations, the BTK inhibitor, such as Compound A1, or a pharmaceutically acceptable salt or hydrate thereof, is administered to the human at a dose between 40 mg and 1200 mg, between 40 mg and 800 mg, between 40 mg and 600 mg, between 40 mg and 40 mg, about 100 mg, about 100 mg, about 200 mg, about 300 mg, about 400 mg, about 500 mg, about 600 mg, about 700 mg, or about 800 mg. In some variations, the BCL-2 inhibitor, such as Compound B1, Compound B2 or Compound B3, or a pharmaceutically acceptable salt thereof, is administered to the human at a dose between 20 to 600 mg, between 20 to 400 mg, between 20 to 200 mg, about 20 mg, about 50 mg, about 100 mg, about 200 mg, about 300 mg, about 400 mg, about 500 mg, about 600 mg, about 700 mg, or about 800 mg.

The therapeutically effective amount of the BTK and BCL-2 inhibitors may be provided in a single dose or multiple doses to achieve the desired treatment endpoint. As used herein, “dose” refers to the total amount of an active ingredient to be taken each time by a human. The dose administered, for example for oral administration described above, may be administered once weekly, once daily (QD), twice daily (BID), three times daily, four times daily, or more than four times daily. In some embodiments, the BTK and/or the BCL-2 inhibitors may be administered once daily. In some embodiments, the BTK and/or the BCL-2 inhibitors may be administered twice daily. In some embodiments, the BCL-2 inhibitors may be administered once weekly or with a frequency that can vary between daily, every other day, once every 5 days, daily for 1, 2, 3, 4, 5, 6 or 7 days and then weekly or with a regimen that can combine these different frequencies and doses to result in a final dose and regimen that is tolerated and efficacious.

In some variations, a therapeutically effective amount refers to an amount that is sufficient to effect treatment, as defined below, when administered to a subject (e.g., a human) in need of such treatment. The therapeutically effective amount will vary depending upon the subject and disease condition being treated, the weight and age of the subject, the severity of the disease condition, the manner of administration and the like, which can readily be determined by one of ordinary skill in the art. For example, in one variation, a therapeutically effective amount of Compound C1, or a pharmaceutically acceptable salt or hydrate thereof, is an amount sufficient to modulate PI3K expression, and thereby treat a human suffering an indication, or to ameliorate or alleviate the existing symptoms of the indication. In one variation, a therapeutically effective amount of Compound B1, Compound B2 or Compound B3, or a pharmaceutically acceptable salt thereof, is an amount sufficient to modulate activity of anti-apoptotic BCL-2 proteins, and thereby treat a human suffering an indication, or to ameliorate or alleviate the existing symptoms of the indication.

In another variation, the therapeutically effective amount of the PI3K inhibitor, such as Compound C1, or a pharmaceutically acceptable salt or hydrate thereof, may be an amount sufficient to decrease a symptom of a disease or condition responsive to inhibition of PI3K activity. In another variation, the therapeutically effective amount of the BCL-2 inhibitor, such as Compound B1, Compound B2 or Compound B3, or a pharmaceutically acceptable salt thereof, may be an amount sufficient to decrease activity of anti-apoptotic BCL-2 proteins.

The therapeutically effective amount of the PI3K and BCL-2 inhibitors may also be determined based on data obtained from assays known in the art, including for example, the apoptosis assay described in Example 2 below. In one variation, the therapeutically effective amount of the PI3K inhibitor is a dose corresponding to 30 nmol to 480 nmol of the PI3K inhibitor used in an apoptosis assay run with 10% serum. In one variation, the therapeutically effective amount of the BCL-2 inhibitor is a dose corresponding to 1 nmol to 200 nmol of the BCL-2 inhibitor used in an apoptosis assay run with 10% serum. In one variation, the therapeutically effective amount of the BCL-2 inhibitor is a dose corresponding to 3, 10 or 30 nmol of the BCL-2 inhibitor used in an apoptosis assay run with 10% serum.

In another variation, the PI3K inhibitor, such as Compound C1, or a pharmaceutically acceptable salt or hydrate thereof, is administered to the human at a dose resulting in about 50%, about 55%, about 60%, about 65%, about 70%, about 75%, about 80%, about 90%, about 95%, or about 99% PI3K target inhibition. In some variations, the PI3K inhibitor, such as Compound C1, or a pharmaceutically acceptable salt or hydrate thereof, is administered to the human at a dose resulting in less than about 50% PI3K target inhibition. In certain variations, the PI3K inhibitor, such as Compound C1, or a pharmaceutically acceptable salt or hydrate thereof, is administered to the human at a dose resulting in about 25% to about 50%, about 30% to about 50%, or about 40% to about 50% PI3K target inhibition. In another variation, the BCL-2 inhibitor, such as Compound B1, Compound B2 or Compound B3, or a pharmaceutically acceptable salt thereof, is administered to the human at a dose resulting in about 50%, about 55%, about 60%, about 65%, about 70%, about 75%, about 80%, about 90%, about 95%, or about 99% BCL-2 target inhibition. In another variation, the BCL-2 inhibitor, such as Compound B1, Compound B2 or Compound B3, or a pharmaceutically acceptable salt thereof, is administered to the human at a dose resulting in less than about 50% BCL-2 target inhibition. In certain variations, the BCL-2 inhibitor, such as Compound B1, Compound B2 or Compound B3, or a pharmaceutically acceptable salt thereof, is administered to the human at a dose resulting in about 25% to about 50%, about 30% to about 50%, or about 40% to about 50% BCL-2 target inhibition.

In some variations, the PI3K inhibitor, i.e., Compound C1, or a pharmaceutically acceptable salt thereof, is administered to the human at a dose not more than 150 mg, or less than 150 mg; or between 40 mg and 150 mg, between 50 mg and 150 mg, between 50 mg and 100 mg, or between 50 mg and 75 mg; or about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. In one embodiment, the method of the present application comprises administering Compound C1(S) or a pharmaceutically acceptable salt thereof at a dose of 50 mg, 100 mg, 150 mg, or 200 mg daily.

In some variations, the BCL-2 inhibitor, such as Compound B1, Compound B2 or Compound B3, or a pharmaceutically acceptable salt thereof, is administered to the human at a dose between 20 to 600 mg, between 20 to 400 mg, between 20 to 200 mg, between 100 to 400 mg, between 100 to 200 mg, about 20 mg, about 50 mg, about 100 mg, about 200 mg, about 300 mg, about 400 mg, about 500 mg, about 600 mg, about 700 mg, or about 800 mg. In some embodiments, the BCL-2 inhibitor is administered once weekly, once daily, once every other day, or once every five days. In some embodiments, the BCL-2 inhibitor is administered once daily for a period of one to seven days, followed by administration once weekly, once every other day or once every day for five days for the duration of treatment. In certain embodiments, the BCL-2 inhibitor is administered once daily for a period of one to seven days, followed by administration once weekly for the duration of treatment. In one embodiment, the method of the present application comprises administering Compound B1 or a pharmaceutically acceptable salt thereof at a dose of 50 mg, 100 mg, 200 mg, 300 mg, 400 mg, or 420 mg daily.

The therapeutically effective amount of the PI3K and BCL-2 inhibitors may be provided in a single dose or multiple doses to achieve the desired treatment endpoint. As used herein, “dose” refers to the total amount of an active ingredient to be taken each time by a human. The dose administered, for example for oral administration described above, may be administered once weekly, once daily (QD), twice daily (BID), three times daily, four times daily, or more than four times daily. In some embodiments, the PI3K and/or the BCL-2 inhibitors may be administered once daily. In some embodiments, the PI3K and/or the BCL-2 inhibitors may be administered twice daily. In some embodiments, the BCL-2 inhibitors may be administered once weekly or with a frequency that can vary between daily, every other day, once every 5 days, daily for 1, 2, 3, 4, 5, 6 or 7 days and then weekly or with a regimen that can combine these different frequencies and doses to result in a final dose and regimen that is tolerated and efficacious.

In one variation, the PI3K inhibitor, i.e., Compound C1, or a pharmaceutically acceptable salt thereof, is administered to the human at a dose of 50 mg twice daily. In another variation, the PI3K inhibitor, i.e., Compound C1, or a pharmaceutically acceptable salt thereof, is administered to human at a dose of 100 mg twice daily. In another variation, the PI3K inhibitor, i.e., Compound C1, or a pharmaceutically acceptable salt thereof, is administered to human at a dose of 150 mg twice daily. In another variation, the PI3K inhibitor, i.e., Compound C1, or a pharmaceutically acceptable salt thereof, is administered to human at a dose of 50-150 mg twice daily.

In one variation, the BCL-2 inhibitor, i.e., Compound B1, B2, or B3, is administered at a dose of about 50 mg to about 400 mg once daily. In one variation, the BCL-2 inhibitor, i.e., Compound B1, B2, or B3, is administered at a dose of about 50 mg once daily. In one variation, the BCL-2 inhibitor, i.e., Compound B1, B2, or B3, is administered at a dose of about 400 mg one daily.

In some variations, a therapeutically effective amount or a pharmaceutically effective amount refers to an amount that is sufficient to effect treatment, as defined below, when administered to a subject (e.g., a human) in need of such treatment. The therapeutically effective amount will vary depending upon the subject and disease condition being treated, the weight and age of the subject, the severity of the disease condition, the manner of administration and the like, which can readily be determined by one of ordinary skill in the art. For example, in one variation, a therapeutically effective amount of Compound A1, or a pharmaceutically acceptable salt or hydrate thereof, is an amount sufficient to modulate Btk expression, and thereby treat a human suffering an indication, or to ameliorate or alleviate the existing symptoms of the indication. In one variation, a therapeutically effective amount of Compound D1, or a pharmaceutically acceptable salt thereof, is an amount sufficient to modulate activity of anti-apoptotic Syk proteins, and thereby treat a human suffering an indication, or to ameliorate or alleviate the existing symptoms of the indication.

In another variation, the therapeutically effective amount of the Btk inhibitor, such as Compound A1, or a pharmaceutically acceptable salt or hydrate thereof, may be an amount sufficient to decrease a symptom of a disease or condition responsive to inhibition of Btk activity. In another variation, the therapeutically effective amount of the Syk inhibitor, such as Compound D1, or a pharmaceutically acceptable salt thereof, may be an amount sufficient to decrease activity of anti-apoptotic Syk proteins.

The therapeutically effective amount of the Btk and Syk inhibitors may also be determined based on data obtained from assays known in the art, including for example, the apoptosis assay described in Example 1 below. In one variation, the therapeutically effective amount of the Btk inhibitor in a human is a dose of from about 1 mg to about 200 mg. In another embodiment the Btk in a human is administered at a dose of from about 10 mg to about 200 mg. In another embodiment the Btk in a human is administered at a dose of from about 20 mg to about 160 mg. In other separate embodiments the Btk inhibitor is administered to a human at a dose of: a) from about 10 mg to about 100 mg, b) from about 50 mg to about 175 mg, c) from about 20 mg to about 150 mg, d) from about 75 mg to about 100 mg, and e) from about 100 mg to about 200 mg. Individual doses of the Btk inhibitor that may be administered to a human in need thereof include individual doses of 1 mg, 5 mg, 10 mg, 20 mg, 30 mg, 40 mg, 50 mg, 60 mg, 70 mg, 75 mg, 80 mg, 901 mg, 100 mg, 110 mg, 120 mg, 130 mg, 140 mg, 150 mg, 160 mg, 170 mg, 175 mg, and 200 mg. The doses of the Btk inhibitor may be administered as determined by a medical professional and may be administered once daily or may be delivered twice daily, three times daily, or four times daily.

In one variation, the therapeutically effective amount of the Syk inhibitor is a dose corresponding to 1 nmol to 200 nmol of the Syk inhibitor used in an apoptosis assay run with 10% serum.

In another variation, the Btk inhibitor, such as Compound A1, or a pharmaceutically acceptable salt or hydrate thereof, is administered to the human at a dose resulting in about 50%, about 55%, about 60%, about 65%, about 70%, about 75%, about 80%, about 90%, about 95%, or about 99% Btk target inhibition. In another variation, the Syk inhibitor, such as Compound B1, or a pharmaceutically acceptable salt thereof, is administered to the human at a dose resulting in about 50%, about 55%, about 60%, about 65%, about 70%, about 75%, about 80%, about 90%, about 95%, or about 99% Syk target inhibition.

In some variations, the Btk inhibitor, such as Compound A1, or a pharmaceutically acceptable salt or hydrate thereof, is administered to the human at a dose between 40 mg and 1200 mg, between 40 mg and 800 mg, between 40 mg and 600 mg, between 40 mg and 40 mg, about 100 mg, about 100 mg, about 200 mg, about 300 mg, about 400 mg, about 500 mg, about 600 mg, about 700 mg, or about 800 mg. In some variations, the Syk inhibitor, such as Compound B1, Compound B2 or Compound B3, or a pharmaceutically acceptable salt thereof, is administered to the human at a dose between 20 to 600 mg, between 20 to 400 mg, between 20 to 200 mg, about 20 mg, about 50 mg, about 100 mg, about 200 mg, about 300 mg, about 400 mg, about 500 mg, about 600 mg, about 700 mg, or about 800 mg.

In some embodiments, about 100 mg to 800 mg of the Syk inhibitor, Compound D1, or a pharmaceutically acceptable salt thereof, is administered to subject once or twice daily. In other embodiments, about 50 mg to 600 mg of Syk inhibitor, or a pharmaceutically acceptable salt thereof, is administered to the subject, once, twice, three times, or four times daily.

In one embodiment, about 100 mg of Compound D1, or a pharmaceutically acceptable salt thereof, is administered to subject once daily. In one embodiment, about 100 mg of Compound D1, or a pharmaceutically acceptable salt thereof, is administered to subject twice daily. In one embodiment, about 100 mg of Compound D1, or a pharmaceutically acceptable salt thereof, is administered to subject three times daily. In one embodiment, about 100 mg of Compound D1, or a pharmaceutically acceptable salt thereof, is administered to subject four times daily. In one embodiment, about 200 mg of Compound D1, or a pharmaceutically acceptable salt thereof, is administered to subject once daily. In one embodiment, about 200 mg of Compound D1, or a pharmaceutically acceptable salt thereof, is administered to subject twice daily. In one embodiment, about 200 mg of Compound D1, or a pharmaceutically acceptable salt thereof, is administered to subject three times daily. In one embodiment, about 200 mg of Compound D1, or a pharmaceutically acceptable salt thereof, is administered to subject four times daily. In one embodiment, about 300 mg of Compound D1, or a pharmaceutically acceptable salt thereof, is administered to subject once daily. In one embodiment, about 300 mg of Compound D1, or a pharmaceutically acceptable salt thereof, is administered to subject twice daily. In one embodiment, about 300 mg of Compound D1, or a pharmaceutically acceptable salt thereof, is administered to subject three times daily. In one embodiment, about 300 mg of Compound D1, or a pharmaceutically acceptable salt thereof, is administered to subject four times daily. In one embodiment, about 400 mg of Compound D1, or a pharmaceutically acceptable salt thereof, is administered to subject once daily. In one embodiment, about 400 mg of Compound D1, or a pharmaceutically acceptable salt thereof, is administered to subject twice daily. In one embodiment, about 400 mg of Compound D1, or a pharmaceutically acceptable salt thereof, is administered to subject three times daily. In one embodiment, about 400 mg of Compound D1, or a pharmaceutically acceptable salt thereof, is administered to subject four times daily.

Individual doses of Syk inhibitor Compound D1 that may be administered once, twice, three times, or four times daily to a human in need thereof also include 10 mg, 20 mg, 40 mg, 50 mg, 60 mg, 75 mg, 80, mg, 90 mg, 100 mg, 120 mg, 150 mg, 175 mg, 250 mg, 350 mg, 450 mg, 550 mg, 600 mg, 650 mg, 700 mg, 750 mg, and 800 mg. In one embodiment, the method of the present application comprises administering Compound D1 or pharmaceutically acceptable salt thereof at a dose of 200 mg, 400 mg, or 600 mg.

The therapeutically effective amount of Compound A1, Compound B1, Compound B2, Compound B3, Compound C1, Compound C1(S), Compound D1, and Formula II, or the pharmaceutically acceptable salt thereof, may be provided in a single dose or multiple doses to achieve the desired treatment endpoint. The below provides some illustrations that would be applicable to all compounds described herein. The therapeutically effective amount of the Btk and BCL-2 inhibitors may be provided in a single dose or multiple doses to achieve the desired treatment endpoint. As used herein, “dose” refers to the total amount of an active ingredient to be taken each time by a human. The dose administered, for example for oral administration described above, may be administered once weekly, once daily (QD), twice daily (BID), three times daily, four times daily, or more than four times daily. In some embodiments, the Btk and/or the BCL2 inhibitors may be administered once daily. In some embodiments, the Btk and/or the BCL-2 inhibitors may be administered twice daily. In some embodiments, the BCL2 inhibitors may be administered once weekly or with a frequency that can vary between daily, every other day, once every 5 days, daily for 1, 2, 3, 4, 5, 6 or 7 days and then weekly or with a regimen that can combine these different frequencies and doses to result in a final dose and regimen that is tolerated and efficacious. In one embodiment, the method of the present application comprises administering Compound A1 or a pharmaceutically acceptable hydrochloride salt thereof at a dose of 20 mg, 40 mg, 75 mg, 80 mg, 150 mg, or 200 mg daily; and Compound B1 or a pharmaceutically acceptable salt thereof at a dose of 200 mg, 300 mg, 400 mg, or 420 mg daily. In other embodiment, the method of the present application comprises administering Compound A1 or a pharmaceutically acceptable hydrochloride salt thereof at a dose of 20 mg, 40 mg, 80 mg, 150 mg, or 200 mg daily; and Compound C1(S) at a dose of 50 mg, 100 mg, 150 mg, or 200 mg daily. In additional embodiment, the method of the present application comprises administering Compound C1(S) at a dose of 50 mg, 100 mg, 150 mg, or 200 mg daily; and Compound B1 or a pharmaceutically acceptable salt thereof at a dose of 200 mg, 300 mg, 400 mg, or 420 mg daily. In one embodiment, the method of the present application comprises administering Compound A1 or a pharmaceutically acceptable hydrochloride salt thereof at a dose of 20 mg, 40 mg, 75 mg, 80 mg, 150 mg, or 200 mg daily; and Compound D1 or pharmaceutically acceptable mesylate salt thereof at a dose of 200 mg, 400 mg, or 600 mg daily. In one other embodiment, the method of the present application comprises administering Compound D1 or a pharmaceutically acceptable mesylate salt thereof at a dose of 200 mg, 400 mg, or 600 mg daily; and Compound B1 or a pharmaceutically acceptable salt thereof at a dose of 200 mg, 300 mg, 400 mg, or 420 mg daily.

In some embodiments, the method comprises administering to the subject in need thereof Compound A1 or a pharmaceutically acceptable hydrochloride salt thereof at a dose of 40 mg or 80 mg once daily; and Compound C1(S) at a dose of 100 mg once daily or 50 mg twice daily. In some other embodiments, the method comprises administering to the subject in need thereof Compound A1 or a pharmaceutically acceptable hydrochloride salt thereof at a dose of 40 mg or 80 mg once daily; Compound C1(S) at a dose of 100 mg once daily or 50 mg twice daily; and Compound B1 or pharmaceutically acceptable salt thereof at a dose of 200 mg, 400 mg, or 420 mg once daily. In some additional embodiments, the method comprises administering to the subject in need thereof Compound A1 or a pharmaceutically acceptable hydrochloride salt thereof at a dose of 40 mg or 80 mg once daily; Compound C1(S) at a dose of 100 mg once daily or 50 mg twice daily; and obinutuzumab at a dose of 1000 mg. In certain embodiments, the method comprises administering to the subject in need thereof Compound C1(S) at a dose of 50 mg twice daily or 100 mg once daily; and Compound B1 or a pharmaceutically acceptable salt thereof at a dose of 200 mg, 400 mg, or 420 mg once daily. In certain other embodiments, the method comprises administering to the subject in need thereof Compound C1(S) at a dose of 50 mg twice daily or 100 mg once daily; Compound B1 or a pharmaceutically acceptable salt thereof at a dose of 200 mg, 400 mg, or 420 mg once daily; and obinutuzumab at a dose of 1000 mg. In one embodiment, the method comprises administering to the subject in need thereof Compound A1 or a pharmaceutically acceptable hydrochloride salt thereof at a dose of 40 mg or 80 mg once daily; and Compound D1 or a pharmaceutically acceptable mesylate salt thereof at a dose of 400 mg once daily. In one other embodiment, the method comprises administering to the subject in need thereof Compound A1 or a pharmaceutically acceptable hydrochloride salt thereof at a dose of 40 mg or 80 mg once daily; Compound D1 or a pharmaceutically acceptable mesylate salt thereof at a dose of 400 mg once daily; and Compound B1 or a pharmaceutically acceptable salt thereof at a dose of 200 mg, 400 mg, or 420 mg once daily. In one embodiment, the method comprises administering to the subject in need thereof Compound A1 or a pharmaceutically acceptable hydrochloride salt thereof at a dose of 40 mg or 80 mg once daily; Compound D1 or a pharmaceutically acceptable mesylate salt thereof at a dose of 400 mg once daily; and obinutuzumab at a dose of 1000 mg. In other embodiment, the method comprises administering to the subject in need thereof Compound D1 or a pharmaceutically acceptable mesylate salt thereof at a dose of 400 mg once daily; and Compound B1 or a pharmaceutically acceptable salt thereof at a dose of 200 mg, 300 mg, 400 mg, or 420 mg daily. In some other embodiments, the method comprises administering to the subject in need thereof Compound D1 or a pharmaceutically acceptable mesylate salt thereof at a dose of 400 mg once daily; Compound B1 or a pharmaceutically acceptable salt thereof at a dose of 200 mg, 300 mg, 400 mg, or 420 mg daily; and obinutuzumab at a dose of 1000 mg. In additional embodiments, the method comprises administering to the subject in need thereof Compound A1 or a pharmaceutically acceptable hydrochloride salt thereof at a dose of 40 mg or 80 mg once daily; and Compound B1 or a pharmaceutically acceptable salt thereof at a dose of 200 mg, 300 mg, 400 mg, or 420 mg daily. In certain additional embodiments, the method comprises administering to the subject in need thereof Compound A1 or a pharmaceutically acceptable hydrochloride salt thereof at a dose of 40 mg or 80 mg once daily; Compound B1 or a pharmaceutically acceptable salt thereof at a dose of 200 mg, 300 mg, 400 mg, or 420 mg daily; and obinutuzumab at a dose of 1000 mg.

### Administration

The BTK inhibitor, such as Compound A1, and the BCL-2 inhibitors, such as Compound B1, Compound B2 and Compound B3, may be administered using any suitable methods known in the art. Additionally, Compound C1, Compound C1(S), Compound D1, Formula II, or pharmaceutically acceptable salt thereof, may be administered using any suitable methods known in the art. For example, the compounds may be administered bucally, ophthalmically, orally, osmotically, parenterally (intramuscularly, intraperitoneally intrasternally, intravenously, subcutaneously), rectally, topically, transdermally, or vaginally.

Further, in certain variations, the BTK inhibitor described herein may be administered prior, after or concurrently with the BCL-2 inhibitors described herein. In other variations, the PI3K-δ inhibitor described herein may be administered prior, after or concurrently with the BCL-2 inhibitors described herein. In other variations, the BTK inhibitor described herein may be administered prior, after or concurrently with the SYK inhibitors described herein. In some other variations, Compound A1, Compound B1, Compound C1(S), and/or Compound D1, or the pharmaceutically acceptable salt thereof may be administered prior, after or concurrently with each other. In certain other variations, Compound A1, Compound B1, Compound C1(S), and/or Compound D1, or the pharmaceutically acceptable salt thereof may be administered prior, after or concurrently with Obinutuzumab.

The PI3K inhibitor, i.e., Compound C1, and the BCL-2 inhibitors, such as Compound B1, Compound B2 and Compound B3, are administered using any suitable methods known in the art. For example, in certain variations, the compounds are administered bucally, ophthalmically, orally, osmotically, parenterally (intramuscularly, intraperitoneally intrasternally, intravenously, subcutaneously), rectally, topically, transdermally, or vaginally.

The Btk inhibitor, such as Compound A1, and the Syk inhibitors, such as Compound B1 may be administered using any suitable methods known in the art. For example, the compounds may be administered bucally, ophthalmically, orally, osmotically, parenterally (intramuscularly, intraperitoneally intrasternally, intravenously, subcutaneously), rectally, topically, transdermally, or vaginally.

In one variation, the PI3K inhibitor is administered orally. In one variation, the PI3K inhibitor is administered orally once daily or twice daily. In one variation, the BCL-2 inhibitor is administered orally. In one variation, the PI3K inhibitor and the BCL-2 inhibitor are each administered orally. Further, in certain variations, the PI3K inhibitor described herein are administered prior, after or concurrently with the BCL-2 inhibitors described herein. Further, in certain variations, the Btk inhibitor described herein may be administered prior, after or concurrently with the Syk inhibitors described herein.

In certain variations, the PI3K inhibitor is dosed prior to dosing with the BCL-2 inhibitor. For example, in a certain variation, the PI3K inhibitor is dosed at 50 mg to 150 mg twice daily for a specified period of time, followed by co-administration with the BCL-2 inhibitor. In certain variations, the PI3K inhibitor is dosed for a period of up to about 12 weeks prior to co-administration with the BCL-2 inhibitor. In certain variations, the PI3K inhibitor is dosed for a period of about 1 to 12 weeks, 4 to 12 weeks, 6 to 12 weeks, 8 to 12 weeks, 10 to 12 weeks, 2 weeks, 3 weeks, 4 weeks, 5 weeks, 6 weeks, 7 weeks, 8 weeks, 9 weeks, 10 weeks, 11 weeks or 12 weeks prior to co-administration with the BCL-2 inhibitor. In a certain variation, the PI3K inhibitor is dosed for a period of about 4 to 12 weeks or about 6 to 12 weeks prior to co-administration with the BCL-2 inhibitor. In certain variations, the PI3K inhibitor is dosed at 50 mg to 150 mg twice daily for a specified period of time, followed by co-administration with the BCL-2 inhibitor, wherein the BCL-2 inhibitor is administered at a dose from about 20 mg to about 400 mg.

In certain variations, the BCL-2 inhibitor is dosed prior to dosing with the PI3K inhibitor. For example, in a certain variation, the BCL-2 inhibitor is dosed at 50 mg to 150 mg twice daily for a specified period of time, followed by co-administration with the PI3K inhibitor. In certain variations, the BCL-2 inhibitor is dosed for a period of up to about 12 weeks prior to co-administration with the PI3K inhibitor. In certain variations, the BCL-2 inhibitor is dosed for a period of about 1 to 12 weeks, 4 to 12 weeks, 6 to 12 weeks, 8 to 12 weeks, 10 to 12 weeks, 2 weeks, 3 weeks, 4 weeks, 5 weeks, 6 weeks, 7 weeks, 8 weeks, 9 weeks, 10 weeks, 11 weeks or 12 weeks prior to co-administration with the PI3K inhibitor. In a certain variation, the BCL-2 inhibitor is dosed for a period of about 4 to 12 weeks or about 6 to 12 weeks prior to co-administration with the PI3K inhibitor. In certain variations, the BCL-2 inhibitor is dosed at about 20 mg to about 400 mg daily or weekly for a specified period of time, followed by co-administration with the PI3K inhibitor, wherein the PI3K inhibitor is dosed from 50 mg to 150 mg twice daily.

In certain variations, the PI3K inhibitor is dosed concomitantly with the BCL-2 inhibitor. For example, in a certain variation, the PI3K inhibitor is dosed at 50 mg to 150 mg twice daily and the BCL-2 inhibitor is dosed at 20 mg to 400 mg once daily or weekly.

In certain variations, the PI3K inhibitor dose increases over the course of treatment. In certain variations, the BCL-2 inhibitor dose increases over the course of treatment. For example, in certain variations, the BCL-2 inhibitor is dosed at an initial dose ranging from about 20 mg to about 50 mg and is increased over the period of treatment to a final dose ranging from about 200 mg to about 600 mg. Similarly, in certain variations, the PI3K inhibitor is dosed at an initial dose of about 50 mg and is increased over the period of treatment to a final dose ranging from about 100 mg to about 150 mg. In certain variations, the dose of the BCL-2 inhibitor is increased weekly. In certain variations, the dose of the PI3K inhibitor is increased weekly or monthly.

Compound A1 80 mg may beadministered orally once daily and Compound C1 50 mg may be administered orally twice daily. Dosing of both agents will begin on Week 1, Day 1 of the treatment and continue at approximately the same time each day until the completion of treatment which may last for 24 weeks, 48 weeks, 96 weeks or 104 weeks or longer. Compound A1 may be supplied as 10 mg (8×10 mg) capsules or as 20 mg (4×20 mg) tablets or 80 mg tablets. Compound C1 may be supplied as 50 mg tablets.

Obinutuzumab will be administered as intravenous infusions of 1000 mg each over treatment which may be 21 weeks. A test dose of 100 mg will be administered on Week 1 Day 1. If this dose is tolerated, the remainder of the full dose will be administered on Day 1. Alternatively, the remaining 900 mg will be administered on Day 2. Subsequent infusions will be administered on Week 2 Day 1, Week 3 Day 1, Week 5 Day 1, and then every 4 weeks through Week 21 or end of treatment. Additional anti-CD20 inhibitors suitable for the methods described herein include, but are not limited to, rituximab, ibritumomab tiuxetan, tositumomab, ofatumumab, ocaratuzumab, veltuzumab, and the like.

Compound A1 may be administered orally once or twice daily depending on cohort, beginning on Cycle 1, Day 1 of the treatment and thereafter at approximately the same time each day until end of treatment. In the Compound A1 and Compound C1 arm, Compound C1 may be administered orally twice daily, beginning on Cycle 1, Day 2 and at approximately the same time as Compound A1. In the Compound A1 and Compound D1 arm, Compound D1 may be administered orally as per assigned treatment group, beginning on Cycle 1, Day 2. Compound Almay be supplied as 10 mg and 25 mg capsules. Compound C1 may be supplied as 50 mg and 100 mg tablets. Compound D1 may be supplied as 200 mg tablets. As used herein the treatment cycle may be 7 days, 14 days, 28 days, 1 month, 2 months, 3 months, 4 months, 5 months, 6 months, 7 months, 8 months, or 9 months. The treatment cycle may be repeated one, two, three, four, five, six, seven, eight, nine, ten times; or may continue for six months, seven months, eight months, nine months, ten months, eleven months, twelve months, fourteen months, fifteen months, eighteen months, twenty months, twenty-four months or continuously. In one embodiment, the treatment cycle may be 28 days. In another embodiment, the treatment may continue for six months, nine months, or eighteen months.

### Pharmaceutical Compositions

The BTK and BCL-2 inhibitors may be administered in the form of pharmaceutical compositions. For example, in some variations, the BTK inhibitor described herein may be present in a pharmaceutical composition comprising the BTK inhibitor, and at least one pharmaceutically acceptable vehicle. In some variations, the BCL-2 inhibitors described herein may be present in a pharmaceutical composition comprising the BCL-2 inhibitor, and at least one pharmaceutically acceptable vehicle. Pharmaceutically acceptable vehicles may include pharmaceutically acceptable carriers, adjuvants and/or excipients, and other ingredients can be deemed pharmaceutically acceptable insofar as they are compatible with other ingredients of the formulation and not deleterious to the recipient thereof.

This disclosure therefore provides pharmaceutical compositions that contain the BTK and BCL-2 inhibitors as described herein, and one or more pharmaceutically acceptable vehicle, such as excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In certain variations, the PI3K and BCL-2 inhibitors are administered in the form of pharmaceutical compositions. For example, in some variations, the PI3K inhibitor described herein is present in a pharmaceutical composition comprising the BCL-2 inhibitor, and at least one pharmaceutically acceptable vehicle. In some variations, the BCL-2 inhibitors described herein are present in a pharmaceutical composition comprising the BCL-2 inhibitor, and at least one pharmaceutically acceptable vehicle. Pharmaceutically acceptable vehicles may include pharmaceutically acceptable carriers, adjuvants and/or excipients, and other ingredients can be deemed pharmaceutically acceptable insofar as they are compatible with other ingredients of the formulation and not deleterious to the recipient thereof.

This disclosure therefore provides pharmaceutical compositions that contain the PI3K and BCL-2 inhibitors as described herein, and one or more pharmaceutically acceptable vehicle, such as excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The Btk and Syk inhibitors may be administered in the form of pharmaceutical compositions. For example, in some variations, the Btk inhibitor described herein may be present in a pharmaceutical composition comprising the Btk inhibitor, and at least one pharmaceutically acceptable vehicle. In some variations, the Syk inhibitors described herein may be present in a pharmaceutical composition comprising the Syk inhibitor, and at least one pharmaceutically acceptable vehicle. Pharmaceutically acceptable vehicles may include pharmaceutically acceptable carriers, adjuvants and/or excipients, and other ingredients can be deemed pharmaceutically acceptable insofar as they are compatible with other ingredients of the formulation and not deleterious to the recipient thereof.

This disclosure therefore provides pharmaceutical compositions that contain the Btk and Syk inhibitors as described herein, and one or more pharmaceutically acceptable vehicle, such as excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In some embodiments, the pharmaceutical compositions described herein are formulated in a unit dosage form. The term “unit dosage forms” refers to physically discrete units suitable as unitary dosages for human subjects, each unit containing a predetermined quantity of active material calculated to produce the desired therapeutic effect, in association with a suitable pharmaceutical excipient. In some variations, the pharmaceutical compositions described herein are in the form of a tablet, capsule, or ampoule.

In certain embodiments, the BTK inhibitor described herein, such as Compound A1, or a pharmaceutically acceptable salt or hydrate thereof, is formulated as a tablet. In some variations, such tablet may comprise a mesylate salt of Compound D1, such as a mono-mesylate or a bis-mesylate salt thereof, or a hydrate thereof. Such tablet comprising Compound A1, for example, may be prepared by suitable methods known in the art, such as spray-drying and granulation (e.g., dry granulation).

In certain embodiments, the PI3K inhibitor described herein, such as Compound C1, or a pharmaceutically acceptable salt or hydrate thereof, is formulated as a tablet. In some variations, such tablet includes a hydrochloride salt of Compound C1, or a hydrate thereof. Such tablet comprising Compound C1, for example, may be prepared by suitable methods known in the art, such as spray-drying and granulation (e.g., dry granulation).

### Additional Therapeutic Agents

In the present disclosure, in some aspects, the combination described herein may be further used or combined with a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof.

Chemotherapeutic agents may be categorized by their mechanism of action into, for example, the following groups: anti-metabolites/anti-cancer agents, such as pyrimidine analogs (floxuridine, capecitabine, and cytarabine); purine analogs, folate antagonists and related inhibitors antiproliferative/antimitotic agents including natural products such as vinca alkaloid (vinblastine, vincristine) and microtubule such as taxane (paclitaxel, docetaxel), vinblastin, nocodazole, epothilones and navelbine, epidipodophyllotoxins (etoposide, teniposide); DNA damaging agents (actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, etoposide, triethylenethiophosphoramide); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (adriamycin), daunorubicin, dactinomycin, eniposide, epirubicin, etoposide, idarubicin, irinotecan and mitoxantrone, topotecan, irinotecan), corticosteroids (cortisone, dexamethasone, hydrocortisone, methylpednisolone, prednisone, and prednisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin.

As used herein the term “chemotherapeutic agent” or “chemotherapeutic” (or “chemotherapy,” in the case of treatment with a chemotherapeutic agent) is meant to encompass any non-proteinaceous (i.e, non-peptidic) chemical compound useful in the treatment of cancer. Examples of chemotherapeutic agents include alkylating agents such as thiotepa and cyclophosphamide (CYTOXAN®); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammall and calicheamicin phil1, see, e.g., Agnew, Chem. Intl. Ed. Engl, 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; PSK; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (TAXOL®, Bristol Meyers Squibb Oncology, Princeton, N.J.) and docetaxel (TAXOTERE®, Rhone-Poulenc Rorer, Antony, France); chlorambucil; gemcitabine (GEMZAR®); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; FOLFIRI (fluorouracil, leucovorin, and irinotecan) and pharmaceutically acceptable salts, acids or derivatives of any of the above.

Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including NOLVADEX™), raloxifene, droloxifene, 4-hydroxytamoxifen, trioxifene, keoxifene, LY117018, onapristone, and toremifene (FARESTON®); inhibitors of the enzyme aromatase, which regulates estrogen production in the adrenal glands, such as, for example, 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®); and anti-androgens such as flutamide, nilutamide, bicalutamide, leuprohde, and goserelin; and pharmaceutically acceptable salts, acids or derivatives of any of the above.

The anti-angiogenic agents include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloproternase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,I-3,4-dehydroproline, thiaproline, .alpha.-dipyridyl, beta-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpba-2-antiplasmin, bisantrene, lobenzarit disodium, n-2-carboxyphenyl-4-chloroanthronilic acid disodium or “CCA”, thalidomide; angiostatic steroid, cargboxynaminolmidazole; metalloproteinase inhibitors such as BB94. Other anti-angiogenesis agents include antibodies, preferably monoclonal antibodies against these angiogenic growth factors: beta-FGF, alpha-FGF, FGF-5, VEGF isoforms, VEGF-C, HGF/SF and Ang-1/Ang-2. See Ferrara N. and Alitalo, K. “Clinical application of angiogenic growth factors and their inhibitors” (1999) Nature Medicine 5:1359-1364.

The anti-fibrotic agents include, but are not limited to, the compounds such as beta-aminoproprionitrile (BAPN), as well as the compounds disclosed in U.S. Pat. No. 4,965,288 to Palfreyman, et al., issued Oct. 23, 1990, entitled “Inhibitors of lysyl oxidase,” relating to inhibitors of lysyl oxidase and their use in the treatment of diseases and conditions associated with the abnormal deposition of collagen; U.S. Pat. No. 4,997,854 to Kagan, et al., issued Mar. 5, 1991, entitled “Anti-fibrotic agents and methods for inhibiting the activity of lysyl oxidase in situ using adjacently positioned diamine analogue substrate,” relating to compounds which inhibit LOX for the treatment of various pathological fibrotic states, which are herein incorporated by reference. Further exemplary inhibitors are described in U.S. Pat. No. 4,943,593 to Palfreyman, et al., issued Jul. 24, 1990, entitled “Inhibitors of lysyl oxidase,” relating to compounds such as 2-isobutyl-3-fluoro-, chloro-, or bromo-allylamine; as well as, e.g., U.S. Pat. No. 5,021,456; U.S. Pat. No. 5,5059,714; U.S. Pat. No. 5,120,764; U.S. Pat. No. 5,182,297; U.S. Pat. No. 5,252,608 (relating to 2-(1-naphthyloxymemyl)-3-fluoroallylamine); and U.S. Patent Application No. 2004/0248871, which are herein incorporated by reference. Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone. Also, the anti-fibrotic agents are copper chelating agents, penetrating or not penetrating the cells. Exemplary compounds include indirect inhibitors such compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, sodium-4-mercaptobutanesulphinate trihydrate.

The immunotherapeutic agents include and are not limited to therapeutic antibodies suitable for treating patients; such as abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8. The exemplified therapeutic antibodies may be further labeled or combined with a radioisotope particle, such as indium In 111, yttrium Y 90, iodine 1-131.

In a certain embodiments, the additional therapeutic agent is a nitrogen mustard alkylating agent. Nonlimiting examples of nitrogen mustard alkylating agents include chlorambucil.

Some chemotherapy agents suitable for treating lymphoma or leukemia include aldesleukin, alvocidib, antineoplaston AS2-1, antineoplaston A10, anti-thymocyte globulin, amifostine trihydrate, aminocamptothecin, arsenic trioxide, beta alethine, BCL-2 family protein inhibitor ABT-263, ABT-199, ABT-737, BMS-345541, bortezomib (VELCADE®), bryostatin 1, busulfan, carboplatin, campath-1H, CC-5103, carmustine, caspofungin acetate, clofarabine, cisplatin, Cladribine (Leustarin), Chlorambucil (Leukeran), Curcumin, cyclosporine, Cyclophosphamide (Cyloxan, Endoxan, Endoxana, Cyclostin), cytarabine, denileukin diftitox, dexamethasone, DT PACE, docetaxel, dolastatin 10, Doxorubicin (ADRIAMYCIN®, Adriblastine), doxorubicin hydrochloride, enzastaurin, epoetin alfa, etoposide, Everolimus (RAD001), fenretinide, filgrastim, melphalan, mesna, Flavopiridol, Fludarabine (Fludara), Geldanamycin (17-AAG), ifosfamide, irinotecan hydrochloride, ixabepilone, Lenalidomide (REVLIMID®, CC-5013), lymphokine-activated killer cells, melphalan, methotrexate, mitoxantrone hydrochloride, motexafin gadolinium, mycophenolate mofetil, nelarabine, oblimersen (Genasense) Obatoclax (GX15-070), oblimersen, octreotide acetate, omega-3 fatty acids, oxaliplatin, paclitaxel, PD0332991, PEGylated liposomal doxorubicin hydrochloride, pegfilgrastim, Pentstatin (Nipent), perifosine, Prednisolone, Prednisone, R-roscovitine (Selicilib, CYC202), recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, Styryl sulphones, tacrolimus, tanespimycin, Temsirolimus (CCl-779), Thalidomide, therapeutic allogeneic lymphocytes, thiotepa, tipifarnib, VELCADE® (bortezomib or PS-341), Vincristine (Oncovin), vincristine sulfate, vinorelbine ditartrate, Vorinostat (SAHA), vorinostat, and FR (fludarabine, rituximab), CHOP (cyclophosphamide, doxorubicin, vincristine, prednisone), CVP (cyclophosphamide, vincristine and prednisone), FCM (fludarabine, cyclophosphamide, mitoxantrone), FCR (fludarabine, cyclophosphamide, rituximab), hyperCVAD (hyperfractionated cyclophosphamide, vincristine, doxorubicin, dexamethasone, methotrexate, cytarabine), ICE (iphosphamide, carboplatin and etoposide), MCP (mitoxantrone, chlorambucil, and prednisolone), R-CHOP (rituximab plus CHOP), R-CVP (rituximab plus CVP), R-FCM (rituximab plus FCM), R-ICE (rituximab-ICE), and R-MCP (R-MCP).

In one embodiment, the compound or combination described herein may be used or combined with one or more additional therapeutic agents. The one or more therapeutic agents include, but are not limited to, an inhibitor of Abl, activated CDC kinase (ACK), adenosine A2B receptor (A2B), apoptosis signal-regulating kinase (ASK), Auroa kinase, Bruton's tyrosine kinase (BTK), BET-bromodomain (BRD) such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptors (EGFR), focal adhesion kinase (FAK), Flt-3, FYN, glycogen synthase kinase (GSK), HCK, histone deacetylase (HDAC), IKK such as IKK3c, isocitrate dehydrogenase (IDH) such as IDH1, Janus kinase (JAK), KDR, lymphocyte-specific protein tyrosine kinase (LCK), lysyl oxidase protein, lysyl oxidase-like protein (LOXL), LYN, matrix metalloprotease (MMP), MEK, mitogen-activated protein kinase (MAPK), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK) such as protein kinase A, B, and/or C, PYK, spleen tyrosine kinase (SYK), serine/threonine kinase TPL2, serine/threonine kinase STK, signal transduction and transcription (STAT), SRC, serine/threonine-protein kinase (TBK) such as TBK1, TIE, tyrosine kinase (TK), vascular endothelial growth factor receptor (VEGFR), YES, or any combination thereof.

### Lymphoma or Leukemia Combination Therapy

Some chemotherapy agents are suitable for treating lymphoma or leukemia. These agents include aldesleukin, alvocidib, antineoplaston AS2-1, antineoplaston A10, anti-thymocyte globulin, amifostine trihydrate, aminocamptothecin, arsenic trioxide, beta alethine, BCL-2 family protein inhibitor ABT-263, ABT-199, ABT-737, BMS-345541, bortezomib (VELCADE®), bryostatin 1, busulfan, carboplatin, campath-1H, CC-5103, carmustine, caspofungin acetate, clofarabine, cisplatin, cladribine, chlorambucil, curcumin, cyclosporine, cyclophosphamide, cytarabine, denileukin diftitox, dexamethasone, DT-PACE (dexamethasone, thalidomide, cisplatin, doxorubicin, cyclophosphamide, and etoposide), docetaxel, dolastatin 10, doxorubicin, doxorubicin hydrochloride, enzastaurin, epoetin alfa, etoposide, everolimus (RAD001), fenretinide, filgrastim, melphalan, mesna, flavopiridol, fludarabine, geldanamycin (17 AAG), ifosfamide, irinotecan hydrochloride, ixabepilone, lenalidomide (REVLIMID®, CC-5013), lymphokine-activated killer cells, melphalan, methotrexate, mitoxantrone hydrochloride, motexafin gadolinium, mycophenolate mofetil, nelarabine, oblimersen, obatoclax (GX15-070), oblimersen, octreotide acetate, omega-3 fatty acids, oxaliplatin, paclitaxel, PD0332991, PEGylated liposomal doxorubicin hydrochloride, pegfilgrastim, pentostatin, perifosine, prednisolone, prednisone, R-roscovitine (seliciclib, CYC202), recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, styryl sulphones, tacrolimus, tanespimycin, temsirolimus (CCl-779), thalidomide, therapeutic allogeneic lymphocytes, thiotepa, tipifarnib, bortezomib (VELCADE®, PS-341), vincristine, vincristine sulfate, vinorelbine ditartrate, SAHA (suberanilohydroxamic acid, or suberoyl, anilide, and hydroxamic acid), FR (fludarabine and rituximab), CHOP (cyclophosphamide, doxorubicin, vincristine, and prednisone), CVP (cyclophosphamide, vincristine, and prednisone), FCM (fludarabine, cyclophosphamide, and mitoxantrone), FCR (fludarabine, cyclophosphamide, and rituximab), hyperCVAD (hyperfractionated cyclophosphamide, vincristine, doxorubicin, dexamethasone, methotrexate, and cytarabine), ICE (iphosphamide, carboplatin, and etoposide), MCP (mitoxantrone, chlorambucil, and prednisolone), R-CHOP (rituximab and CHOP), R-CVP (rituximab and CVP), R-FCM (rituximab and FCM), R-ICE (rituximab and ICE), and R MCP (rituximab and MCP).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP, CVP, FCM, MCP, and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP, R-FCM, R-CVP, and R MCP.

Examples of radioimmunotherapy for NHL/B-cell cancers include yttrium-90 ibritumomab tiuxetan (ZEVALIN®) and iodine-131 tositumomab (BEXXAR®).

### Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of BCL-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are temsirolimus (TORISEL®, CCI-779) and temsirolimus in combination with RITUXAN®, VELCADE®, or other chemotherapeutic agents.

Other recent therapies for MCL have been disclosed. Such examples include flavopiridol, PD0332991, R-roscovitine (selicicilib, CYC202), styryl sulphones, obatoclax (GX15-070), TRAIL, Anti-TRAIL death receptors DR4 and DR5 antibodies, temsirolimus (TORISEL®, CCl-779), everolimus (RAD001), BMS-345541, curcumin, SAHA, thalidomide, lenalidomide (REVLIMID®, CC-5013), and geldanamycin (17 AAG).

### Waldenstrom's Macroglobulinemia Combination Therapy

Therapeutic agents used to treat Waldenstrom's Macroglobulinemia (WM) include perifosine, bortezomib (VELCADE®), rituximab, sildenafil citrate (VIAGRA®), CC-5103, thalidomide, epratuzumab (hLL2-anti-CD22 humanized antibody), simvastatin, enzastaurin, campath-1H, dexamethasone, DT-PACE, oblimersen, antineoplaston A10, antineoplaston AS2-1, alemtuzumab, beta alethine, cyclophosphamide, doxorubicin hydrochloride, prednisone, vincristine sulfate, fludarabine, filgrastim, melphalan, recombinant interferon alfa, carmustine, cisplatin, cyclophosphamide, cytarabine, etoposide, melphalan, dolastatin 10, indium-111 monoclonal antibody MN-14, yttrium-90 humanized epratuzumab, anti-thymocyte globulin, busulfan, cyclosporine, methotrexate, mycophenolate mofetil, therapeutic allogeneic lymphocytes, yttrium-90 ibritumomab tiuxetan, sirolimus, tacrolimus, carboplatin, thiotepa, paclitaxel, aldesleukin, docetaxel, ifosfamide, mesna, recombinant interleukin-11, recombinant interleukin-12, BCL-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab, iodine-131 tositumomab, motexafin gadolinium, arsenic trioxide, tipifarnib, autologous human tumor-derived HSPPC-96, veltuzumab, bryostatin 1, PEGylated liposomal doxorubicin hydrochloride, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-cell Lymphoma Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and R ICE.

### Chronic Lymphocytic Leukemia Combination Therapy

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

### Articles of Manufacture and Kits

Compositions (including, for example, formulations and unit dosages) comprising a BTK inhibitor, as described herein, and compositions comprising a BCL-2 inhibitor, as described herein, can be prepared and placed in an appropriate container, and labeled for treatment of an indicated condition. Accordingly, provided is also an article of manufacture, such as a container comprising a unit dosage form of a BTK inhibitor and a unit dosage form of a BCL-2 inhibitor, as described herein, and a label containing instructions for use of the compounds. In some embodiments, the article of manufacture is a container comprising (i) a unit dosage form of a BTK inhibitor, as described herein, and one or more pharmaceutically acceptable carriers, adjuvants or excipients; and (ii) a unit dosage form of a BCL-2 inhibitor, as described herein, and one or more pharmaceutically acceptable carriers, adjuvants or excipients. In one embodiment, the unit dosage form for both the BTK inhibitor and the BCL-2 inhibitor is a tablet.

Compositions (including, for example, formulations and unit dosages) comprising a PI3K inhibitor, as described herein, and compositions comprising a BCL-2 inhibitor, as described herein, can be prepared and placed in an appropriate container, and labeled for treatment of an indicated condition. Accordingly, provided is also an article of manufacture, such as a container comprising a unit dosage form of a PI3K inhibitor and a unit dosage form of a BCL-2 inhibitor, as described herein, and a label containing instructions for use of the compounds. In some embodiments, the article of manufacture is a container comprising (i) a unit dosage form of a PI3K inhibitor, as described herein, and one or more pharmaceutically acceptable carriers, adjuvants or excipients; and (ii) a unit dosage form of a BCL-2 inhibitor, as described herein, and one or more pharmaceutically acceptable carriers, adjuvants or excipients. In one embodiment, the unit dosage form for both the PI3K inhibitor and the BCL-2 inhibitor is a tablet.

Compositions (including, for example, formulations and unit dosages) comprising a Btk inhibitor, as described herein, and compositions comprising a Syk inhibitor, as described herein, can be prepared and placed in an appropriate container, and labeled for treatment of an indicated condition. Accordingly, provided is also an article of manufacture, such as a container comprising a unit dosage form of a Btk inhibitor and a unit dosage form of a Syk inhibitor, as described herein, and a label containing instructions for use of the compounds. In some embodiments, the article of manufacture is a container comprising (i) a unit dosage form of a Btk inhibitor, as described herein, and one or more pharmaceutically acceptable carriers, adjuvants or excipients; and (ii) a unit dosage form of a Syk inhibitor, as described herein, and one or more pharmaceutically acceptable carriers, adjuvants or excipients. In one embodiment, the unit dosage form for both the Btk inhibitor and the Syk inhibitor is a tablet.

Kits also are contemplated. For example, a kit can comprise unit dosage forms of a BTK inhibitor, as described herein, and compositions comprising a BCL-2 inhibitor, as described herein, and a package insert containing instructions for use of the composition in treatment of a medical condition. In some embodiments, the kits comprises (i) a unit dosage form of the BTK inhibitor, as described herein, and one or more pharmaceutically acceptable carriers, adjuvants or excipients; and (ii) a unit dosage form of a BCL-2 inhibitor, as described herein, and one or more pharmaceutically acceptable carriers, adjuvants or excipients. In one embodiment, the unit dosage form for both the BTK inhibitor and the BCL-2 inhibitor is a tablet.

In another example, a kit can comprise unit dosage forms of a PI3K inhibitor, as described herein, and compositions comprising a BCL-2 inhibitor, as described herein, and a package insert containing instructions for use of the composition in treatment of a medical condition. In some embodiments, the kits comprises (i) a unit dosage form of the PI3K inhibitor, as described herein, and one or more pharmaceutically acceptable carriers, adjuvants or excipients; and (ii) a unit dosage form of a BCL-2 inhibitor, as described herein, and one or more pharmaceutically acceptable carriers, adjuvants or excipients. In one embodiment, the unit dosage form for both the PI3K inhibitor and the BCL-2 inhibitor is a tablet.

Kits also are contemplated. For example, a kit can comprise unit dosage forms of a Btk inhibitor, as described herein, and compositions comprising a Syk inhibitor, as described herein, and a package insert containing instructions for use of the composition in treatment of a medical condition. In some embodiments, the kits comprises (i) a unit dosage form of the Btk inhibitor, as described herein, and one or more pharmaceutically acceptable carriers, adjuvants or excipients; and (ii) a unit dosage form of a Syk inhibitor, as described herein, and one or more pharmaceutically acceptable carriers, adjuvants or excipients. In one embodiment, the unit dosage form for both the Btk inhibitor and the Syk inhibitor is a tablet.

The instructions for use in the kit may be for treating a cancer, including, for example, a hematologic malignancy, as further described herein.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

### Example 1

The study was conducted to evaluate the potency of the BTK inhibitor, Compound A1, to induce apoptosis in primary chronic lymphocytic leukemia (CLL) cells in the absence and presence of stromal cell co-culture with αIgM/αIgG/αCD40 co-stimulation. The secondary objective was to determine if the combination of Compound A1 with the BCL-2 inhibitor, Compound B1, could enhance the apoptotic effect of the single agents in primary CLL cells.

### Materials and Methods

Samples of Compounds A1 and B1 were prepared as 10 mM stocks in dimethyl sulfoxide (DMSO). Before use, compounds were either thawed from 10 mM DMSO stocks frozen in 0.75 mL polypropylene tubes at −20° C., or aliquoted from 10 mM DMSO stocks stored at room temperature in glass storage vials.

The reagents used in these assays are listed in Table 1.

TABLE 1
Reagents

| Reagents | Supplier | Catalog No. |
|---|---|---|
| BD Vacutainer CPT tubes | Becton Dickinson | 362753 |
| RPMI-1640 base medium | Sigma | R8758 |
| IMDM base medium | Life Technologies | 12440 |
| Fetal Bovine Serum | Gemini | 100-106 |
| 1X PBS^{+/+} | Life Technologies | 14040 |
| 1X PBS^{-/-} | Life Technologies | 14190 |
| Sodium pyruvate | Sigma | S8636 |
| HEPES | Sigma | H0887 |
| GlutaMax | Life Technologies | 35050-061 |
| Penicillin-Streptomycin | Sigma | P0781 |
| β-mercaptoethanol | Life Technologies | 21985-023 |
| DMSO | Sigma | D2650 |
| αIgM/αIgG | Jackson ImmunoResearch | 109-006-127 |
| αCD40 | RD Systems | MAB6321 |
| APC-Annexin V | BD Biosciences | 550475 |
| PE-αCD5 | BD Biosciences | 555353 |
| BV421-αCD19 | BD Biosciences | 562440 |
| PE-IgG1κ isotype control | BD Biosciences | 555749 |
| BV421-IgG1κ isotype control | BD Biosciences | 562438 |
| Fixation Buffer | BD Biosciences | 554655 |
| 96 MICRO WELL ™ Plates | Nunc | N249662 |
| V-Bottom 300 μL/Well | | |
| Standard Lids For | Nunc | N264122 |
| 96 MICROWELL ™ Plates | | |
| Clear Sterile PS | | |
| 96-well Microplate | Falcon | F353077 |
| Tissue-Culture Treated | | |
| U-Bottom w/Lid PS | | |
| 96-well 2 mL deep well | Costar | 3961 |
| conical bottom plates | | |
| 96-well plates U-bottom | Costar | 353077 |
| 300 μL/Well | | |
| Foil Seals | ATCC | 60-2400 |

The HS-5 human stromal cell line was obtained from ATCC (American Tissue Type Collection). Cells were maintained in lymphocyte growth medium (LGM): RPMI-1640 supplemented with 10% FBS, 1% penicillin-streptomycin, 10 mM HEPES, 1 mM sodium pyruvate, 55 μM β-mercaptoethanol, and 2 mM GlutaMax. With informed consent, whole blood was obtained from CLL subjects.

Peripheral blood mononuclear cells (PBMCs) were prepared from human whole blood obtained from CLL patients by Ficoll-separation in CPT tubes according to manufacturer's directions (Becton Dickinson). Isolated PBMCs were cryopreserved in freezing medium (50% IMDM, 40% FBS, and 10% DMSO) and held in the gas phase of liquid nitrogen until use. One day prior to plating of primary CLL cells, wells of a U-bottom 96-well plate were coated with 3.0×10⁴ HS-5 cells and allowed to adhere overnight in a humidified 37° C. incubator supplemented with 5% CO₂. The % CD5+/CD19⁺ cells isolated were quantified via FACS staining.

One day prior to plating of primary CLL cells, wells of a U-bottom 96-well plate were coated with 3.0×10⁴ HS-5 cells and allowed to adhere overnight in a humidified 37° C. incubator supplemented with 5% CO₂.

Frozen primary CLL PBMCs were thawed, washed once in LGM, and rested for 3-5 hours in LGM at 37° C. prior to plating. Cells were then centrifuged at room temperature for 10 minutes and resuspended at 1.0-2.5×10⁵ cells/mL in LGM for plating. Assay wells were set up in U-bottom 96-well tissue culture plates in the absence or presence of HS-5 co-culture. Compounds were diluted as described below. Assay plates were incubated for 1 hour prior to stimulation with αIgM/αIgG (7.8 μg/well) and αCD40 (4 μg/well). Positive and negative controls were set up. Cells were incubated for 66-72 hours in a 37° C. CO₂ incubator and assayed for apoptosis.

Combination plates (Compound A1×Compound B1) were prepared as 8×3 or 3×3 matrices of compounds. In the 8×3 matrix, Compound A1 was prepared in half-log dilutions (10 μM-3 nM) and combined with Compound B1 (30, 10, 3 nM). Drug concentrations were selected based on free drug concentrations at clinically achievable exposures and adjusted for protein binding under the assay concentrations. Concentrations listed refer to final assay values.

Compounds were diluted from 10 mM DMSO stock solutions. Compound combinations were prepared by transferring 2 μl each of the appropriate working compound solutions or vehicle to wells of a 96-well polypropylene plate. A daughter plate was made by 100× dilution into media. The final assay plate was made by 10× dilution into the final assay plate described above. The final DMSO concentration was 0.1%.

### Flow Cytometry

Cells were transferred to a deep well (2 mL) assay block and rinsed with 1 mL cation free PBS (PBS^{-/-}). Cells were resuspended in Invitrogen's aqua Live/Dead reagent according to manufacturer's instructions and incubated 30 minutes on ice. Aqua Live/Dead was quenched with an equal volume of PBS^{+/+} and 4% FBS (FACS buffer). Cells were centrifuged and labeled with αCD5-PE, αCD19-BV421 and AnnexinV-APC in a total volume of 80 μL and incubated 30 minutes on ice. After labeling, cells were rinsed twice in FACS buffer and then fixed with BD Fixation buffer for 30 minutes on ice. Cells were rinsed twice with FACS buffer and analyzed.

The percentage of CD5⁺/CD19⁺ cells was quantified in the PBMCs to provide an assessment of the malignant cell population in each sample. To evaluate apoptosis, flow cytometric sampling of 50 μL cell suspensions (5,000-25,000 total events) were collected on a BD FACS Canto II instrument using a high throughput screen (HTS) autosampler. The CD5⁺CD19⁺ population was gated, and data were collected for the Annexin V⁻/Live-Dead⁻, AnnexinV⁺/Live-Dead⁻, AnnexinV⁺/Live-Dead⁺, and Annexin V⁻/Live-Dead⁺ CLL populations.

### Data Analysis

Annexin V⁺/Live-Dead⁻ or Annexin V⁻/Live-Dead⁺ cells were gated and percentages of positive cells in each population were recorded for each well and data were extracted to a flow cytometry standard (fcs) file. Average percentages of Annexin V⁺/Live-Dead⁻ and Annexin V⁻/Live-Dead⁺ were determined.

EC₅₀ values were calculated based on log concentration of drug and percent Annexin V⁺/Live-Dead⁻ and Annexin V⁻/Live-Dead⁺ cells using a 4 parameter nonlinear regression algorithm in Prism 6.01 of the GraphPad software (San Diego, Calif. USA, www.graphpad.com). Each run generated a single EC₅₀ based on duplicate well values at each compound dilution. EC₅₀ values and Hill slopes generated from each curve fit were reported.

Synergy of the drug combinations was assessed using the Bliss Model of Independence (Meletiadis J, et al., “Assessing in vitro combinations of antifungal drugs against yeasts and filamentous fungi: comparison of different drug interaction models.” Med Mycol 2005; 43(2):133-52). CLL cells were treated with the compounds both singly and in combination. The percentage of unaffected cells was determined for each individual compound at each concentration of the compound that was tested. The predicated additive interaction of the two individual compounds was calculated using the equation:

F_{A}=1−(F_{UA1}×F_{UA2}) 1)
where F_{A} is the Bliss predicted additive value for the combination, F_{UA1} is the percentage of cells unaffected by compound 1 alone, and F_{UA2} is the percentage of cells unaffected by compound 2 alone. The Bliss Score was calculated using the equation:

B=A−FA 2)
where B is the Bliss Score, A is the actual (observed) effect of the compound combination, and F_{A} is the Bliss predicted additive interaction calculated in equation (1). A Bliss Score equal to 0 indicates an additive interaction between the two compounds. A Bliss Score greater than 0 indicates a greater than additive or synergistic interaction between the two compounds. A Bliss Score less than 0 indicates a less than additive or antagonistic interaction between the two compounds. This process was repeated for each point in the 3×3 and 8×3 combination matrices used in these studies.

### Results

To determine whether Compound C1 could inhibit primary CLL cell apoptosis, a modified co-culture assay system was utilized. This in vitro system was designed to mimic the CLL cells' cellular interactions that exist with the stromal microenvironment found in vivo in lymph nodes or in the bone marrow. A summary of individual CLL subjects' PBMC apoptosis quantified in the presence or absence of αIgM/αIgG/αCD40 stimulation (+HS-5 co-culture) is presented in Table 2.

TABLE 2
Basal Apoptosis in CLL Subjects' Cells

| | | | CD5⁺/ | CLL Alone | | | CLL + HS5 | | |
|---|---|---|---|---|---|---|---|---|---|
| | | | CD19⁺ | AnnexinV⁺ | AnnexinV⁺ | | AnnexinV+ | AnnexinV+ | |
| Donor # | Sex | Age | Cells (%) | %^{a} | %^{b} | % Δ | %^{a} | %^{c} | % Δ |
| Donor 1^{d} | M | 69 | 86.9 | 12.1 | 10.3 | −1.8 | 12.3 | 16.3 | 4 |
| | | | | 11.6 | 11.0 | −0.6 | 11.0 | 17 | 6 |
| Donor 2 | M | 72 | 86.6 | 42.5 | 19.8 | −22.7 | 47.6 | 28.6 | −19 |
| Donor 3^{d} | F | 82 | 59.9 | 91.5 | 46.7 | −44.8 | 17.7 | 5.5 | −12.2 |
| | | | | 79.5 | 17.1 | −62.4 | 20.5 | 6.4 | −14.1 |
| Donor 4^{d} | F | 94 | 76.3 | 85.1 | 14.5 | −70.6 | 75.6 | 7.9 | −67.7 |
| | | | | 85.5 | 16.0 | −69.5 | 73.7 | 6.5 | −67.2 |
| Donor 5^{d} | M | 72 | 48.4 | 7.2 | 4.1 | −3.1 | 5.6 | 7.0 | 1.4 |
| | | | | 10.0 | 4.4 | −5.6 | nd | nd | nd |
| | | | | 6.6 | 5.4 | −1.2 | 8.7 | 7.8 | −0.9 |
| | | | | 11.0 | 4.0 | −7 | nd | nd | nd |
| | | | | 14.8 | 9.4 | −5.4 | 6.0 | 6.0 | 0 |
| Donor 6^{d} | F | 91 | 88.2 | 13.4 | 4.3 | −9.1 | nd | nd | nd |
| | | | | 14.6 | 4.5 | −10.1 | nd | nd | nd |
| Donor 7 | M | 69 | 87.3 | 5.2 | 8.0 | 2.8 | 6.6 | 8.0 | 1.4 |
| Donor 8^{d} | F | 57 | 59.9 | nd | nd | nd | 17.7 | 5.5 | −12.2 |
| | | | | 21.7 | 27.1 | 5.4 | 29.9 | 24.3 | −5.6 |
| Donor 9 | M | 76 | 72.5 | 18.0 | 15.4 | −2.6 | 9.9 | 11.2 | 1.3 |
| Donor 10^{d} | M | 57 | 90.5 | 17.0 | 5.7 | −11.3 | nd | nd | nd |
| | | | | 15.4 | 5.8 | −9.6 | nd | nd | nd |
| Donor 11^{d} | M | 79 | 29.0 | 96.5 | 45.1 | −51.4 | 85.9 | 29.3 | −56.6 |
| | | | | 95.7 | 40.0 | −55.7 | 80 | 22.7 | −57.3 |
| Donor 12 | M | 75 | 91.3 | 24.5 | 7.3 | −17.2 | 9.6 | 4.5 | −5.1 |
| Donor 13 | M | 70 | 84.0 | 47.9 | 31.4 | −16.5 | 16.4 | 12.5 | −3.9 |
| Donor 14^{d} | M | 78 | 65.1 | 24.1 | 10.3 | −13.8 | 7.4 | 6.9 | −0.5 |
| | | | | 52.2 | 17.6 | −34.6 | 10.4 | 9.8 | −0.6 |
| | | | | 52.9 | 20.0 | −32.9 | 13.8 | 15.1 | 1.3 |

^{a}no stimulation
^{b}αIgM/αIgG/αCD40 stimulation
^{c}αIgM/αIgG/αCD40 + HS-5 co-culture stimulation
^{d}data reported from multiple experiments due to high volume of available cells
nd not determined

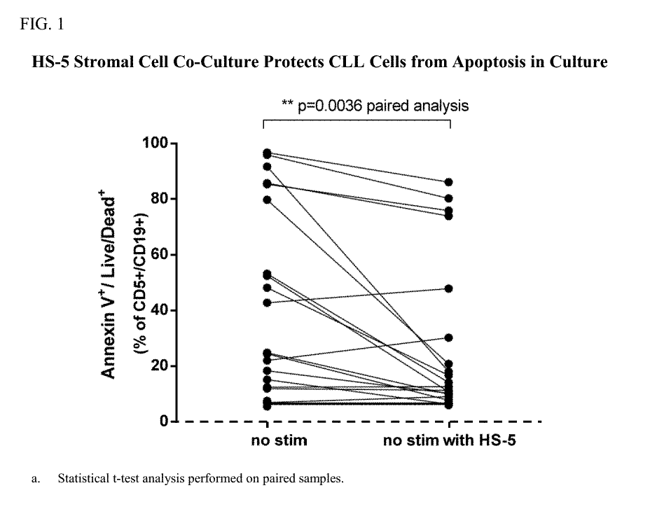

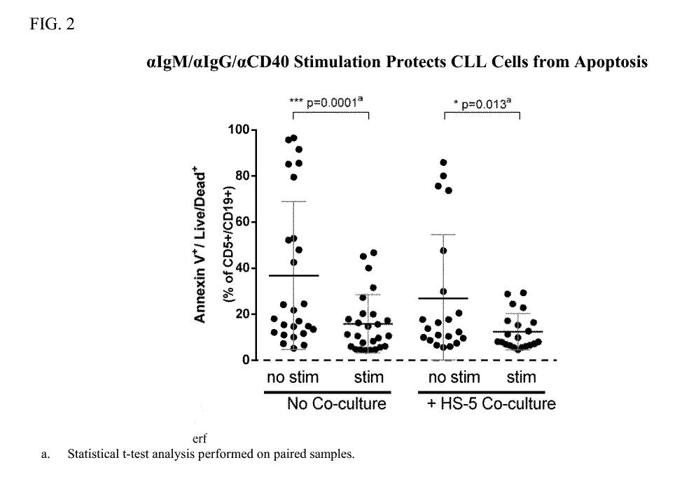

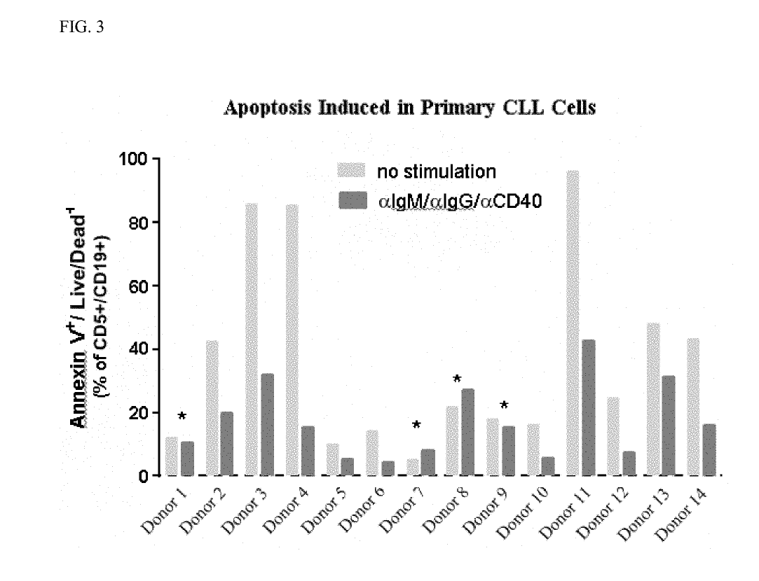

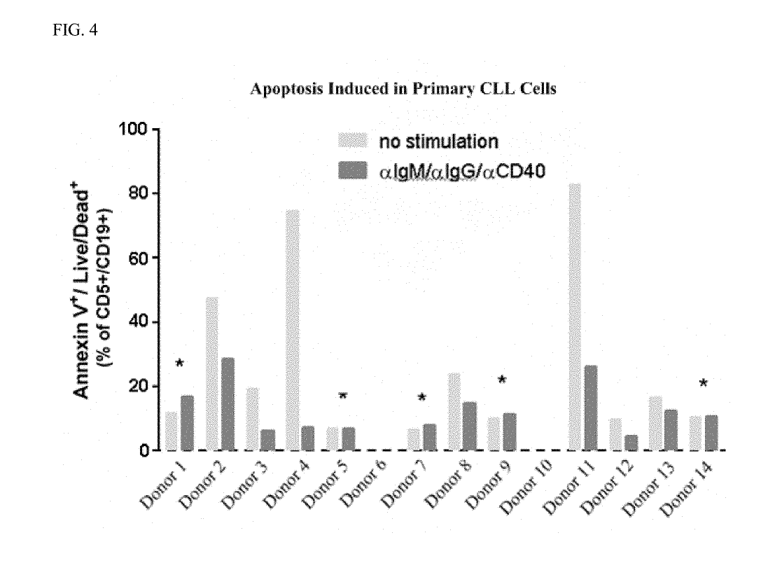

Using matched patients' CLL cells, there was a 37% increased cell viability when cells were cultured with HS-5 cells (27.4% apoptosis) as opposed to without HS-5 co-culture (43.8% apoptosis) (FIG. 1). B cell receptor (BCR) and CD40 receptor co-stimulation through the addition of αIgM/αIgG/αCD40 further protected the CLL cells from apoptosis (FIG. 2). There was a 57% increase in cell viability observed without αIgM/αIgG/αCD40 stimulation (36.8% apoptosis) versus with αIgM/αIgG/αCD40 stimulation (15.6% apoptosis) in the absence of HS-5 co-culture. The combination of αIgM/αIgG/αCD40 stimulation±HS-5 co-culture resulted in only 12.3% apoptosis versus 27.0% without stimulation±HS-5 co-culture, representing a 54% increase in viability. Although stimulation by αIgM/αIgG/αCD40 increased cell viability and protected CLL cells from apoptosis, 4 of 14 and 5 of 12 subjects' CLL cells in the absence or presence of HS-5 co-culture failed to show at least a 20% increased viability with stimulation, and may represent a less BCR-dependent subject population (FIGS. 3 and 4).

### Single Agent Induction of Apoptosis in Primary CLL Cells

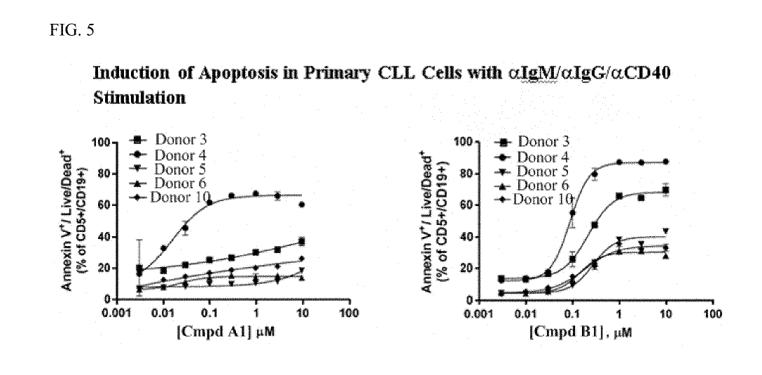

Compound A1 induced apoptosis in primary CLL cells from 5 donors stimulated with αIgM/αIgG/αCD40, resulting in a geometric mean EC50 (±standard deviation) value of 27.2±11.3 nM (N=4) and an average maximal apoptosis of 21.1% at 1 M (FIG. 3). In the presence of HS-5 co-culture, mean EC₅₀ values are not reported as apoptosis levels were generally not increased above background in 3/5 donors tested. The relatively low level of apoptosis observed with Compound A1 suggested that addition of the BCL-2 inhibitor, Compound B1, in combination with A1 might increase the overall apoptosis of the primary CLL cells with αIgM/αIgG/αCD40 co-stimulation. All compounds tested showed a dose-responsive effect in inducing apoptosis (FIG. 5). The magnitude of the response differed between donors and compounds. A comparison of the maximal apoptosis at 66-72 hr in 5 donors for Compound A1, is summarized in Table 3.

TABLE 3
Maximal Apoptosis Induced in Primary CLL Cells by Compound A1
Maximal % Apoptosis

| | HS-5 | +HS-5 |
|---|---|---|
| Donor No. | Compound A1 | Compound A1 |
| Donor 3 | 30.0 | 10.1 |
| Donor 4 | 67.3 | 45.5 |
| Donor 5 | 10.4 | nd |
| Donor 6 | 14.1 | nd |
| Donor 10 | 20.0 | nd |

a Values determined from duplicate wells with 1 μM compound, 66-72 hr post-compound addition
nd not determined

Combinations of Compound A1 with Compound B1 Increase Apoptosis in Primary CLL

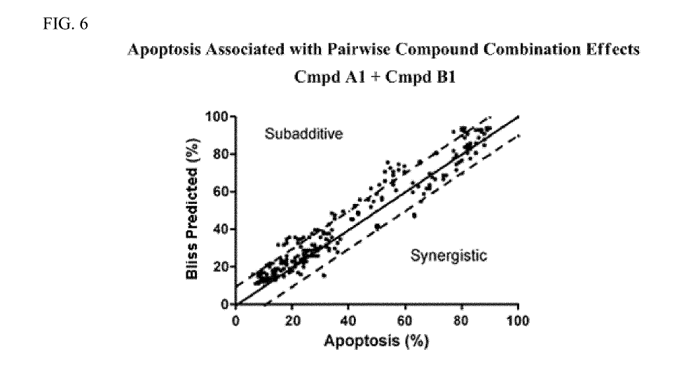

To assess for synergistic effects on apoptosis, the combination of Compound A1 with Compound B1 was tested in 8 individual primary CLL patient samples. To characterize the effects of the combinations, Compound A1 and Compound B1 were dosed in an 8×3 or 3×3 combination matrix as described above with CLL cells and treated for 66-72 h. The Bliss model of independence was used to score for synergy. The measured apoptosis versus the calculated Bliss score for synergy was plotted for each individual pairwise concentration assay point and the results are depicted in FIG. 6.

Addition of B1 in pairwise combination with A1 had in general an additive effect and some evidence of synergy. Combination of A1 with B1 resulted in generally additive to mildly synergistic responses (FIG. 6).

Protective or pro-survival signals can be induced in primary CLL cells by stimulation of the BCR or CD40 receptor and by signals induced by contact or cytokines produced by bone marrow stromal cells in the tumor microenvironment. In these studies, co-culture of CLL cells with stromal HS-5 cells, stimulation with BCR and CD40 receptor stimuli, or the combination of co-culture and BCR/CD40 receptor stimulation, led to an increase in CLL cell survival over the 66-72h assay period. The BTK inhibitor, Compound A1, induced apoptosis in primary CLL cells stimulated with αIgM/αIgG/αCD40 with a geometric mean EC₅₀ (±standard deviation) value of 27.2±11.3 nM (N=4) and an average maximal apoptosis of 21.1% screened at 1 μM compound.

Compound B1 has shown clinical efficacy in CLL. In these studies, all compounds were able to induce primary CLL cell apoptosis with αIgM/αIgG/αCD40 stimulation, +HS-5 cell co-culture. Addition of Compound B1 in pairwise combination with Compound A1 increased the apoptotic effect beyond that achievable with BTK inhibition alone. Of the 8 primary CLL samples tested with this combination, 6 showed a clear additive response. The combination of Compounds A1 and B1 resulted in additive to synergistic responses in all 8 primary CLL samples tested.

These results demonstrate that Compound A1 can cause apoptosis of primary CLL cells and can interfere with the protective or pro-survival signaling induced in primary CLL cells by BCR and CD40 receptor signaling and by signals induced by interactions between CLL and stromal cells. This apoptotic effect was enhanced by the addition of clinically achievable levels of Compound B1.

### Example 2

The study was conducted to evaluate the potency of the PI3K inhibitor, Compound C1, and the BCL-2 inhibitor, Compound B1, to induce apoptosis in primary chronic lymphocytic leukemia (CLL) cells with αIgM/αIgG/αCD40 co-stimulation in the absence and presence of stromal cell co-culture. The secondary objective was to determine if the combination of Compound C1 with the BCL-2 inhibitor, Compound B1, could enhance the apoptotic effect of the single agents in primary CLL cells with αIgM/αIgG/αCD40 co-stimulation in the absence and presence of stromal cell co-culture.

### Materials and Methods

Samples of Compounds C1 and B1 were prepared as 10 mM stocks in dimethyl sulfoxide (DMSO). Before use, compounds were either thawed from 10 mM DMSO stocks frozen in 0.75 mL polypropylene tubes at −20° C., or aliquoted from 10 mM DMSO stocks stored at room temperature in glass storage vials.

The reagents used in these assays are listed in Table 4.

TABLE 4
Reagents

| Reagents | Supplier | Catalog No. |
|---|---|---|
| BD Vacutainer CPT tubes | Becton Dickinson | 362753 |
| RPMI-1640 base medium | Sigma | R8758 |
| IMDM base medium | Life Technologies | 12440 |
| Fetal Bovine Serum | Gemini | 100-106 |
| 1X PBS^{+/+} | Life Technologies | 14040 |
| 1X PBS^{-/-} | Life Technologies | 14190 |
| Sodium pyruvate | Sigma | S8636 |
| HEPES | Sigma | H0887 |
| GlutaMax | Life Technologies | 35050-061 |
| Penicillin-Streptomycin | Sigma | P0781 |
| β-mercaptoethanol | Life Technologies | 21985-023 |
| DMSO | Sigma | D2650 |
| αIgM/αIgG | Jackson ImmunoResearch | 109-006-127 |
| αCD40 | RD Systems | MAB6321 |
| APC-Annexin V | BD Biosciences | 550475 |
| PE-αCD5 | BD Biosciences | 555353 |
| BV421-αCD19 | BD Biosciences | 562440 |
| PE-IgG1κ isotype control | BD Biosciences | 555749 |
| BV421-IgG1κ isotype control | BD Biosciences | 562438 |
| Fixation Buffer | BD Biosciences | 554655 |
| 96 MICROWELL ™ Plates | Nunc | N249662 |
| V-Bottom 300 μL/Well | | |
| Standard Lids For | Nunc | N264122 |
| 96 MICROWELL ™ Plates | | |
| Clear Sterile PS | | |
| 96-well Microplate | Falcon | F353077 |
| Tissue-Culture Treated | | |
| U-Bottom w/Lid PS | | |
| 96-well 2 mL deep well | Costar | 3961 |
| conical bottom plates | | |
| 96-well plates U-bottom | Costar | 353077 |
| 300 μL/Well | | |
| Foil Seals | ATCC | 60-2400 |

The HS-5 human stromal cell line was obtained from ATCC (American Tissue Type Collection). Cells were maintained in lymphocyte growth medium (LGM): RPMI-1640 supplemented with 10% FBS, 1% penicillin-streptomycin, 10 mM HEPES, 1 mM sodium pyruvate, 55 μM β-mercaptoethanol, and 2 mM GlutaMax. With informed consent, whole blood was obtained from CLL subjects (Bioreclamation, Westbury, N.Y.).

Peripheral blood mononuclear cells (PBMCs) were prepared from human whole blood obtained from CLL patients by Ficoll-separation in CPT tubes according to manufacturer's directions (Becton Dickinson). Isolated PBMCs were cryopreserved in freezing medium (50% IMDM, 40% FBS, and 10% DMSO) and held in the gas phase of liquid nitrogen until use. One day prior to plating of primary CLL cells, wells of a U-bottom 96-well plate were coated with 3.0×10⁴ HS-5 cells and allowed to adhere overnight in a humidified 37° C. incubator supplemented with 5% CO₂. The % CD5⁺/CD19⁺ cells isolated were quantified via FACS staining. One day prior to plating of primary CLL cells, wells of a U-bottom 96-well plate were coated with 3.0×10⁴ HS-5 cells and allowed to adhere overnight in a humidified 37° C. incubator supplemented with 5% CO₂.

Frozen primary CLL PBMCs were thawed, washed once in LGM, and rested for 3-5 hours in LGM at 37° C. prior to plating. Cells were then centrifuged at room temperature for 10 minutes and resuspended at 1.0-2.5×10⁵ cells/mL in LGM for plating. Assay wells were set up in U-bottom 96-well tissue culture plates in the absence or presence of HS-5 co-culture. Compounds were diluted as described below. Assay plates were incubated for 1 hour prior to stimulation with αIgM/αIgG (7.8 μg/well) and αCD40 (4 μg/well). Positive and negative controls were set up. Cells were incubated for 66-72 hours in a 37° C. CO₂ incubator and assayed for apoptosis.

Combination plates (Compound C1×Compound B1) were prepared as 4×9 or 4×3 matrices of compounds. In the 4×9 matrix, Compound C1 was used at concentrations of 0, 30, 100, or 300 nM and Compound B1 was used at concentrations of 0.8, 1.6, 3.13, 6.25, 12.5, 25, 50, 100, or 200 nM. In the 4×3 matrix, Compound C1 was used at concentrations of 0, 30, 120, or 480 nM and Compound B1 was used at concentrations of 3, 10, or 30 nM). Drug concentrations were selected based on free drug concentrations at clinically achievable exposures and adjusted for protein binding under the assay concentrations. Concentrations listed refer to final assay values.

Compounds were diluted from 10 mM DMSO stock solutions. Compound combinations were prepared by transferring 2 μl each of the appropriate working compound solutions or vehicle to wells of a 96-well polypropylene plate. A daughter plate was made by 100× dilution into media. The final assay plate was made by 10× dilution into the final assay plate described above. The final DMSO concentration was 0.1%.

### Flow Cytometry

Cells were transferred to a deep well (2 mL) assay block and rinsed with 1 mL cation free PBS (PBS^{-/-}). Cells were resuspended in Invitrogen's aqua Live/Dead reagent according to manufacturer's instructions and incubated 30 minutes on ice. Aqua Live/Dead was quenched with an equal volume of PBS^{+/+} and 4% FBS (FACS buffer). Cells were centrifuged and labeled with αCD5-PE, αCD19-BV421 and AnnexinV-APC in a total volume of 80 μL and incubated 30 minutes on ice. After labeling, cells were rinsed twice in FACS buffer and then fixed with BD Fixation buffer for 30 minutes on ice. Cells were rinsed twice with FACS buffer and analyzed.

The percentage of CD5⁺/CD19⁺ cells was quantified in the PBMCs to provide an assessment of the malignant cell population in each sample. To evaluate apoptosis, flow cytometric sampling of 50 μL cell suspensions (5,000-25,000 total events) were collected on a BD FACS Canto II instrument using a high throughput screen (HTS) autosampler. The CD5⁺CD19⁺ population was gated, and data were collected for the Annexin V⁻/Live-Dead⁻, AnnexinV⁺/Live-Dead⁻, AnnexinV⁺/Live-Dead⁺, and Annexin V⁻/Live-Dead⁺ CLL populations.

### Data Analysis

Annexin V⁺/Live-Dead⁻ or Annexin V⁻/Live-Dead⁺ cells were gated and percentages of positive cells in each population were recorded for each well and data were extracted to a flow cytometry standard (fcs) file. Average percentages of Annexin V⁺/Live-Dead⁻ and Annexin V⁻/Live-Dead⁺ were determined.

EC₅₀ values were calculated based on log concentration of drug and percent Annexin V⁺/Live-Dead⁻ and Annexin V⁻/Live-Dead⁺ cells using a four parameter nonlinear regression algorithm in Prism 6.01 of the GraphPad software (San Diego, Calif. USA, www.graphpad.com). Each run generated a single EC₅₀ based on duplicate well values at each compound dilution. EC₅₀ values and Hill slopes generated from each curve fit were reported.

Synergy of the drug combinations was assessed using the Bliss Model of Independence. (Meletiadis J, et al., “Assessing in vitro combinations of antifungal drugs against yeasts and filamentous fungi: comparison of different drug interaction models.” Med Mycol 2005; 43(2):133-52.) CLL cells were treated with the compounds both singly and in combination. The percentage of unaffected cells was determined for each individual compound at each concentration of the compound that was tested. The predicated additive interaction of the two individual compounds was calculated using the equation

F_{A}=1−(F_{UA1}×F_{UA2}) 1)
where F_{A} is the Bliss predicted additive value for the combination, F_{UA1} is the percentage of cells unaffected by compound 1 alone, and F_{UA2} is the percentage of cells unaffected by compound 2 alone. The Bliss Score was calculated using the equation:

B=A−F_{A} 2)
where B is the Bliss Score, A is the actual (observed) effect of the compound combination, and F_{A} is the Bliss predicted additive interaction calculated in equation (1). A Bliss Score equal to 0 indicates an additive interaction between the two compounds. A Bliss Score greater than 0 indicates a greater than additive or synergistic interaction between the two compounds. A Bliss Score less than 0 indicates a less than additive or antagonistic interaction between the two compounds. This process was repeated for each point in the 4×9 and 4×3 combination matrices used in these studies.

### Results

Stimulation of CLL cells by αIgM/αIgG/αCD40 led to a variable, but statistically significant, protection of primary CLL cells in vitro (p=0001), and co-culture with the stromal cell line, HS-5, in conjunction with αIgM/αIgG/αCD40 stimulation further reduced the observed level of apoptosis (p=0.0051). Compound C1 and Compound B1 were able to induce apoptosis in primary αIgM/αIgG/αCD40 stimulated CLL cells with an average maximal proportion of apoptotic cells of 23%, and 91.4%, respectively.

### Single Agent Induction of Apoptosis in Primary CLL Cells

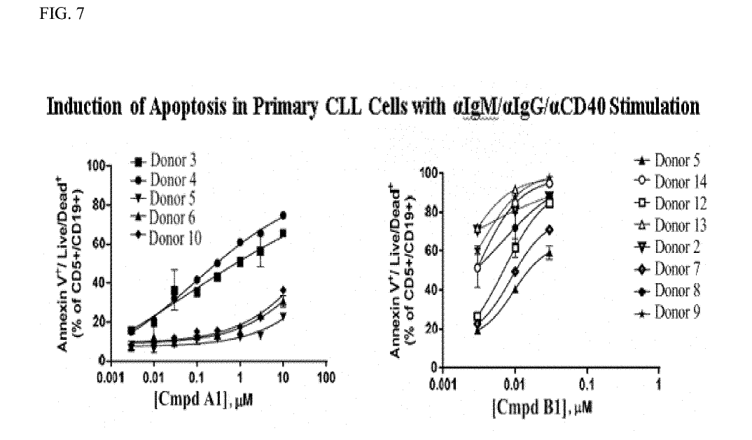

FIG. 7 demonstrates that Compound C1 showed a dose-responsive effect in inducing apoptosis. The magnitude of the response differed between donors. The maximal apoptosis at 66-72 hr in 5 donors for Compound C1 are summarized in Table 5. In contrast, Compound B1 was able to induce a greater level of apoptosis. At 30 nM, >60% apoptosis was observed in all CLL samples tested (FIG. 7).

TABLE 5
Maximal Apoptosis Induced in Primary CLL
Cells by Compound C1 Maximal % Apoptosis

Maximal % Apoptosis

| | HS-5 | +HS-5 |
|---|---|---|
| | Cmpd C1 | Cmpd C1 |
| Donor 3 | 50.8 | 15.2 |
| Donor 4 | 60.1 | 30.6 |
| Donor 5 | 12.1 | nd |
| Donor 6 | 15.0 | nd |
| Donor 10 | 17.6 | nd |

a Values determined from duplicate wells with 1 μM compound, 66-72 hr post-compound addition
nd not determined

Combinations of Compound C1 with Compound B1 Increase Apoptosis in Primary CLL

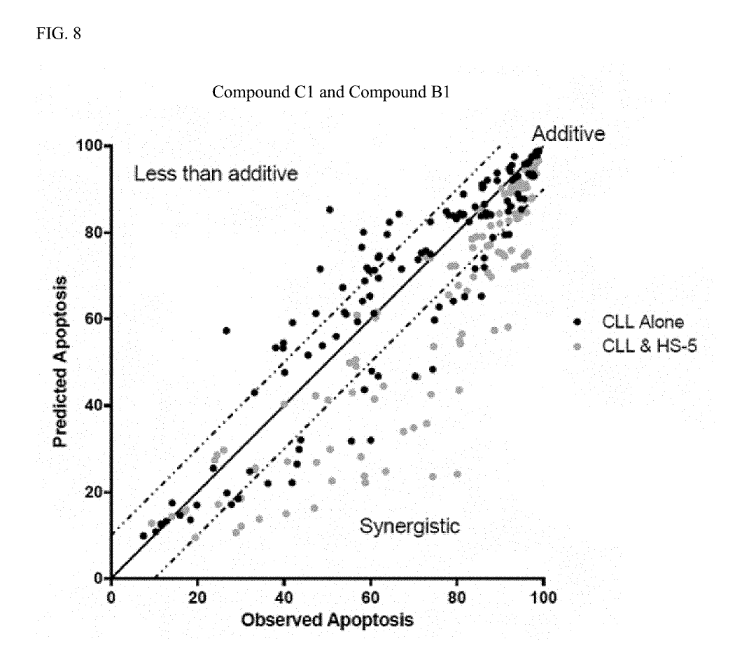

To assess for synergistic effects on apoptosis, the combination of Compound C1 with Compound B1 was tested in multiple individual primary CLL patient samples (i.e., 9 individual primary CLL patient samples). To characterize the effects of the combinations, Compound C1 and Compound B1 were dosed in a 4×9 or 4×3 combination matrix as described above with CLL cells and treated for 66-72 h. The Bliss model of independence was used to score for synergy. The measured apoptosis versus the calculated Bliss score for synergy was plotted for each individual pairwise concentration assay point and the results are depicted in FIG. 8.

The addition of Compound B1 to Compound C1 had an additive to synergistic effects on induction of apoptosis in primary CLL cells from all patients tested, and increased the maximal level of apoptosis (FIG. 8). In FIG. 8, each line represents data from primary CLL cells obtained from an individual patient donor. Nine different donors were utilized.

Protective or pro-survival signals can be induced in primary CLL cells by stimulation of the BCR or CD40 receptor and by signals induced by contact or cytokines produced by bone marrow stromal cells in the tumor microenvironment. In these studies, co-culture of CLL cells with stromal HS-5 cells, stimulation with BCR and CD40 receptor stimuli, or the combination of co-culture and BCR/CD40 receptor stimulation, led to an increase in CLL cell survival over the 66-72h assay period.

### Example 3

Samples of Compounds A1 and D1 were prepared as 10 mM stocks in dimethyl sulfoxide (DMSO). Before use, compounds were either thawed from 10 mM DMSO stocks frozen in 0.75 mL polypropylene tubes at −20° C., or aliquoted from 10 mM DMSO stocks stored at room temperature in glass storage vials.

The reagents used in these assays are listed in Table 6.

TABLE 6
Reagents

| Reagents | Supplier | Catalog No. |
|---|---|---|
| BD Vacutainer CPT tubes | Becton Dickinson | 362753 |
| RPMI-1640 base medium | Sigma | R8758 |
| IMDM base medium | Life Technologies | 12440 |
| Fetal Bovine Serum | Gemini | 100-106 |
| 1X PBS^{+/+} | Life Technologies | 14040 |
| 1X PBS^{-/-} | Life Technologies | 14190 |
| Sodium pyravate | Sigma | S8636 |
| HEPES | Sigma | H0887 |
| GlutaMax | Life Technologies | 35050-061 |
| Penicillin-Streptomycin | Sigma | P0781 |
| β-mercaptoethanol | Life Technologies | 21985-023 |
| DMSO | Sigma | D2650 |
| αIgM/αIgG | Jackson ImmunoResearch | 109-006-127 |
| αCD40 | RD Systems | MAB6321 |
| APC-Annexin V | BD Biosciences | 550475 |
| PE-αCD5 | BD Biosciences | 555353 |
| BV421-αCD19 | BD Biosciences | 562440 |
| PE-IgG1κ isotype control | BD Biosciences | 555749 |
| BV421-IgG1κ isotype control | BD Biosciences | 562438 |
| Fixation Buffer | BD Biosciences | 554655 |
| 96 Microwell ™ Plates | Nunc | N249662 |
| V-Bottom 300 μL/Well | | |
| Standard Lids For | Nunc | N264122 |
| 96 MicroWell ™ Plates | | |
| Clear Sterile PS | | |
| 96-well Microplate | Falcon | F353077 |
| Tissue-Culture Treated | | |
| U-Bottom w/Lid PS | | |
| 96 well 2 mL deep well | Costar | 3961 |
| conical bottom plates | | |
| 96-well plates U-bottom | Costar | 353077 |
| 300 μL/Well | | |
| Foil Seals | ATCC | 60-2400 |

The HS-5 human stromal cell line was obtained from ATCC (American Tissue Type Collection). Cells were maintained in lymphocyte growth medium (LGM): RPMI-1640 supplemented with 10% FBS, 1% penicillin-streptomycin, 10 mM HEPES, 1 mM sodium pyruvate, 55 μM β-mercaptoethanol, and 2 mM GlutaMax. With informed consent, whole blood was obtained from CLL subjects.

Peripheral blood mononuclear cells (PBMCs) were prepared from human whole blood obtained from CLL patients by Ficoll-separation in CPT tubes according to manufacturer's directions (Becton Dickinson). Isolated PBMCs were cryopreserved in freezing medium (50% IMDM, 40% FBS, and 10% DMSO) and held in the gas phase of liquid nitrogen until use. One day prior to plating of primary CLL cells, wells of a U-bottom 96-well plate were coated with 3.0×10⁴ HS-5 cells and allowed to adhere overnight in a humidified 37° C. incubator supplemented with 5% CO₂. The % CD5⁺/CD19⁺ cells isolated were quantified via FACS staining. One day prior to plating of primary CLL cells, wells of a U-bottom 96-well plate were coated with 3.0×104 HS-5 cells and allowed to adhere overnight in a humidified 37° C. incubator supplemented with 5% CO₂.

Frozen primary CLL PBMCs were thawed, washed once in LGM, and rested for 3-5 hours in LGM at 37° C. prior to plating. Cells were then centrifuged at room temperature for 10 minutes and resuspended at 1.0-2.5×10⁵ cells/mL in LGM for plating. Assay wells were set up in U-bottom 96-well tissue culture plates in the absence or presence of HS-5 co-culture. Compounds were diluted as described below. Assay plates were incubated for 1 hour prior to stimulation with αIgM/αIgG (7.8 μg/well) and αCD40 (4 μg/well). Positive and negative controls were set up. Cells were incubated for 66-72 hours in a 37° C. CO₂ incubator and assayed for apoptosis.

Combination plates (Compound A1×Compound D1) were prepared as 8×3 or 3×3 matrices of compounds. In the 8×3 matrix, Compound A1 was prepared in half-log dilutions (10 μM-3 nM) and combined with either 600, 300, and 100 nM or 100, 30, and 10 nM Compound B1. In the 3×3 matrix, A1 (100, 30, 10 nM) was combined with B1 (100, 30, 10 nM). Drug concentrations were selected based on free drug concentrations at clinically achievable exposures and adjusted for protein binding under the assay concentrations. Concentrations listed refer to final assay values.

Compounds were diluted from 10 mM DMSO stock solutions. Compound combinations were prepared by transferring 2 μl each of the appropriate working compound solutions or vehicle to wells of a 96-well polypropylene plate. A daughter plate was made by 100× dilution into media. The final assay plate was made by 10× dilution into the final assay plate described above. The final DMSO concentration was 0.1%.

Flow Cytometry

Cells were transferred to a deep well (2 mL) assay block and rinsed with 1 mL cation free PBS (PBS^{-/-}). Cells were resuspended in Invitrogen's aqua Live/Dead reagent according to manufacturer's instructions and incubated 30 minutes on ice. Aqua Live/Dead was quenched with an equal volume of PBS^{+/+} and 4% FBS (FACS buffer). Cells were centrifuged and labeled with αCD5-PE, αCD19-BV421 and AnnexinV-APC in a total volume of 80 μL and incubated 30 minutes on ice. After labeling, cells were rinsed twice in FACS buffer and then fixed with BD Fixation buffer for 30 minutes on ice. Cells were rinsed twice with FACS buffer and analyzed.

The percentage of CD5⁺/CD19⁺ cells was quantified in the PBMCs to provide an assessment of the malignant cell population in each sample. To evaluate apoptosis, flow cytometric sampling of 50 μL cell suspensions (5,000-25,000 total events) were collected on a BD FACS Canto II instrument using a high throughput screen (HTS) autosampler. The CD5⁺CD19⁺ population was gated, and data were collected for the Annexin V⁻/Live-Dead⁻, AnnexinV⁺/Live-Dead⁻, AnnexinV⁺/Live-Dead⁺, and Annexin V⁻/Live-Dead⁺ CLL populations.

Data Analysis

Annexin V⁺/Live-Dead⁻ or Annexin V⁻/Live-Dead⁺ cells were gated and percentages of positive cells in each population were recorded for each well and data were extracted to a flow cytometry standard (fcs) file. Average percentages of Annexin V⁺/Live-Dead⁻ and Annexin V⁻/Live-Dead⁺ were determined.

EC50 values were calculated based on log concentration of drug and percent Annexin V⁺/Live-Dead⁻ and Annexin V⁻/Live-Dead⁺ cells using a 4 parameter nonlinear regression algorithm in Prism 6.01 of the GraphPad software (San Diego, Calif. USA, www.graphpad.com). Each run generated a single EC50 based on duplicate well values at each compound dilution. EC50 values and Hill slopes generated from each curve fit were reported.

Synergy of the drug combinations was assessed using the Bliss Model of Independence. CLL cells were treated with the compounds both singly and in combination. The percentage of unaffected cells was determined for each individual compound at each concentration of the compound that was tested. The predicated additive interaction of the two individual compounds was calculated using the equation

F_{A}=1−(F_{UA1}×F_{UA2}) 1)
where F_{A} is the Bliss predicted additive value for the combination, F_{UA1} is the percentage of cells unaffected by compound 1 alone, and F_{UA2} is the percentage of cells unaffected by compound 2 alone. The Bliss Score was calculated using the equation:

B=A−F_{A} 2)
where B is the Bliss Score, A is the actual (observed) effect of the compound combination, and F_{A} is the Bliss predicted additive interaction calculated in equation (1). A Bliss Score equal to 0 indicates an additive interaction between the two compounds. A Bliss Score greater than 0 indicates a greater than additive or synergistic interaction between the two compounds. A Bliss Score less than 0 indicates a less than additive or antagonistic interaction between the two compounds. This process was repeated for each point in the 3×3 and 8×3 combination matrices used in these studies.

Results

To determine whether Compound A1 could inhibit primary CLL cell apoptosis, a modified co-culture assay system was utilized. This in vitro system was designed to mimic the CLL cells' cellular interactions that exist with the stromal microenvironment found in vivo in lymph nodes or in the bone marrow. A summary of individual CLL subjects' PBMC apoptosis quantified in the presence or absence of αIgM/αIgG/αCD40 stimulation (+HS-5 co-culture) is presented in

TABLE 7
Basal Apoptosis in CLL Subjects' Cells

| | | | CD5⁺/ | CLL Alone | | | CLL + HS5 | | |
|---|---|---|---|---|---|---|---|---|---|
| | | | CD19⁺ | AnnexinV⁺ | AnnexinV⁺ | | AnnexinV+ | AnnexinV+ | |
| Donor # | Sex | Age | Cells (%) | %^{a} | %^{b} | % Δ | %^{a} | %^{c} | % Δ |
| Donor 1^{d} | M | 69 | 86.9 | 12.1 | 10.3 | −1.8 | 12.3 | 16.3 | 4 |
| | | | | 11.6 | 11.0 | −0.6 | 11.0 | 17 | 6 |
| Donor 2 | M | 72 | 86.6 | 42.5 | 19.8 | −22.7 | 47.6 | 28.6 | −19 |
| Donor 3^{d} | F | 82 | 59.9 | 91.5 | 46.7 | −44.8 | 17.7 | 5.5 | −12.2 |
| | | | | 79.5 | 17.1 | −62.4 | 20.5 | 6.4 | −14.1 |
| Donor 4^{d} | F | 94 | 76.3 | 85.1 | 14.5 | −70.6 | 75.6 | 7.9 | −67.7 |
| | | | | 85.5 | 16.0 | −69.5 | 73.7 | 6.5 | −67.2 |
| Donor 5^{d} | M | 72 | 48.4 | 7.2 | 4.1 | −3.1 | 5.6 | 7.0 | 1.4 |
| | | | | 10.0 | 4.4 | −5.6 | nd | nd | nd |
| | | | | 6.6 | 5.4 | −1.2 | 8.7 | 7.8 | −0.9 |
| | | | | 11.0 | 4.0 | −7 | nd | nd | nd |
| | | | | 14.8 | 9.4 | −5.4 | 6.0 | 6.0 | 0 |
| Donor 6^{d} | F | 91 | 88.2 | 13.4 | 4.3 | −9.1 | nd | nd | nd |
| | | | | 14.6 | 4.5 | −10.1 | nd | nd | nd |
| Donor 7 | M | 69 | 87.3 | 5.2 | 8.0 | 2.8 | 6.6 | 8.0 | 1.4 |
| Donor 8^{d} | F | 57 | 59.9 | nd | nd | nd | 17.7 | 5.5 | −12.2 |
| | | | | 21.7 | 27.1 | 5.4 | 29.9 | 24.3 | −5.6 |
| Donor 9 | M | 76 | 72.5 | 18.0 | 15.4 | −2.6 | 9.9 | 11.2 | 1.3 |
| Donor 10^{d} | M | 57 | 90.5 | 17.0 | 5.7 | −11.3 | nd | nd | nd |
| | | | | 15.4 | 5.8 | −9.6 | nd | nd | nd |
| Donor 11^{d} | M | 79 | 29.0 | 96.5 | 45.1 | −51.4 | 85.9 | 29.3 | −56.6 |
| | | | | 95.7 | 40.0 | −55.7 | 80 | 22.7 | −57.3 |
| Donor 12 | M | 75 | 91.3 | 24.5 | 7.3 | −17.2 | 9.6 | 4.5 | −5.1 |
| Donor 13 | M | 70 | 84.0 | 47.9 | 31.4 | −16.5 | 16.4 | 12.5 | −3.9 |
| Donor 14^{d} | M | 78 | 65.1 | 24.1 | 10.3 | −13.8 | 7.4 | 6.9 | −0.5 |
| | | | | 52.2 | 17.6 | −34.6 | 10.4 | 9.8 | −0.6 |
| | | | | 52.9 | 20.0 | −32.9 | 13.8 | 15.1 | 1.3 |

^{a}no stimulation
^{b}αIgM/αIgG/αCD40 stimulation
^{c}αIgM/αIgG/αCD40 + HS-5 co-culture stimulation
^{d}data reported from multiple experiments due to high volume of available cells
nd not determined

Using matched patients' CLL cells, there was a 37% increased cell viability when cells were cultured with HS-5 cells (27.4% apoptosis) as opposed to without HS-5 co-culture (43.8% apoptosis) (FIG. 1. Importantly, B cell receptor (BCR) and CD40 receptor co-stimulation through the addition of αIgM/αIgG/αCD40 further protected the CLL cells from apoptosis

FIG. 2 There was a 57% increase in cell viability observed without αIgM/αIgG/αCD40 stimulation (36.8% apoptosis) versus with αIgM/αIgG/αCD40 stimulation (15.6% apoptosis) in the absence of HS-5 co-culture. The combination of αIgM/αIgG/αCD40 stimulation±HS-5 co-culture resulted in only 12.3% apoptosis versus 27.0% without stimulation+HS-5 co-culture, representing a 54% increase in viability. Interestingly, although stimulation by αIgM/αIgG/αCD40 increased cell viability and protected CLL cells from apoptosis, 4 of 14 and 5 of 12 subjects' CLL cells in the absence or presence of HS-5 co-culture failed to show at least a 20% increased viability with stimulation, and may represent a less BCR-dependent subject population (FIG. 3 and FIG. 4

Single Agent Induction of Apoptosis in Primary CLL Cells

Compound A1 induced apoptosis in primary CLL cells from 5 donors stimulated with

αIgM/αIgG/αCD40, resulting in a geometric mean EC50 (±standard deviation) value of 27.2±11.3 nM (N=4) and an average maximal apoptosis of 21.1% at 1 μM (FIG. 3). In the presence of HS-5 co-culture, mean EC50 values are not reported as apoptosis levels were generally not increased above background in 3/5 donors tested. The relatively low level of apoptosis observed with Compound A1 suggested that addition of the Syk inhibitor, Compound D1, in combination with A1 might increase the overall apoptosis of the primary CLL cells with αIgM/αIgG/αCD40 co-stimulation. All compounds tested showed a dose-responsive effect in inducing apoptosis FIG. 5. The magnitude of the response differed between donors and compounds. A comparison of the maximal apoptosis at 66-72 hr in 5 donors for Compound A1, is summarized in Table 8. Compound D1 was able to induce the greatest level of apoptosis. At 30 nM, >60% apoptosis was observed in all CLL samples tested.

TABLE 8
Maximal Apoptosis Induced in Primary CLL
Cells by Compound A1 Maximal % Apoptosis

| Maximal % Apoptosis | |
|---|---|
| HS-5 | +HS-5 |

| Donor # | Cmpd A1 | Cmpd B1 | Cmpd A1 | Cmpd B1 |
|---|---|---|---|---|
| Donor 3 | 30.0 | 66.1 | 10.1 | 30.8 |
| Donor 4 | 67.3 | 87.3 | 45.5 | 80.8 |
| Donor 5 | 10.4 | 38.3 | nd | nd |
| Donor 6 | 14.1 | 31.6 | nd | nd |
| Donor 10 | 20.0 | 35.8 | nd | nd |

a Values determined from duplicate wells with 1 μM compound, 66-72 hr post- compound addition
nd not determined

Combinations of Compound A1 with Compound D1 Increase Apoptosis in Primary CLL

To assess for synergistic effects on apoptosis with Compound A1 combined with Compound B1, was tested in 8 individual primary CLL patient samples. To characterize the effects of the combinations, Compound A1 or Compound D1, were dosed in an 8×3 or 3×3 combination matrix with CLL cells and treated for 66-72 h. The Bliss model of independence was used to score for synergy. The measured apoptosis versus the calculated Bliss score for synergy was plotted for each individual pairwise concentration assay point and the results are depicted in FIG. 6

Addition of D1 in pairwise combination with A1 had in general an additive effect and some evidence of synergy. Combination of A1 with B1 resulted in a generally additive to mildly synergistic response FIG. 6).

Protective or pro-survival signals can be induced in primary CLL cells by stimulation of the BCR or CD40 receptor and by signals induced by contact or cytokines produced by bone marrow stromal cells in the tumor microenvironment. In these studies, co-culture of CLL cells with stromal HS-5 cells, stimulation with BCR and CD40 receptor stimuli, or the combination of co-culture and BCR/CD40 receptor stimulation, led to an increase in CLL cell survival over the 66-72h assay period. The BTK inhibitor, Compound A1, induced apoptosis in primary CLL cells stimulated with αIgM/αIgG/αCD40 with a geometric mean EC50 (±standard deviation) value of 27.2±11.3 nM (N=4) and an average maximal apoptosis of 21.1% screened at 1 μM compound.

Compound D1 has shown clinical efficacy in CLL. In these studies, all compounds were able to induce primary CLL cell apoptosis with αIgM/αIgG/αCD40 stimulation, +HS-5 cell co-culture. Addition of Compound D1 in pairwise combination with Compound A1 increased the apoptotic effect beyond that achievable with BTK inhibition alone. Of the 8 primary CLL samples tested with this combination, 6 showed a clear additive response. The combination of Compounds A1 and D1 resulted in additive to synergistic responses in all 8 primary CLL samples tested.

These results demonstrate that Compound A1 can cause apoptosis of primary CLL cells and can interfere with the protective or pro-survival signaling induced in primary CLL cells by BCR and CD40 receptor signaling and by signals induced by interactions between CLL and stromal cells. This apoptotic effect was enhanced by the addition of clinically achievable levels of Compound D1.

### Example 4

In the following example Compound A1 refers to a Btk inhibitor and Compound C1 refers to a P13K delta inhibitor or idelalisib and Compound D1 refers to an Syk inhibitor or entospletinib.

Study I: Target Population in the study are adults with relapsed or refractory CLL. Duration of Treatment would include Obinutuzumab will be administered for up to 8 doses over 21 weeks to subjects randomized to treatment with Compound A1+b (Compound C1)+obinutuzumab (Arm B). Combination treatment with the oral agents (Compound A1 and Compound C1) may be continue for all subjects for up to 104 weeks\Efficacy will be assessed per modified IWCLL 2008 criteria {Hallek et al 2008}:Lymph node, spleen and liver measurements by physical examination, complete blood count, lymph node, spleen and liver measurements by CT or MRI, peripheral blood MRD assessment, and bone marrow assessment including standard histopathology and MRD assessment.

The safety of the combination of Compound A180 mg once daily and idelalisib 50 mg twice daily with and without obinutuzumab will be evaluated in this study for at least 28 days. Benefits from combination therapy include the potential to achieve higher rates of response and improve duration of response and the potential for reduced toxicity with increased efficacy by the use of lower individual study drug doses in combination.

Study II: Subjects will be enrolled in two cohorts. The doses for each cohort are as follows. Dose level one only has one cohort wherein the dosage includes 50 mg twice daily of Compound C1 and 20 mg once daily of Compound A1. Dose level two include two cohorts. One cohort receives 50 mg twice daily of Compound C1 and 40 mg once daily of Compound A1. The other cohort receives 50 mg twice daily of Compound C1 and 20 mg twice daily of Compound A1. Dose level three also includes two cohorts. One cohort receives 50 mg twice daily of Compound C1 and to 80 mg once daily of Compound A1. The other cohort receives 50 mg twice daily of Compound C1 and 40 mg twice daily of Compound A1. Dose level four includes two cohorts. One cohort receives 50 mg twice daily of Compound C1 and 150 mg once daily Compound A1. The other cohort receives includes 50 mg twice daily of Compound C1 and 75 mg twice daily of Compound A1. Dose level five only includes one cohort wherein the dosage of Compound A1 is to be determined and the dosage of Compound C1 is 100 mg twice daily.

Study III: Subjects will be enrolled in the study with the starting dose will be 40 mg once daily of Compound A1 and 200 mg once daily of Compound D1. If 1 DLT occurs within 28 days from Cycle 1, Day 1 in Cohort 1A of Combination II, this cohort will be expanded to enroll 3 additional subjects. If ≥2 DLTs occur in Cohort 1A of Combination II, (ie, ≥2 subjects experience DLTs), development of the combination of Compound A1 and entospletinib will discontinue. If no DLTs in 3 subjects or <2 DLTs in up to 6 subjects are observed, then the dose will be escalated to dose Level 2.

Dose Level 2 will consist of 2 cohorts: Cohort 2A with Compound A1 80 mg once daily and Compound D1 200 mg once daily and Cohort 2B with Compound A1 40 mg once daily and Compound D1 400 mg once daily. The first 3 subjects enrolled in Dose Level 2 will be assigned to Cohort 2A; the next 3 subjects will be assigned to Cohort 2B. Dose level three will consist of 2 cohorts. One cohort with 150 mg once daily of Compound A1 and 200 mg once daily of Compound D1. The other cohort 40 mg once daily of Compound A1 and 400 mg once daily of Compound D1. Dose level four will also consist of two cohorts. One cohort with 150 mg once daily of Compound A1 and 400 mg once daily of Compound D1. The other cohort 40 mg twice daily of Compound A1 and 200 mg twice daily of Compound D1.

All available safety, tolerability, and PK data will be reviewed prior to proceeding to the next cohort. The maximum dose to be tested will be 150 mg total daily dose of Compound A1 and 400 mg total daily dose of Compound D1, however, the dose escalation will be adaptive, with cohorts for reduced dosing, intermediate dosing, or different schedule (once daily vs twice daily) added based on emerging safety, PK, pharmacodynamics, and efficacy results.

Target Population: Adults with relapsed or refractory FL, marginal zone lymphoma (MZL), CLL, small lymphocytic lymphoma (SLL), MCL, Waldenstrom's macroglobulinemia (WM), or non-GCB DLBCL who have measurable disease per standard criteria and require therapy.

The maximum participation in treatment period for any subject may be 2 years. Subject suitable for the treatment may have the following criteria.

(i) Diagnosis of FL, MZL, SLL, CLL (meeting IWCLL Criteria 2008), MCL, WM, or non-GCB DLBCL as documented by medical records and with histology based on criteria established by the World Health Organization (WHO). a) FL Grades 1, 2, or 3a; b) SLL with absolute lymphocyte count of <5×10⁹/L at initial diagnosis; c) MZL (splenic, nodal, or extra-nodal); d) WM, measureable disease defined as serum monoclonal IgM>0.5 g/dL or meeting at least 1 of the recommendations from the Second International Workshop on Waldenstrom's Macroglobulinemia for requiring treatment)

(ii) Prior treatment for FL, MZL, SLL, MCL, WM, or non-GCB DLBCL with ≥2 or for CLL≥1 chemotherapy-based or immunotherapy-based regimen who are not transplant eligible and have had either documented disease progression or no response (stable disease) to the most recent treatment regimen.

(iii) For diseases other than WM, presence of radiographically measurable lymphadenopathy or extra-nodal lymphoid malignancy (defined as the presence of ≥1 lesion that measures ≥2.0 cm in the longest dimension [LD] and ≥1.0 cm in the longest perpendicular dimension [LPD] as assessed by computed tomography [CT] or magnetic resonance imaging [MRI]).

(iv) All acute toxic effects of any prior antitumor therapy resolved to Grade ≤1 before the start of study therapy (with the exception of alopecia [Grade 1 or 2 permitted], or bone marrow parameters [any of Grade 1 or 2 permitted]).

(v) Eastern Cooperative Oncology Group (ECOG) Performance Status (PS)≤2

(vi) Adequate organ function defined as follows: a) Hematologic: Platelets ≥50×109/L; Hemoglobin ≥8.0 g/dL; ANC ≥1.0×109/L (without platelet transfusion or any growth factors within previous 7 days of the hematologic laboratory values obtained at screening visit); b) Hepatic: Aspartate transaminase (AST)/Alanine transaminase (ALT)≤2.5× upper limit of normal (ULN) Total or conjugated bilirubin ≤1.5×ULN; c) Renal: Serum Creatinine ≤1.5×ULN or creatinine clearance (CrCl) ≥60 mL/min as calculated by the Cockcroft-Gault method

Screening will commence with obtaining the subject's signed informed consent and will occur up to 28 days prior to the first dosing of study drug on Cycle 1, Day 1. Baseline tumor assessment according to disease type will be measured and characterized prior to Cycle 1, Day 1 to assess the subject's disease status prior to beginning treatment.

Subjects who meet eligibility criteria will receive a single dose of Compound A1 on Cycle 1, Day 1 and then initiate Compound C1 or Compound D1 in combination with Compound A1 on Cycle 1, Day 2. The first cycle will consist of 28 days (1 day of single agent Compound A1 and 27 days of combination treatment), and each subsequent cycle will be 28 days of combination treatment. The assigned combination drug will remain consistent throughout the study. Safety and efficacy will be assessed, including assessment of tumor response, physical exam, vitals, ECG, collection of blood samples PK, pharmacodynamics, and biomarkers, and assessment of AEs. In addition, subjects will undergo a CT (or MRI) scan every 12 weeks, except DLBCL and CLL. Subjects with DLBCL will have an additional scan at week 6. Subjects with CLL will undergo scans at baseline, 24 weeks, and at the time of progression. A subject who does not show evidence of disease progression by clinical assessment or by CT (or MRI) may continue treatment until disease progression (clinical or radiographic)

PK samples will be collected on Cycle 1, Day 1 at pre-dose and 0.5, 1, 2, 3, 4, 6, 8, and 12 hours (optional) post-dose of Compound A1 and Cycle 1, Days 2 and 8 at pre-dose and 0.5, 1, 2, 3, 4, 6, 8, 12, and 24 hours post-dose of Compound A1 and idelalisib or entospletinib. The 12-hour post-dose PK samples are optional A sparse PK sample will also be collected anytime on the first day of Cycles 2 to 6.

Blood samples for pharmacodynamics will be collected on Cycle 1, Day 1 at pre-dose, 2, and 6 hours post-dose; on Cycle 1, Days 2 and 8 at pre-dose, 2, 6, and 24 hours post-dose; and at the end of treatment or disease progression. When study drug is administered BID, the 24 hour sample will be collected 24-hours post-dose relative to the morning dose. The collection of some or all of these samples may not be feasible at the site due to shipment logistics depending on their geographic location. In addition, sampling time points may be eliminated or modified based upon emerging data.

No potentially curative therapy exists for the patient population to be evaluated in this study. Dosing of each component in the dose escalation phase of this study starts at the lowest dose which may provide benefit to patients; the starting dose for Compound A1 is < 1/12th of the MTD in monotherapy study, the starting dose of idelalisib is ⅓rd of the approved dose, and starting dose of entospletinib is ¼th of the dose demonstrated to be tolerable in monotherapy study (no MTD identified).

Assessments for AEs and monitoring for laboratory abnormalities will be conducted on Days 1, 8, 15, and 22 of the first 28-day cycle.

### Example 5

Frozen peripheral blood samples from adult patients of Chronic Lymphoid Leukemia (CLL) were used for this study.

A small part of the thaw sample was stained with specific monoclonal antibodies (mAbs) in order to determine the number of pathological cells and the best combination of markers to identify pathological cells in each sample. Additionally, Annexin V was included in this combination to evaluate the initial viability of the sample. To analyze both viability and the best pair of antibodies to identify the target cell population, 20 μL of a combination of Annexin V, binding buffer (2.4 g HEPES, 8.19 g NaCl, 0.37 g C12Ca, H2O to 1 L), and the following mAbs was added to each well: CD19/CD20/CD10/CD5/CD23/CD45 for CLL samples

Native environment blood components for cell culture: In order to get the Native Environment for the cells to be cultured, the plasma and the RBC from the peripheral blood (PB) from donors or CLL samples were added. The plasma fractions were stored at −80° C. until use. RBC was kept at 4° C. for a maximum of 35 days after with the addition of the anticoagulant citrate phosphate dextrose adenine solution (CPDA-1; Terumo Corporation, Tokyo, Japan) to this fraction (150 μl CPDA/ml RBCs). For supplementing the CLL cell culture media, we used a 1:1 proportion of these two fractions (plasma and RBC)

Proliferation CFSE staining: The Vybrant® CFDA SE Cell Tracer Kit (Invitrogen, Thermo Fisher Scientific, Waltham, Mass., USA) was used to measure cell proliferation. CLL cells were adjusted at 10×106 cells/ml in AIM-V AlbuMAX culture media without FBS. CFSE was added to a lml cell suspension at a final concentration of 5 aM. After addition of CFSE, cells were vortexed and incubated at room temperature for 10 min with continuous shaking and light protected. At the end of the incubation period, the cells were resuspended in cold culture media with 10% FBS (complete culture media) and kept on ice for 5 min following two washes in cold complete culture media and maintained at 4° C. until use.

Assay Preparation: Cryopreserved CLL samples was diluted with AIM-V AlbuMAX (Invitrogen) supplemented with plasma and RBC provided from different CLL progressive samples, 10% of Human Serum (Sigma), 2% HEPES, 1% Zell Shield antibiotic (Labclinics, Barcelona, Spain), 1% L-glutamine 200 mM (Lonza, Hopkinton, Mass.), 1 μg/ml CpG ODN and 50 ng/ml IL-2. This mixture was dispensed into 96-well plates containing the HS5 (100:1) cell line and transferred into a new 96-well plate containing differrent drugs. (HS-5 cell lines were seed in 96-well plates and incubated for 24 hours to allow cells to adhere). Drug plates were previously prepared using an Echo 550 Liquid Handler (LabCyte, Sunnyvale, Calif.) at different concentration points. The plates were incubated for 96 hours at 37_C in humidified air containing 5% CO₂. Later, proliferation and viability was tested by flow cytometry.

Viability staining: To lyse red blood cells, 180 mL of ammonium chloride lysis solution was added to each well (2 g KHCO3, 16.58 g NH4Cl, 0.074 g Na2-ethylenediaminetetraacetic acid [EDTA]_2H2O, H2O to 1 L). Following a 10 minute incubation period at 4° C., each plate was centrifuged for 5 minutes at 1200 rpm and the supernatant removed. The lysis step was performed twice. For staining, 20 μL of a combination of annexin-V and two best markers for each CLL sample, resuspended in binding buffer were added. After 15 minutes of incubation at room temperature in the dark, a wash step was performed using BB solution. The pellet was resuspended in 80 μL BB for analysis in Vivia's ExviTech platform.

ExviTech™ Platform: This novel flow cytometry based system incorporates a CyAn ADP cytometer (Beckman Coulter, Brea, Calif., US) and Vivia's proprietary novel End Point Sampler (EPS) plate handler. The EPS aspirates the contents of each well of the assay plate, and delivers contents to the flow cell of the cytometer. Each 96-well assay plate is collected as a single .fcs file from the CyAn cytometer. The EPS is run from the same computer as the cytometer, recording a second file for each well within the plate. This later file is a timing file, which is integrated with the .fcs file for data analysis by our proprietary software program, FCS Analyzer. This program is designed to separate the data from the 96 wells acquired by the cytometer as a single file, into 96 separate data sets based on the precise time in which each sample aliquot (each well) is aspired into the cytometer, and assigns well numbers to each data set. Each 96-well plate is then analyzed as a single file, but with the ability to examine each well individually as needed, corresponding to each different drug/concentration assayed.

Data Analysis: Response effect of each compound was measured by counting the number of live tumor cells that remains after the exposure to increasing concentrations of drug. Survival index percentage was calculated as the difference in the number of live cells in a well with drug vs basal level of cells in control wells without drug. Once the pathological cell subset was identified, we used Annexin V to exclude dying cells and measured only the number of live cells, in the drug wells and in the control wells. Those cells without Annexin V staining and with appropriate FSC/SSC were considered as live cells. FCS Analyzer was used to determine the effect of each of the individual drugs.

Single drug potency and efficacy was estimated by a modelling approach to dose-response experiments. The model used (Equation 1) to fit the data was the most common single-site sigmoidal dose-response inhibitory model based on the Hill equation, where the dependent variable (DV) analysed was the number of live pathological cells counted by the cytometer at every tested concentration of drug. Data points were fitted using the Levenburg Marquardt algorithm. A normalization of the results was followed, referring each data point to the basal level parameter (EO). This approach allows the calculation of the normalized area under the curve (AUC) value by integrating the curve function from the model between the lowest and highest concentrations.

$$Y = {E}_{0} \times [ \frac{{E}_{max} - {E}_{0}}{1 + {( \frac{{EC}_{50}}{X} )}^{2}} ] \qquad [ Equation 1 ]$$

Drug interaction analysis was carried out by calculating the Combination index as is described by Chou T. [2]. This is the easiest and most common way of measuring drugs interaction is the determination of the Combination Index, (Ci).

This index is calculated as the sum of the ratios of drug concentrations required to reach a particular effect (x) in the combination and the concentration required to reach the same effect when each drug is tested alone (Equation 3).

Ciₓ=(C_{A|B}/C_{A})+(C_{B|A}/C_{B}) [Equation 3]:

Where C_{A|B} and C_{B|A} are the concentrations of drug A and drug B respectively in the combination experiments, and C_{A} and C_{B} are the concentrations of each drug required to get the same effect when are tested as single drugs. These concentrations are calculated by interpolate the effect observed in combination, in each single drug curve.

The meaning of the Ci is indicated as follows:

Ciₓ<1: Synergism; Ciₓ˜1: Additive effect; Ciₓ>1: Antagonism

Summary Interactions: Survival Percentage is calculated as the difference in the number of live cells in a well with a combination of drugs vs basal level of cells in control wells without drugs. The following is a list of the results. The cell count for the following remains constant at 25. For ABT-199 and P13K-delta inhibitor in NP live tumor cells (PM_GL2_006), the average is 47.8, and the standard deviation is 11.8. For the same combination in (PM_GL2_007), the average is 68.4, and the standard deviation is 36.0. For the same combination in PM_GL2_012, the average is 82.3, and the standard deviation is 26.6. For the same combination of inhibitor using PR Live tumor cells, in PM_GL2_006, the average is 66.5, and the standard deviation is 17.2. For the same combination in (PM_GL2_007), the average is 72.5, and the standard deviation is 20.3. For the same combination in PM_GL2_012, the average is 125.1, and the standard deviation is 66.2.

For ABT-199 and Btk inhibitor in NP live tumor cells (PM_GL2_006), the average is 63.6 and the standard deviation is 13.1. For the same combination in (PM_GL2_007), the average is 59.3, and the standard deviation is 44.7. For the same combination in PM_GL2_012, the average is 103.6, and the standard deviation is 28.7. For the same combination of inhibitor using PR Live tumor cells, in PM_GL2_006, the average is 88.51, and the standard deviation is 20.6. For the same combination in (PM_GL2_007), the average is 63.2, and the standard deviation is 39.4. For the same combination in PM_GL2_012, the average is 100.6, and the standard deviation is 30.1.

For ABT-199 and Syk inhibitor in NP live tumor cells (PM_GL2_006), the average is 57.6, and the standard deviation is 18.3. For the same combination in (PM_GL2_007) the average is 35.3, and the standard deviation is 22.96. For the same combination in PM_GL2_012, the average is 102.0, and the standard deviation is 67.0. For the same combination of inhibitor using PR Live tumor cells, in PM_GL2_006, the average is 65.2, and the standard deviation is 20.8. For the same combination in (PM_GL2_007), the average is 42.2, and the standard deviation is 38.4. For the same combination in PM_GL2_012, average is 129.9, and the standard deviation is 81.8.

For PI3k and Btk inhibitors in NP live tumor cells (PM_GL2_006), the average is 73.2, and the standard deviation is 37.7. For the same combination in (PM_GL2_007) the average is 67.1, and the standard deviation is 40.7. For the same combination in PM_GL2_012, the average is 138.9, and the standard deviation is 59.2. For the same combination of inhibitor using PR Live tumor cells, in PM_GL2_006, the average is 70.9, and the standard deviation is 33.2. For the same combination in (PM_GL2_007), the average is 63.6, and the standard deviation is 36.4. For the same combination in PM_GL2_012, the average is 158.5, and the standard deviation is 83.6.

For Syk and Btk inhibitors in NP live tumor cells (PM_GL2_006), the average is 55.3, and the standard deviation is 15.3. For the same combination in (PM_GL2_007), the average is 32.3, and the standard deviation is 18.98. For the same combination in PM_GL2_012, the average is 76.6, and the standard deviation is 58.2. For the same combination of inhibitor using PR Live tumor cells, in PM_GL2_006, the average is 59.4, and the standard deviation is 21.1. For the same combination in (PM_GL2_007), the average is 30.1, and the standard deviation is 23.95. For the same combination in PM_GL2_012, the average is 98.4, and the standard deviation is 85.8.

For ABT-199 and PI3K-delta inhibitor in NP live tumor cells (PM_GL2_006) the cell count is 25, the average is 1.12, the and the standard deviation is 2.7. For the same combination in (PM_GL2_007) the cell count is 20 the average is 5413.3, and the standard deviation is 24122.3. For the same combination in PM_GL2_012 the cell count is 18, the average is 0.071, and the standard deviation is 0.083. For the same combination of inhibitor using PR Live tumor cells, in PM_GL2_006 the cell count is 24, the average is 0.18, and the standard deviation is 0.54. For the same combination in (PM_GL2_007) the cell count is 23, the average is 0.04, and the standard deviation is 0.046. For the same combination in PM_GL2_012 the cell count is 10, the average is 0.053 and the standard deviation is 0.047.

For ABT-199 and Btk inhibitor in NP live tumor cells (PM_GL2_006) The cell count is 24, the average is 4.7, and the standard deviation is 5.7. For the same combination in (PM_GL2_007) the cell count is 19, the average is 9482.5, and the standard deviation is 41331.7. For the same combination in PM_GL2_012 the cell count is 11, the average is 0.214, and the standard deviation is 0.321. For the same combination of inhibitor using PR Live tumor cells, in PM_GL2_006 the cell count is 19, the average is 0.569, and the standard deviation is 1.132. For the same combination in (PM_GL2_007) the cell count is 22, the average is 0.03, and the standard deviation is 0.046. For the same combination in PM_GL2 012 the cell count is 10, the average is 0.073 and the standard deviation is 0.078.

For ABT-199 and Syk inhibitor in NP live tumor cells (PM_GL2_006) the cell count is 24, the average is 47.605, and the standard deviation is 112.9. For the same combination in PM_GL2_007 the cell count is 25, the average is 11.5 and the standard deviation is 37.9. For the same combination in PM_GL2_012 the cell count is 17, the average is 0.614, and the standard deviation is 0.867. For the same combination of inhibitor using PR Live tumor cells, in PM_GL2_006 the cell count is 22, the average is 0.55 and the standard deviation is 0.488. For the same combination in (PM_GL2_007) the cell count is 22, the average is 0.078 and the standard deviation is 0.09. For the same combination in PM_GL2 012 the cell count is 10, average is 0.614, and the standard deviation is 0.867.

For PI3k and Btk inhibitors in NP live tumor cells (PM_GL2_006) there is no data. For the same combination in (PM_GL2_007) the cell count is 19, the average is 1.107 E+14, and the standard deviation is 4.43E+14. For the same combination in PM_GL2 012 there is no data. For the same combination of inhibitor using PR Live tumor cells, in PM_GL2_006 the cell count is 20, the average is 80.7, and the standard deviation is 359.3. For the same combination in (PM_GL2_007) the cell count is 22, the average is 543.5, and the standard deviation is 2527.695. For the same combination in PM_GL2_012 the cell count is 7, the average is 3.3, and the standard deviation is 6.8.

For Syk and Btk inhibitors in NP live tumor cells (PM_GL2_006) the cell count is 25, the average is 9.516 E+16, and the standard deviation is 4.758E+17. For the same combination in (PM_GL2_007) the cell count is 25, the average is 1029.1, and the standard deviation is 4447.058. For the same combination in PM_GL2_012 the cell count is 19, the average is 0.001, and the standard deviation is 0.002. For the same combination of inhibitor using PR Live tumor cells, in PM_GL2_006 the cell count is 25, the average is 6.911, and the standard deviation is 21.2. For the same combination in (PM_GL2_007) the cell count is 25, the average is 20.4, and the standard deviation is 95.7. For the same combination in PM_GL2 012 the cell count is 15, the average is 0.025 and the standard deviation is 0.033.

1. A method for treating cancer in a human in need thereof, comprising administering to the human a therapeutically effective amount of a BTK inhibitor which is 6-amino-9-[(3R)-1-(2-butynoyl)-3-pyrrolidinyl]-7-(4-phenoxyphenyl)-7,9-dihydro-8H-purin-8-one, or a pharmaceutically acceptable salt or hydrate thereof; and a therapeutically effective amount of one or more additional therapeutic agents, wherein the additional therapeutic agents are:
(a) a BCL-2 inhibitor selected from the group of:
(4-(4-{[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide),
4-(4-((4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide, and
4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide,
or a pharmaceutically acceptable salt or hydrate thereof;
(b) a Syk inhibitor;
(c) idelalisib and obinutuzumab;
(d) a P13K inhibitor or a pharmaceutically acceptable salt or hydrate thereof, and obinutuzumab;
(e) idelalisib and ABT-199;
(f) obinutuzumab and a Syk inhibitor;
(g) ABT-199 and a Syk inhibitor;
(h) obinutuzumab and ABT-199; or
(i) idelalisib;
wherein the Syk inhibitor is of the formula: 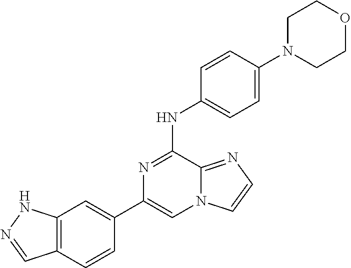 [SMILES: c1cn2cc(-c3ccc4cn[nH]c4c3)nc(Nc3ccc(N4CCOCC4)cc3)c2n1]
or a pharmaceutically acceptable salt or hydrate thereof, or of the formula: 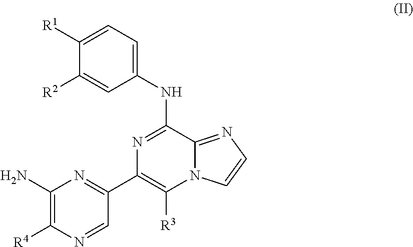
or a pharmaceutically acceptable salt or hydrate thereof, wherein
R¹ is selected from the group consisting of 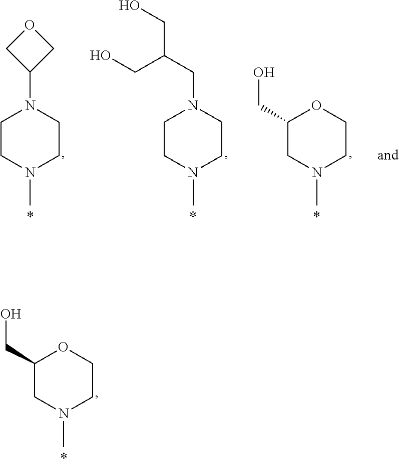
indicates the carbon atom of the indicated phenyl ring to which R¹ is attached,
R² is H or 2-hydroxyethoxyl,
R³ is H or methyl, and
R⁴ is H or methyl.

2. The method of claim 1, wherein the additional therapeutic agent is a BCL-2 inhibitor and in which the human is (i) refractory to at least one anti-cancer therapy, or (ii) is in relapse after treatment with at least one anti-cancer therapy, or both (i) and (ii).

3. The method of claim 1, wherein the additional therapeutic agent is
a Syk inhibitor of the formula: 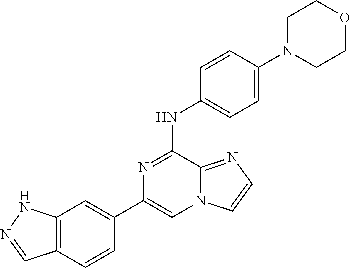 [SMILES: c1cn2cc(-c3ccc4cn[nH]c4c3)nc(Nc3ccc(N4CCOCC4)cc3)c2n1]
or a pharmaceutically acceptable salt or hydrate thereof, or of the formula: 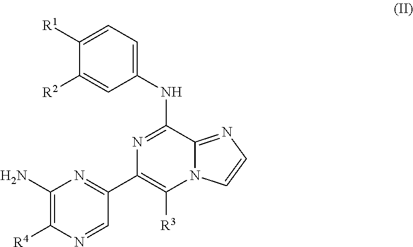
or a pharmaceutically acceptable salt or hydrate thereof, wherein
R¹ is selected from the group consisting of 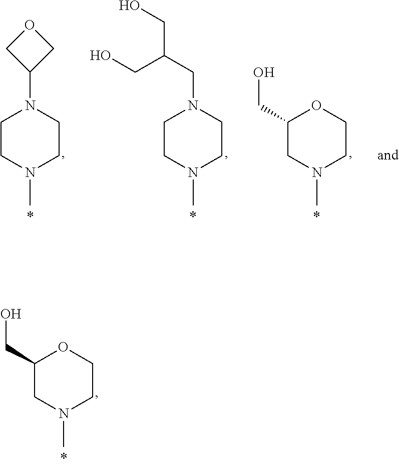
* indicates the carbon atom of the indicated phenyl ring to which R¹ is attached,
R² is H or 2-hydroxyethoxyl,
R³ is H or methyl, and
R⁴ is H or methyl.

4. The method of claim 3, wherein the Syk inhibitor is a compound of the formula: 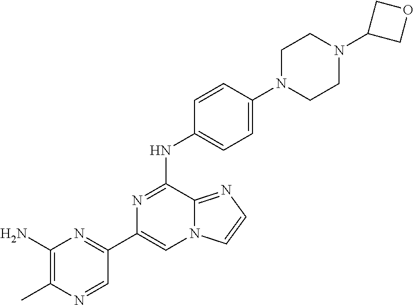 [SMILES: Cc1ncc(-c2cn3ccnc3c(Nc3ccc(N4CCN(C5COC5)CC4)cc3)n2)nc1N]
or a pharmaceutically acceptable salt or hydrate thereof, 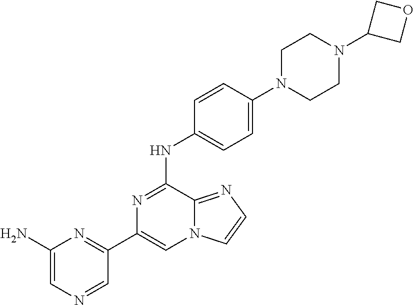 [SMILES: Nc1cncc(-c2cn3ccnc3c(Nc3ccc(N4CCN(C5COC5)CC4)cc3)n2)n1]
or a pharmaceutically acceptable salt or hydrate thereof, 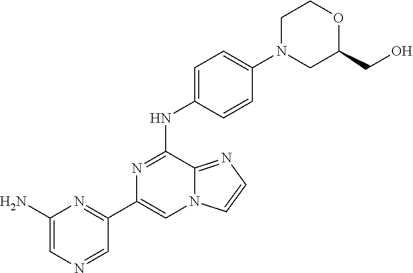 [SMILES: Nc1cncc(-c2cn3ccnc3c(Nc3ccc(N4CCO[C@@H](CO)C4)cc3)n2)n1]
or a pharmaceutically acceptable salt or hydrate thereof, 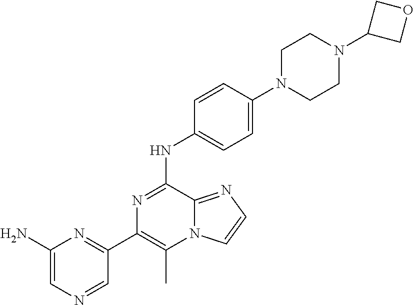 [SMILES: Cc1c(-c2cncc(N)n2)nc(Nc2ccc(N3CCN(C4COC4)CC3)cc2)c2nccn12]
or a pharmaceutically acceptable salt or hydrate thereof, 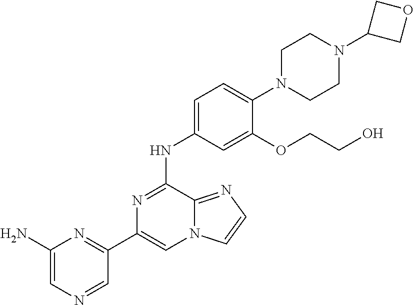 [SMILES: Nc1cncc(-c2cn3ccnc3c(Nc3ccc(N4CCN(C5COC5)CC4)c(OCCO)c3)n2)n1]
or a pharmaceutically acceptable salt or hydrate thereof, 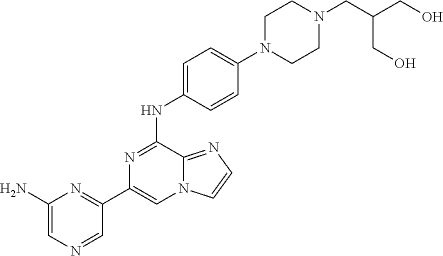 [SMILES: Nc1cncc(-c2cn3ccnc3c(Nc3ccc(N4CCN(CC(CO)CO)CC4)cc3)n2)n1]
or a pharmaceutically acceptable salt or hydrate thereof, or 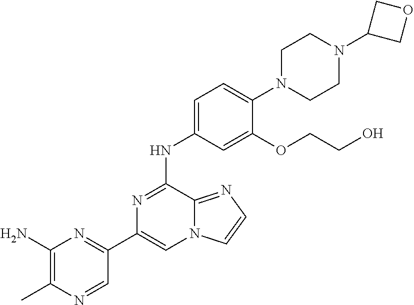 [SMILES: Cc1ncc(-c2cn3ccnc3c(Nc3ccc(N4CCN(C5COC5)CC4)c(OCCO)c3)n2)nc1N]
or a pharmaceutically acceptable salt or hydrate thereof.

5. The method of claim 1, wherein the additional therapeutic agents are idelalisib and obinutuzumab.

6. The method of claim 1, wherein the additional therapeutic agents are
a P13K inhibitor, or a pharmaceutically acceptable salt or hydrate thereof; and
obinutuzumab.

7. The method of claim 1, wherein the additional therapeutic agents are idelalisib and ABT-199.

8. The method of claim 1, wherein the additional therapeutic agents are obinutuzumab
and a Syk inhibitor of the formula: 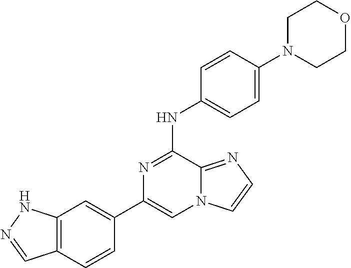 [SMILES: c1cn2cc(-c3ccc4cn[nH]c4c3)nc(Nc3ccc(N4CCOCC4)cc3)c2n1]
or a pharmaceutically effective salt or hydrate thereof, or of the formula: 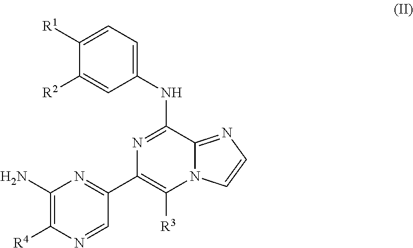
or a pharmaceutically acceptable salt or hydrate thereof, wherein
R¹ is selected from the group consisting of 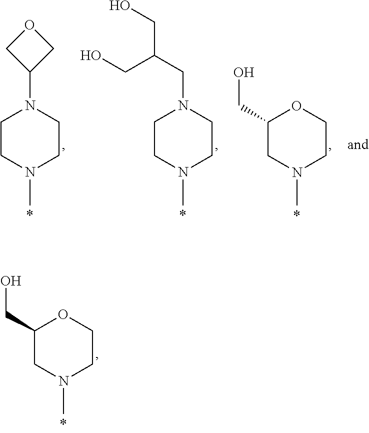
* indicates the carbon atom of the indicated phenyl ring of Formula II to which R¹ is attached,
R² is H or 2-hydroxyethoxyl,
R³ is H or methyl, and
R⁴ is H or methyl.

9. The method of claim 1, wherein the additional therapeutic agents are
ABT-199;
and a Syk inhibitor of the formula: 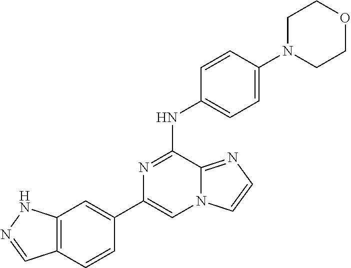 [SMILES: c1cn2cc(-c3ccc4cn[nH]c4c3)nc(Nc3ccc(N4CCOCC4)cc3)c2n1]
or a pharmaceutically acceptable salt or hydrate thereof, or of the formula: 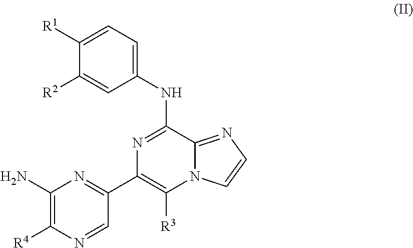
or a pharmaceutically acceptable salt or hydrate thereof, wherein
R¹ is selected from the group consisting of 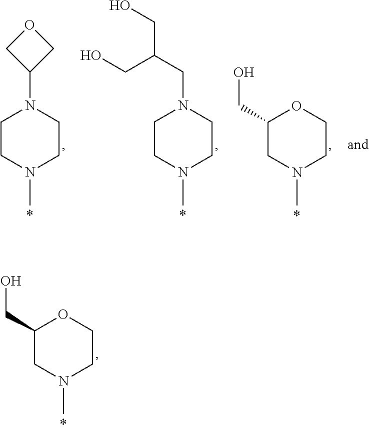
* indicates the carbon atom of the indicated phenyl ring of Formula II to which R¹ is attached,
R² is H or 2-hydroxyethoxyl,
R³ is H or methyl, and
R⁴ is H or methyl.

10. The method of claim 9, wherein the Syk inhibitor is a compound of the formula: 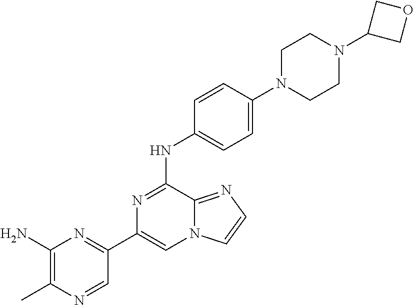 [SMILES: Cc1ncc(-c2cn3ccnc3c(Nc3ccc(N4CCN(C5COC5)CC4)cc3)n2)nc1N]
or a pharmaceutically acceptable salt or hydrate thereof, 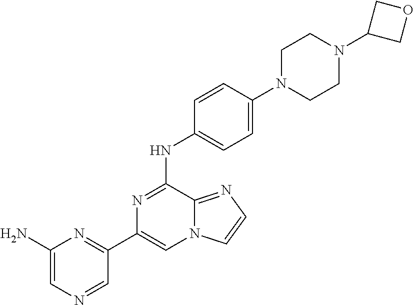 [SMILES: Nc1cncc(-c2cn3ccnc3c(Nc3ccc(N4CCN(C5COC5)CC4)cc3)n2)n1]
or a pharmaceutically acceptable salt or hydrate thereof, 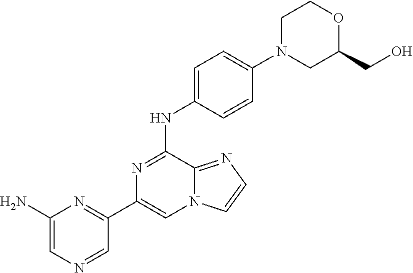 [SMILES: Nc1cncc(-c2cn3ccnc3c(Nc3ccc(N4CCO[C@@H](CO)C4)cc3)n2)n1]
or a pharmaceutically acceptable salt or hydrate thereof, 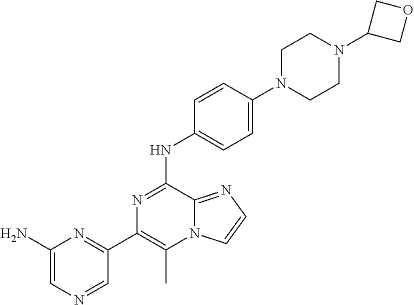 [SMILES: Cc1c(-c2cncc(N)n2)nc(Nc2ccc(N3CCN(C4COC4)CC3)cc2)c2nccn12]
or a pharmaceutically acceptable salt or hydrate thereof, 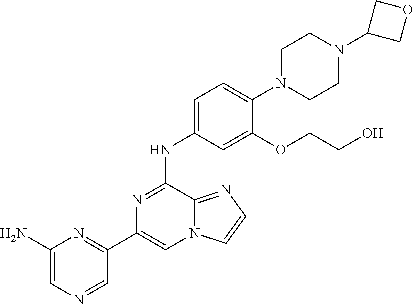 [SMILES: Nc1cncc(-c2cn3ccnc3c(Nc3ccc(N4CCN(C5COC5)CC4)c(OCCO)c3)n2)n1]
or a pharmaceutically acceptable salt or hydrate thereof, 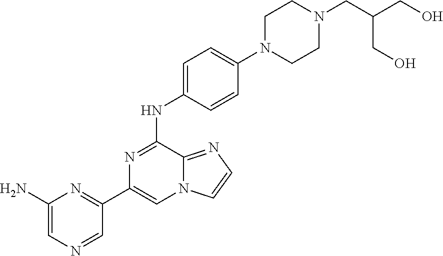 [SMILES: Nc1cncc(-c2cn3ccnc3c(Nc3ccc(N4CCN(CC(CO)CO)CC4)cc3)n2)n1]
or a pharmaceutically acceptable salt or hydrate thereof, or 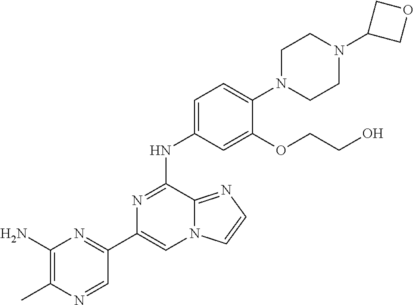 [SMILES: Cc1ncc(-c2cn3ccnc3c(Nc3ccc(N4CCN(C5COC5)CC4)c(OCCO)c3)n2)nc1N]
or a pharmaceutically acceptable salt or hydrate thereof.

11. A method for treating cancer in a human in need thereof, comprising administering to the human a combination of therapeutically effective amounts of ABT-199 and obinutuzumab, and a therapeutically effective amount of an Syk inhibitor of the formula: 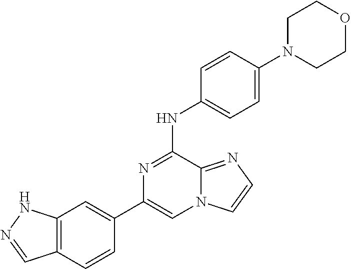 [SMILES: c1cn2cc(-c3ccc4cn[nH]c4c3)nc(Nc3ccc(N4CCOCC4)cc3)c2n1]
or a pharmaceutically effective salt or hydrate thereof, or of the formula: 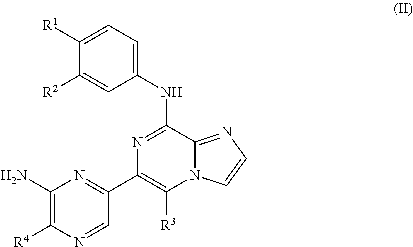
or a pharmaceutically effective salt or hydrate thereof, wherein
R¹ is selected from the group consisting of 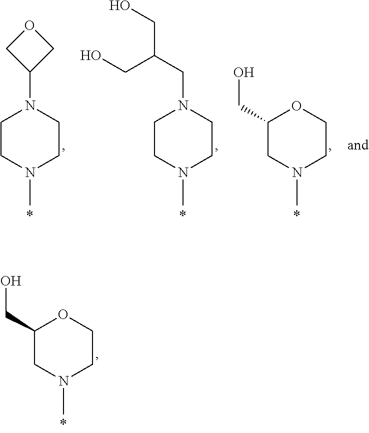
* indicates the carbon atom of the indicated phenyl ring of Formula II to which R¹ is attached,
R² is H or 2-hydroxyethoxyl,
R³ is H or methyl, and
R⁴ is H or methyl.

12. The method of claim 11, wherein the Syk inhibitor is a compound of the formula: 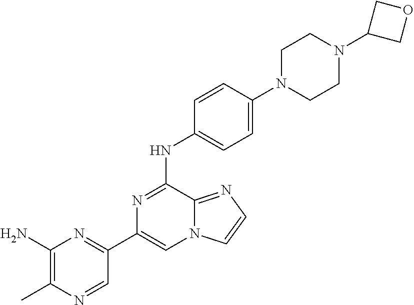 [SMILES: Cc1ncc(-c2cn3ccnc3c(Nc3ccc(N4CCN(C5COC5)CC4)cc3)n2)nc1N]
or a pharmaceutically acceptable salt or hydrate thereof, 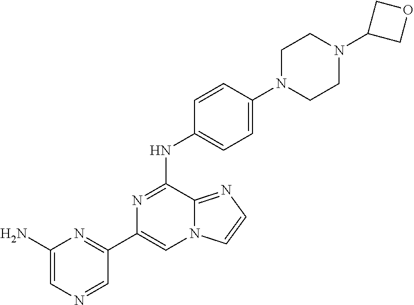 [SMILES: Nc1cncc(-c2cn3ccnc3c(Nc3ccc(N4CCN(C5COC5)CC4)cc3)n2)n1]
or a pharmaceutically acceptable salt or hydrate thereof, 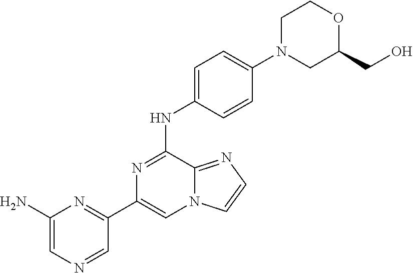 [SMILES: Nc1cncc(-c2cn3ccnc3c(Nc3ccc(N4CCO[C@@H](CO)C4)cc3)n2)n1]
or a pharmaceutically acceptable salt or hydrate thereof, 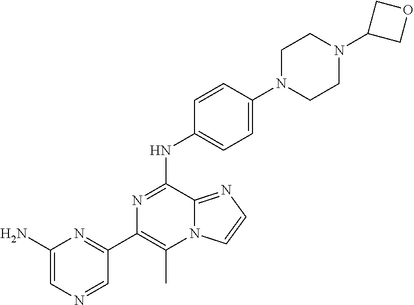 [SMILES: Cc1c(-c2cncc(N)n2)nc(Nc2ccc(N3CCN(C4COC4)CC3)cc2)c2nccn12]
or a pharmaceutically acceptable salt or hydrate thereof, 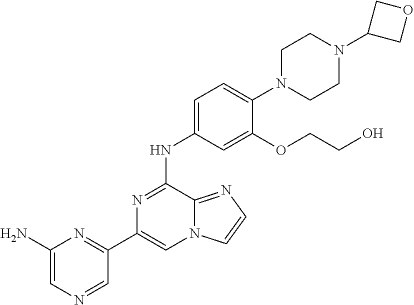 [SMILES: Nc1cncc(-c2cn3ccnc3c(Nc3ccc(N4CCN(C5COC5)CC4)c(OCCO)c3)n2)n1]
or a pharmaceutically acceptable salt or hydrate thereof, 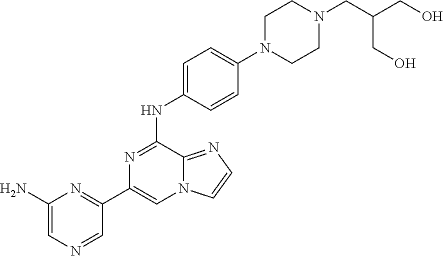 [SMILES: Nc1cncc(-c2cn3ccnc3c(Nc3ccc(N4CCN(CC(CO)CO)CC4)cc3)n2)n1]
or a pharmaceutically acceptable salt or hydrate thereof, or 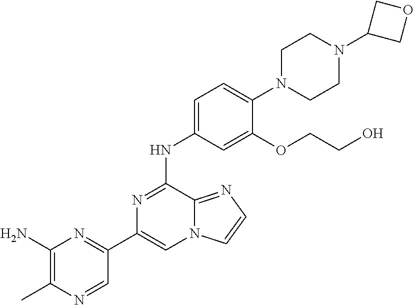 [SMILES: Cc1ncc(-c2cn3ccnc3c(Nc3ccc(N4CCN(C5COC5)CC4)c(OCCO)c3)n2)nc1N]
or a pharmaceutically acceptable salt or hydrate thereof.

13. The method of claim 1, wherein the additional therapeutic agents are obinutuzumab and ABT-199.

14. A method for treating cancer in a human in need thereof, comprising administering to the human a combination of therapeutically effective amounts of idelalisib and obinutuzumab, and a therapeutically effective amount of ABT-199.

15. The method of claim 1, wherein the additional therapeutic agent is idelalisib.
